(19) United States
(12) Patent Application Publication
(10) Pub. No.: US 2026/0194813 A1
(43) Pub. Date: Jul. 9, 2026

(54) ACTINIC RAY-SENSITIVE OR RADIATION-SENSITIVE RESIN COMPOSITION, ACTINIC RAY-SENSITIVE OR RADIATION-SENSITIVE FILM, PATTERN FORMING METHOD, AND METHOD FOR PRODUCING ELECTRONIC DEVICE

(71) Applicant: FUJIFILM Corporation, Tokyo (JP)

(72) Inventors: Tsutomu Yoshimura, Shizuoka (JP); Naoya Hatakeyama, Shizuoka (JP); Masafumi Kojima, Shizuoka (JP); Akiyoshi Goto, Shizuoka (JP); Takahiro Mori, Shizuoka (JP); Nobuhiro Hiura, Shizuoka (JP); Kazuhiro Marumo, Shizuoka (JP)

(21) Appl. No.: 19/557,442

(22) Filed: Mar. 5, 2026

Related U.S. Application Data
(63) Continuation of application No. PCT/JP2024/030302, filed on Aug. 26, 2024.

(30) Foreign Application Priority Data
Sep. 6, 2023 (JP) 2023-144804
Nov. 8, 2023 (JP) 2023-190956
Dec. 22, 2023 (JP) 2023-217276
Apr. 26, 2024 (JP) 2024-072090

(51) Int. Cl. G03F 7/004; C07C 25/18; C07C 309/17; C07C 309/42; C07C 309/65; C07C 311/48; C07C 311/51; C07C 381/12; C07D 307/12; C07D 309/12; C07D 327/06; C07D 327/08; C07D 333/46; C07D 333/76; C07D 493/18; C08F 212/14; C08F 220/18; C08F 220/22; C08F 220/28; C08F 220/38; C08F 222/14; C08F 226/06; C08F 232/08; G03F 7/00; G03F 7/039; G03F 7/20; H10P 76/20

(52) U.S. Cl. CPC G03F 7/0045; C07C 25/18; C07C 309/17; C07C 309/42; C07C 309/65; C07C 311/48; C07C 311/51; C07C 381/12; C07D 307/12; C07D 309/12; C07D 327/06; C07D 327/08; C07D 333/46; C07D 333/76; C07D 493/18; C08F 212/22; C08F 212/24; C08F 220/1806; C08F 220/1807; C08F 220/1808; C08F 220/1809; C08F 220/1811; C08F 220/1818; C08F 220/22; C08F 220/281; C08F 220/282; C08F 220/283; C08F 220/382; C08F 222/145; C08F 226/06; C08F 232/08; G03F 7/0046; G03F 7/0397; C07C 2601/08; C07C 2601/14; C07C 2603/74; C08F 2800/10; G03F 7/2004; G03F 7/70033; H10P 76/2041

(57) ABSTRACT

The present invention provides an actinic ray-sensitive or radiation-sensitive resin composition including an acid-decomposable resin (A) and the following compound (B), an actinic ray-sensitive or radiation-sensitive resin film formed using the composition, and a pattern forming method and a method for producing an electronic device that use the composition, Compound (B): a compound that has structural moieties X and Y described in the specification and that generates an acid including a first acidic moiety derived from the structural moiety X and a second acidic moiety derived from the structural moiety Y upon irradiation with actinic rays or radiation, in which a compound PI obtained by replacing cationic moieties with H⁺ has acid dissociation constants a1 and a2 derived from the acidic moieties, a2 is larger than al, and an anionic moiety in the structural moiety Y is represented by a formula (1) described in the specification.

### CROSS REFERENCE TO RELATED APPLICATION

This is a continuation of International Application No. PCT/JP2024/030302 filed on Aug. 26, 2024, and claims priorities from Japanese Patent Application No. 2023-144804 filed on Sep. 6, 2023, Japanese Patent Application No. 2023-190956 filed on Nov. 8, 2023, Japanese Patent Application No. 2023-217276 filed on Dec. 22, 2023 and Japanese Patent Application No. 2024-072090 filed on Apr. 26, 2024, the entire disclosures of which are incorporated herein by reference.

### BACKGROUND OF THE INVENTION

### 1. Field of the Invention

The present invention relates to an actinic ray-sensitive or radiation-sensitive resin composition, an actinic ray-sensitive or radiation-sensitive film, a pattern forming method, and a method for producing an electronic device.

### 2. Description of the Related Art

In a manufacturing process of semiconductor devices such as ICs (Integrated Circuits) and LSIs (Large-Scale Integrated Circuits), microfabrication by lithography using a photosensitive composition is performed.

An example of the lithography method is a method including forming a resist film using a photosensitive composition, subsequently exposing the obtained film, and subsequently developing the film. In particular, in recent years, studies have been conducted on using EB (Electron Beam) or EUV (Extreme ultraviolet) in addition to ArF excimer laser during exposure, and actinic ray-sensitive or radiation-sensitive resin compositions suitable for EUV exposure have been developed.

In the formation of a resist pattern using EUV (having a wavelength of 13.5 nm) or an electron beam for the purpose of forming a fine pattern, requirements for various performances are stricter than those in the case of using existing ArF light (having a wavelength of 193 nm) or the like.

A resist composition used for forming a resist pattern using EUV, an electron beam, or the like often includes a photoacid generator and a photodegradable base. However, in a resist film formed using the resist composition, since the distribution of the photoacid generator and the photodegradable base varies, the concentration of an acid generated by exposure becomes non-uniform, and the solubility of the resist film in a developer varies. As a result, the line width of the obtained resist pattern varies, and the roughness performance deteriorates.

As a method for suppressing such a roughness performance deterioration caused by the variation in the material distribution of the photoacid generator and the photodegradable base, for example, JP2019-014704A describes a resist composition including a salt having a specific structure in which a photoacid generator and a photodegradable base are linked.

### SUMMARY OF THE INVENTION

The salt described in JP2019-014704A exhibits excellent roughness performance because a portion corresponding to the photodegradable base is in a carboxylic acid anion form, and thus the salt is hydrophilic and resistant to swelling in developers, and because its high basicity and high acid-trapping ability result in high deprotection contrast of a protective group in an acid-decomposable group of a resin. However, studies conducted by the inventors of the present invention have revealed that, due to the hydrophilic and highly basic properties of the salt, the solubility of the salt alone or the matrix of the salt and the resin in developers decreases, resulting in defects in the pattern.

An object of the present invention is to provide an actinic ray-sensitive or radiation-sensitive resin composition that can suppress the occurrence of defects and exhibits excellent roughness performance in the formation of an ultrafine pattern (for example, a line-and-space pattern having a line width of 25 nm or less or a hole pattern having a hole diameter of 25 nm or less), an actinic ray-sensitive or radiation-sensitive resin film formed using the actinic ray-sensitive or radiation-sensitive resin composition, and a pattern forming method and a method for producing an electronic device that use the actinic ray-sensitive or radiation-sensitive resin composition.

The inventors of the present invention have found that the above object can be achieved by the following configurations.

[1]

An actinic ray-sensitive or radiation-sensitive resin composition including:
an acid-decomposable resin (A); and
the following compound (B) which generates an acid upon irradiation with actinic rays or radiation.

### Compound (B):

A compound which has the following structural moiety X and the following structural moiety Y and which generates an acid including the following first acidic moiety derived from the structural moiety X and the following second acidic moiety derived from the structural moiety Y upon irradiation with actinic rays or radiation.

Structural moiety X: A structural moiety which is constituted by an anionic moiety A₁⁻ and a cationic moiety M₁⁺ and which forms the first acidic moiety represented by HA₁ upon irradiation with actinic rays or radiation

Structural moiety Y: A structural moiety which is constituted by an anionic moiety A₂⁻ and a cationic moiety M₂⁺ and which forms the second acidic moiety represented by HA₂ upon irradiation with actinic rays or radiation

The compound (B) satisfies conditions I and II below.

Condition I: A compound PI obtained by replacing the cationic moiety M₁⁺ in the structural moiety X and the cationic moiety M₂⁺ in the structural moiety Y in the compound (B) with H⁺ has an acid dissociation constant a1 (pKa) derived from an acidic moiety represented by HA₁ obtained by replacing the cationic moiety M₁⁺ in the structural moiety X with H⁺ and an acid dissociation constant a2 (pKa) derived from an acidic moiety represented by HA₂ obtained by replacing the cationic moiety M₂⁺ in the structural moiety Y with H⁺, and the acid dissociation constant a2 is larger than the acid dissociation constant a1.

Condition II: In the compound (B), the anionic moiety A₂⁻ in the structural moiety Y is an anionic moiety including a partial structure represented by a formula (1) below.

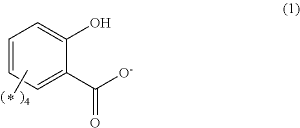

In the formula (1), * represents a bonding site to another atom.

[2]

The actinic ray-sensitive or radiation-sensitive resin composition according to [1], wherein the acid dissociation constant a1 (pKa) is −1.00 or less.

[3]

The actinic ray-sensitive or radiation-sensitive resin composition according to [1] or [2], wherein in the compound (B), the anionic moiety A₁⁻ in the structural moiety X is an anionic moiety including a partial structure represented by any one of formulae (2) to (5) below.

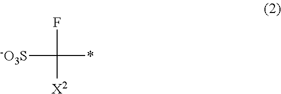

In the formula (2), X² represents a hydrogen atom, a halogen atom, or an organic group. * represents a bonding site to another atom.

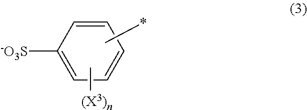

In the formula (3), X³ represents an electron-withdrawing group. n represents an integer of 1 to 4. When n is an integer of 2 or more, a plurality of X³'s may be the same or different. * represents a bonding site to another atom.

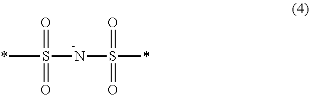 [SMILES: *S(=O)(=O)NS(*)(=O)=O]

In the formula (4), * represents a bonding site to another atom.

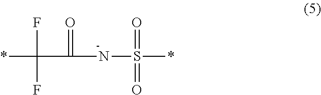 [SMILES: *C(F)(F)C(=O)NS(*)(=O)=O]

In the formula (5), * represents a bonding site to another atom.

[4]

The actinic ray-sensitive or radiation-sensitive resin composition according to any one of [1] to [3], wherein the compound (B) has a halogen atom in a cationic moiety.

[5]

The actinic ray-sensitive or radiation-sensitive resin composition according to any one of [1] to [4], wherein the acid-decomposable resin (A) includes a repeating unit having a phenolic hydroxy group.

[6]

An actinic ray-sensitive or radiation-sensitive film formed using the actinic ray-sensitive or radiation-sensitive resin composition according to any one of [1] to [5].

[7]

A pattern forming method having:
forming an actinic ray-sensitive or radiation-sensitive film on a substrate using the actinic ray-sensitive or radiation-sensitive resin composition according to any one of [1] to [5];
exposing the actinic ray-sensitive or radiation-sensitive film; and
developing the exposed actinic ray-sensitive or radiation-sensitive film using a developer to form a pattern.
   [8]

A method for producing an electronic device, the method including the pattern forming method according to [7].

According to the present invention, it is possible to provide an actinic ray-sensitive or radiation-sensitive resin composition that can suppress the occurrence of defects and exhibits excellent roughness performance in the formation of an ultrafine pattern (for example, a line-and-space pattern having a line width of 25 nm or less or a hole pattern having a hole diameter of 25 nm or less), an actinic ray-sensitive or radiation-sensitive resin film formed using the actinic ray-sensitive or radiation-sensitive resin composition, and a pattern forming method and a method for producing an electronic device that use the actinic ray-sensitive or radiation-sensitive resin composition.

### DESCRIPTION OF THE PREFERRED EMBODIMENTS

The present invention will be described in detail below.

Constituent features may be described below on the basis of representative embodiments of the present invention; however, the present invention is not limited to such embodiments.

Regarding notations of groups (atomic groups) in the present specification, a notation without specifying whether substituted or unsubstituted also encompasses a group including a substituent as well as a group having no substituent without departing from the spirit and scope of the present invention. For example, an “alkyl group” encompasses not only an alkyl group having no substituent (an unsubstituted alkyl group) but also an alkyl group having a substituent (a substituted alkyl group). In the present specification, an “organic group” refers to a group including at least one carbon atom.

As the substituent, a monovalent substituent is preferable unless otherwise specified.

In the present specification, in the case of using a phrase “may have a substituent”, the type of the substituent, the position of the substituent, and the number of substituents are not particularly limited. The number of the substituents may be, for example, one, two, three, or more. Examples of the substituent include monovalent non-metallic atomic groups excluding a hydrogen atom, and, for example, the substituent can be selected from the group consisting of the following substituents T.

### (Substituents T)

Examples of the substituents T include halogen atoms such as a fluorine atom, a chlorine atom, a bromine atom, and an iodine atom; alkoxy groups such as a methoxy group, an ethoxy group, and a tert-butoxy group; aryloxy groups such as a phenoxy group and a p-tolyloxy group; alkoxycarbonyl groups such as a methoxycarbonyl group and a butoxycarbonyl group; aryloxycarbonyl groups such as a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; alkylsulfanyl groups such as a methylsulfanyl group and a tert-butylsulfanyl group; arylsulfanyl groups such as a phenylsulfanyl group and a p-tolylsulfanyl group; alkyl groups; cycloalkyl groups; aryl groups; heteroaryl groups; a hydroxy group; a carboxy group; a formyl group; a sulfo group; a cyano group; alkylaminocarbonyl groups; arylaminocarbonyl groups; a sulfonamide group; silyl groups; an amino group; monoalkylamino groups; dialkylamino groups; arylamino groups; a nitro group; and combinations thereof.

In the present specification, “actinic rays” or “radiation” means, for example, a bright line spectrum of a mercury lamp, far ultraviolet rays typified by excimer laser, extreme ultraviolet rays (EUV: Extreme Ultraviolet), X-rays, and an electron beam (EB: Electron Beam).

In the present specification, “light” means actinic rays or radiation.

In the present specification, unless otherwise specified, the “exposure” includes not only exposure to a bright line spectrum of a mercury lamp, far ultraviolet rays typified by excimer laser, extreme ultraviolet rays, X-rays, or the like, but also patterning using an electron beam or a particle beam such as an ion beam.

In the present specification, a range of numerical values expressed with “to” means a range that includes a numerical value before “to” as a lower limit value and a numerical value after “to” as an upper limit value.

The bonding direction of a divalent linking group expressed in the present specification is not limited unless otherwise specified. For example, in a compound represented by a formula “X—Y—Z” where Y is —COO—, Y may be —CO—O— or —O—CO—. The compound may be “X—CO—O—Z” or “X—O—CO—Z”.

In the present specification, (meth)acrylate represents acrylate and methacrylate, and (meth)acryl represents acryl and methacryl.

In the present specification, the weight-average molecular weight (Mw), the number-average molecular weight (Mn), and the molecular weight dispersity (also referred to as “molecular weight distribution”) (Mw/Mn) are defined as values in terms of polystyrene determined, using a GPC (Gel Permeation Chromatography) apparatus (HLC-8120GPC manufactured by Tosoh Corporation), by GPC measurement (solvent: tetrahydrofuran, amount of flow (amount of sample injected): 10 μL, column: TSK gel Multipore HXL-M manufactured by Tosoh Corporation, column temperature: 40° C., flow rate: 1.0 mL/min, detector: differential refractive index detector (Refractive Index Detector)).

In the present specification, the acid dissociation constant (pKa) represents pKa in an aqueous solution, and specifically, is a value determined by calculation using the following software package 1 on the basis of the Hammett substituent constant and a database of values in publicly known documents.

Software package 1: Advanced Chemistry Development (ACD/Labs) Software V8.14 for Solaris (1994-2007 ACD/Labs)

The pKa can also be determined by molecular orbital calculation methods. A specific method thereof may be a method of calculating the pKa by calculating H⁺ dissociation free energy in an aqueous solution on the basis of a thermodynamic cycle. With regard to the method of calculating the H⁺ dissociation free energy, the calculation can be performed by, for example, DFT (density functional theory); however, various other methods have been reported in documents etc., and the method is not limited to this. There are a plurality of pieces of software capable of performing DFT, and an example thereof is Gaussian 16.

In the present specification, as described above, the pKa refers to a value determined by calculation using the software package 1 on the basis of the Hammett substituent constant and the database of values in publicly known documents; however, when the pKa cannot be calculated by this method, a value determined using Gaussian16 based on DFT (density functional theory) is employed.

In the present specification, the pKa refers to “pKa in an aqueous solution” as described above; however when the pKa in an aqueous solution cannot be calculated, “pKa in a dimethyl sulfoxide (DMSO) solution” is employed.

In the present specification, the “solid contents” mean components that form an actinic ray-sensitive or radiation-sensitive film (typically, a resist film), and do not include a solvent. In addition, even if a component that forms an actinic ray-sensitive or radiation-sensitive film is in a liquid state, the component is considered as a solid content.

### [Actinic Ray-Sensitive or Radiation-Sensitive Resin Composition]

Hereinafter, an actinic ray-sensitive or radiation-sensitive resin composition of the present invention will be described.

The actinic ray-sensitive or radiation-sensitive resin composition of the present invention (hereinafter, also referred to as the “composition of the present invention”) includes an acid-decomposable resin (A) and the following compound (B) that generates an acid upon irradiation with actinic rays or radiation.

### Compound (B):

A compound that has the following structural moiety X and the following structural moiety Y and that generates an acid including the following first acidic moiety derived from the structural moiety X and the following second acidic moiety derived from the structural moiety Y upon irradiation with actinic rays or radiation.

Structural moiety X: A structural moiety that is constituted by an anionic moiety A₁⁻ and a cationic moiety M₁⁺ and that forms the first acidic moiety represented by HA₁ upon irradiation with actinic rays or radiation

Structural moiety Y: A structural moiety that is constituted by an anionic moiety A₂⁻ and a cationic moiety M₂⁺ and that forms the second acidic moiety represented by HA₂ upon irradiation with actinic rays or radiation

The compound (B) satisfies conditions I and II below.

Condition I: A compound PI obtained by replacing the cationic moiety M₁⁺ in the structural moiety X and the cationic moiety M₂⁺ in the structural moiety Y in the compound (B) with H⁺ has an acid dissociation constant a1 derived from an acidic moiety represented by HA₁ obtained by replacing the cationic moiety M₁⁺ in the structural moiety X with H⁺ and an acid dissociation constant a2 derived from an acidic moiety represented by HA₂ obtained by replacing the cationic moiety M₂⁺ in the structural moiety Y with H⁺, and the acid dissociation constant a2 is larger than the acid dissociation constant a1.

Condition II: In the compound (B), the anionic moiety A₂⁻ in the structural moiety Y is an anionic moiety including a partial structure represented by a formula (1) below.

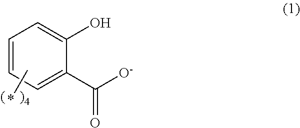

In the formula (1), * represents a bonding site to another atom.

The reason why the composition of the present invention can suppress the occurrence of defects and exhibits excellent roughness performance in the formation of an ultrafine pattern (for example, a line-and-space pattern having a line width of 25 nm or less or a hole pattern having a hole diameter of 25 nm or less) is not necessarily clear, but the inventors of the present invention presume as follows.

The compound (B) included in the composition of the present invention is a compound having both functions of a photoacid generator and a photodegradable base, and a portion corresponding to the photodegradable base is in a salicylate anion form. With the salicylate anion form, the compound (B) has moderate hydrophilicity and moderate basicity. Therefore, it is considered that the roughness performance can be improved while the defects that occur with a carboxylic acid anion form, such as the salt described in JP2019-014704A, is suppressed.

The composition of the present invention is typically a resist composition, and may be either a positive resist composition or a negative resist composition. The composition of the present invention may be a resist composition for alkali development or a resist composition for organic solvent development.

The composition of the present invention may be a chemical amplification resist composition or a non-chemical amplification resist composition. The composition of the present invention is typically a chemical amplification resist composition.

The composition of the present invention can be used to form an actinic ray-sensitive or radiation-sensitive film. The actinic ray-sensitive or radiation-sensitive film formed using the composition of the present invention is typically a resist film.

First, various components of the composition of the present invention will be described in detail below.

### [Compound (B)]

The compound (B) (hereinafter, also referred to as “photoacid generator (B)” included in the composition of the present invention will be described.

The compound (B) is a compound that has the above structural moiety X and the above structural moiety Y and that generates an acid including the above first acidic moiety derived from the structural moiety X and the above second acidic moiety derived from the structural moiety Y upon irradiation with actinic rays or radiation. However, the compound (B) satisfies the conditions I and II described above.

The compound (B) is a compound having the structural moiety X and the structural moiety Y. The compound (B) may have one or more structural moieties X and one or more structural moieties Y, and is preferably a compound having one structural moiety X and one structural moiety Y, or a compound having two structural moieties X and one structural moiety Y.

The condition I will be more specifically described below.

When the compound (B) is, for example, a compound that generates an acid having one first acidic moiety derived from the structural moiety X and one second acidic moiety derived from the structural moiety Y, the compound PI corresponds to a “compound having HA₁ and HA₂”.

The acid dissociation constant a1 and the acid dissociation constant a2 of the compound PI are more specifically described as follows. In determination of the acid dissociation constants of the compound PI, the pKa determined when the compound PI turns into a “compound having A₁⁻ and HA₂” is the acid dissociation constant a1, and the pKa determined when the “compound having A₁⁻ and HA₂” turns into a “compound having A₁⁻ and A₂⁻” is the acid dissociation constant a2.

When the compound (B) is, for example, a compound that generates an acid having two first acidic moieties derived from the structural moiety X and one second acidic moiety derived from the structural moiety Y, the compound PI corresponds to a “compound having two HA₁'s and one HA₂”.

In determination of the acid dissociation constants of the compound PI, the acid dissociation constant determined when the compound PI turns into a “compound having one A₁⁻, one HA₁, and one HA₂” and the acid dissociation constant determined when the “compound having one A₁⁻, one HA₁, and one HA₂” turns into a “compound having two A₁⁻ 's and one HA₂” each correspond to the above-described acid dissociation constant a1. The acid dissociation constant determined when the “compound having two A₁⁻ 's and one HA₂” turns into a “compound having two A₁⁻ 's and A₂⁻” corresponds to the acid dissociation constant a2. That is, when the compound PI has a plurality of acid dissociation constants derived from acidic moieties represented by HA₁ obtained by replacing the cationic moiety M₁⁺ in the structural moiety X with H⁺, the value of the acid dissociation constant a2 is larger than the largest value among the plurality of acid dissociation constants a1. Note that, when the acid dissociation constant determined when the compound PI turns into a “compound having one A₁⁻, one HA₁, and one HA₂” is defined as aa and the acid dissociation constant determined when the “compound having one A₁⁻, one HA₁, and one HA₂” turns into a “compound having two A₁⁻ 's and one HA₂” is defined as ab, the relation between aa and ab satisfies aa<ab.

The acid dissociation constant a1 and the acid dissociation constant a2 can be determined by the above-described method of measuring an acid dissociation constant.

The compound PI corresponds to an acid generated when the compound (B) is irradiated with actinic rays or radiation.

When the compound (B) has two or more structural moieties X, the structural moieties X may be the same or different. Two or more A₁⁻ 's and two or more M₁⁺'s may be individually the same or different.

In the compound (B), A₁⁻ and A₂⁻, and M₁⁺ and M₂⁺ may be individually the same or different, but A₁⁻ and A₂⁻ are preferably different.

In the compound PI, the difference (absolute value) between the acid dissociation constant a1 (in the case where a plurality of acid dissociation constants a1 are present, the maximum value thereof) and the acid dissociation constant a2 is preferably 0.10 or more, more preferably 0.50 or more, still more preferably 1.00 or more. The upper limit value of the difference (absolute value) between the acid dissociation constant a1 (in the case where a plurality of acid dissociation constants a1 are present, the maximum value thereof) and the acid dissociation constant a2 is not particularly limited, but is, for example, 16 or less.

In the compound PI, the acid dissociation constant a2 is an acid dissociation constant derived from an acidic moiety constituted by an anionic moiety including a partial structure represented by a formula (1) described later and H⁺, and is preferably 20 or less, more preferably 15 or less. The lower limit value of the acid dissociation constant a2 is preferably −4.00 or more.

In the compound PI, the acid dissociation constant a1 is preferably 2.00 or less, more preferably 0 or less, still more preferably −1.00 or less. The lower limit value of the acid dissociation constant a1 is preferably −20.00 or more.

The anionic moiety A₂⁻ will be described.

In the compound (B), the anionic moiety A₂⁻ in the structural moiety Y is an anionic moiety including a partial structure represented by the following formula (1), as described in the condition II.

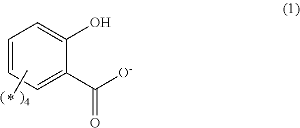

In the formula (1), * represents a bonding site to another atom.

In a preferred embodiment, the anionic moiety including the partial structure represented by the formula (1) is preferably an anionic functional group represented by a formula (1a) described later.

Next, the anionic moiety A₁⁻ will be described.

The anionic moiety A₁⁻ is a structural moiety including a negatively charged atom or atomic group, is preferably capable of forming an acidic moiety having a smaller acid dissociation constant than that of the anionic moiety A₂⁻, and may be, for example, a structural moiety including a partial structure selected from the group consisting of formulae (CC-1) to (CC-7) below.

In the following formulae (CC-1) to (CC-7), * represents a bonding site.

In the formula (CC-2), each R^{A} represents a monovalent organic group. The monovalent organic group represented by R^{A} is not particularly limited and may be, for example, a cyano group, a trifluoromethyl group, or a methanesulfonyl group.

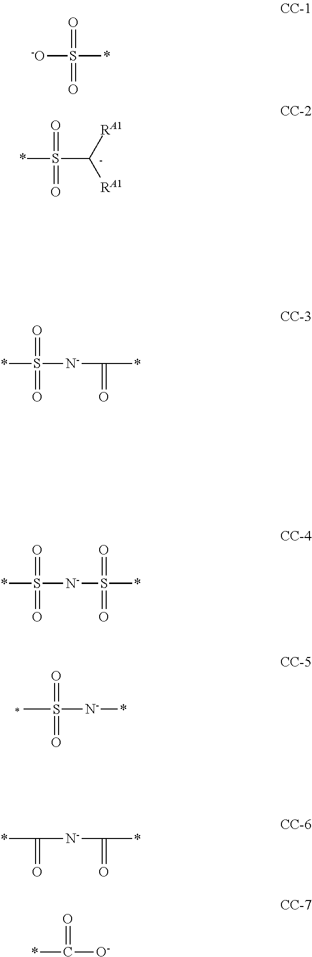

The anionic moiety A₁⁻ is preferably an anionic moiety including a partial structure represented by any one of formulae (2) to (5) below.

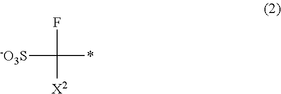

In the formula (2), X² represents a hydrogen atom, a halogen atom, or an organic group. * represents a bonding site to another atom.

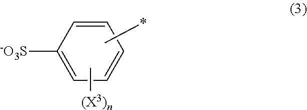

In the formula (3), X³ represents an electron-withdrawing group. n represents an integer of 1 to 4. When n is an integer of 2 or more, a plurality of X³'s present may be the same or different. * represents a bonding site to another atom.

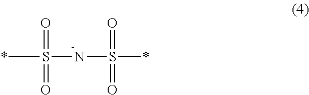 [SMILES: *S(=O)(=O)NS(*)(=O)=O]

In the formula (4), * represents a bonding site to another atom.

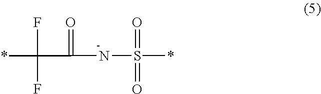 [SMILES: *C(F)(F)C(=O)NS(*)(=O)=O]

In the formula (5), * represents a bonding site to another atom.

In the formula (2), X² represents a hydrogen atom, a halogen atom, or an organic group.

The halogen atom represented by X² may be a fluorine atom, a chlorine atom, a bromine atom, an iodine atom, or the like and is preferably a fluorine atom.

The organic group represented by X² may be an alkyl group that has 1 to 15 carbon atoms and that may have a fluorine atom, preferably a perfluoroalkyl group having 1 to 10 carbon atoms, more preferably a perfluoroalkyl group having 1 to 6 carbon atoms.

X² preferably represents a halogen atom and more preferably represents a fluorine atom.

In the formula (3), X³ represents an electron-withdrawing group.

The electron-withdrawing group represented by X³ may be a group selected from the group consisting of a halogen atom (preferably a fluorine atom, a chlorine atom, a bromine atom, or an iodine atom), a halogenated hydrocarbon group, an alkoxycarbonyl group, an acyl group, an acyloxy group, a cyano group, a nitro group, and a heterocyclic group, or an aryl group substituted with a group selected from the above group.

X³ preferably has a Hammett rule substituent constant σp value of 0.05 or more, and is more preferably a halogen atom, a halogenated hydrocarbon group, a cyano group, or a nitro group, still more preferably a fluorine atom, a fluorinated hydrocarbon group, a cyano group, or a nitro group, still further more preferably a fluorine atom or a fluorinated hydrocarbon group (preferably a fluoroalkyl group having 1 to 10 carbon atoms), particularly preferably a fluorine atom.

Here, the Hammett rule substituent constant σ is a numerical value representing the effect of a substituent on the acid dissociation equilibrium constant of a substituted benzoic acid, and is a parameter indicating the strength of the electron-withdrawing property and electron-donating property of the substituent. The Hammett substituent constant σp value in the present specification means a substituent constant σ in the case where the substituent is located at the para position of benzoic acid.

As the Hammett substituent constant 6p value for each group in the present specification, the value described in the literature “Hansch et al., Chemical Reviews, 1991, Vol. 91, No. 2, 165-195” is employed. Note that, for a group whose Hammett substituent constant 6p value is not described in the above literature, the Hammett substituent constant 6p value can be calculated using the software “ACD/ChemSketch (ACD/Labs 8.00 Release Product Version: 8.08)” on the basis of the difference between the pKa of benzoic acid and the pKa of a benzoic acid derivative having the substituent at the para position.

In the formula (3), n represents an integer of 1 to 4, and is preferably 3 or 4.

The cationic moiety M₁⁺ and the cationic moiety M₂⁺ are structural moieties including a positively charged atom or atomic group and are, for example, singly charged organic cations.

The compound (B) preferably has a halogen atom in a cationic moiety, and M₁⁺ and M₂⁺ each preferably have a halogen atom.

The halogen atom included in the cationic moiety may be a fluorine atom, a chlorine atom, a bromine atom, an iodine atom, or the like and is preferably a fluorine atom or an iodine atom.

When the cationic moiety has a halogen atom, the absorption efficiency of EUV light or an electron beam and the solubility of the compound (B) in a developer can be enhanced, resulting in improved roughness performance and defect suppression performance.

M₁⁺ and M₂⁺ are each preferably a sulfonium cation or an iodonium cation.

The cations represented by M₁⁺ and M₂⁺ are not particularly limited. The valence of each of the cations may be mono-, di-, or higher valent. As the cation, a cation represented by a formula (ZaI) (hereinafter, also referred to as a “cation (ZaI)”) or a cation represented by a formula (ZaII) (hereinafter, also referred to as a “cation (ZaII)”) is preferable.

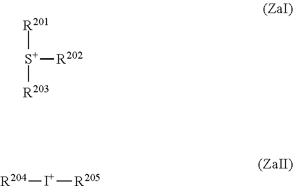

In the formula (ZaI), R²⁰¹, R²⁰², and R²⁰³ each independently represent an organic group.

The number of carbon atoms of each of the organic groups serving as R²⁰¹, R²⁰², and R²⁰³ is preferably 1 to 30, more preferably 1 to 20. Two of R²⁰¹ to R²⁰³ may be bonded together to form a ring structure, and the ring may include an oxygen atom, a sulfur atom, an ester group, an amide group, or a carbonyl group. Examples of the group formed by bonding two of R²⁰¹ to R²⁰³ together include alkylene groups (such as a butylene group and a pentylene group) and —CH₂—CH₂—O—CH₂—CH₂—.

Preferred embodiments of the organic cation in the formula (ZaI) include a cation (ZaI-1), a cation (ZaI-2), a cation (ZaI-3b), and a cation (ZaI-4b) described below.

First, the cation (ZaI-1) will be described.

The cation (ZaI-1) is an arylsulfonium cation in which at least one of R²⁰¹ to R²⁰³ in the formula (ZaI) is an aryl group.

In the arylsulfonium cation, all of R²⁰¹ to R²⁰³ may be aryl groups, or some of R²⁰¹ to R²⁰³ may be an aryl group and the remainder may be an alkyl group or a cycloalkyl group.

One of R²⁰¹ to R²⁰³ may be an aryl group and the other two of R²⁰¹ to R²⁰³ may be bonded together to form a ring structure, and the ring may include an oxygen atom, a sulfur atom, an ester group, an amide group, or a carbonyl group. Examples of the group formed by bonding two of R²⁰¹ to R²⁰³ together include alkylene groups in which one or more methylene groups may be substituted with an oxygen atom, a sulfur atom, an ester group, an amide group, and/or a carbonyl group (such as a butylene group, a pentylene group, and —CH₂—CH₂—O—CH₂—CH₂—).

Examples of the arylsulfonium cation include triarylsulfonium cations, diarylalkylsulfonium cations, aryldialkylsulfonium cations, diarylcycloalkylsulfonium cations, and aryldicycloalkylsulfonium cations.

The aryl group included in the arylsulfonium cation is preferably a phenyl group or a naphthyl group, more preferably a phenyl group. The aryl group may be an aryl group having a heterocyclic structure having, for example, an oxygen atom, a nitrogen atom, or a sulfur atom. Examples of the heterocyclic structure include a pyrrole residue, a furan residue, a thiophene residue, an indole residue, a benzofuran residue, and a benzothiophene residue. When the arylsulfonium cation has two or more aryl groups, the two or more aryl groups may be the same or different.

The alkyl group or the cycloalkyl group that the arylsulfonium cation optionally has is preferably a linear alkyl group having 1 to 15 carbon atoms, a branched alkyl group having 3 to 15 carbon atoms, or a cycloalkyl group having 3 to 15 carbon atoms, more preferably a methyl group, an ethyl group, a propyl group, a n-butyl group, a sec-butyl group, a t-butyl group, a cyclopropyl group, a cyclobutyl group, or a cyclohexyl group.

Substituents that the aryl group, the alkyl group, and the cycloalkyl group in R²⁰¹ to R²⁰³ may have are each preferably an alkyl group (having, for example, 1 to 15 carbon atoms), a cycloalkyl group (having, for example, 3 to 15 carbon atoms), an aryl group (having, for example, 6 to 14 carbon atoms), an alkoxy group (having, for example, 1 to 15 carbon atoms), a cycloalkylalkoxy group (having, for example, 1 to 15 carbon atoms), a halogen atom (for example, fluorine or iodine), a hydroxy group, a carboxyl group, an ester group, a sulfanyl group, a sulfinyl group, a sulfonyl group, an alkylthio group, or a phenylthio group.

If possible, the substituents each may further have a substituent. It is also preferable that the alkyl group have a halogen atom as a substituent to form a halogenated alkyl group such as a trifluoromethyl group.

It is also preferable that any combination of the above substituents form an acid-decomposable group.

Next, the cation (ZaI-2) will be described.

The cation (ZaI-2) is a cation in which R²⁰¹ to R²⁰³ in the formula (ZaI) each independently represent an organic group having no aromatic ring. The aromatic ring also encompasses an aromatic ring including a heteroatom.

The number of carbon atoms of each of the organic groups having no aromatic ring and serving as R²⁰¹ to R²⁰³ is preferably 1 to 30, more preferably 1 to 20.

R²⁰¹ to R²⁰³ are each independently preferably an alkyl group, a cycloalkyl group, an allyl group, or a vinyl group, more preferably a linear or branched 2-oxoalkyl group, a 2-oxocycloalkyl group, or an alkoxycarbonylmethyl group, still more preferably a linear or branched 2-oxoalkyl group.

Examples of the alkyl group and the cycloalkyl group in R²⁰¹ to R²⁰³ include linear alkyl groups having 1 to 10 carbon atoms and branched alkyl groups having 3 to 10 carbon atoms (such as a methyl group, an ethyl group, a propyl group, a butyl group, and a pentyl group), and cycloalkyl groups having 3 to 10 carbon atoms (such as a cyclopentyl group, a cyclohexyl group, and a norbornyl group).

R²⁰¹ to R²⁰³ may be further substituted with a halogen atom, an alkoxy group (having, for example, 1 to 5 carbon atoms), a hydroxy group, a cyano group, or a nitro group.

It is also preferable that the substituents in R²⁰¹ to R²⁰³ each independently form an acid-decomposable group by any combination of substituents.

Next, the cation (ZaI-3b) will be described.

The cation (ZaI-3b) is a cation represented by a formula (ZaI-3b) below.

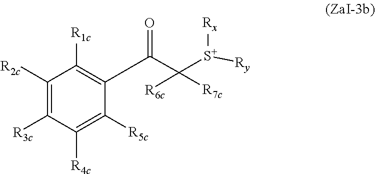

In the formula (ZaI-3b), R_{1c} to R_{5c}, each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxy group, a nitro group, an alkylthio group, or an arylthio group.
R_{6c} and R_{7c} each independently represent a hydrogen atom, an alkyl group (such as a t-butyl group), a cycloalkyl group, a halogen atom, a cyano group, or an aryl group.
Rₓ and R_{y} each independently represent an alkyl group, a cycloalkyl group, a 2-oxoalkyl group, a 2-oxocycloalkyl group, an alkoxycarbonylalkyl group, an allyl group, or a vinyl group.

It is also preferable that substituents in R_{1c} to R_{7c} and Rₓ and R_{y} each independently form an acid-decomposable group by any combination of substituents.

Any two or more of R_{1c} to R_{5c}, R_{5c} and R_{6c}, R_{6c} and R_{7c}, R_{5c}, and Rₓ, and Rₓ and R_{y} may be individually bonded together to form rings, and these rings may each independently include an oxygen atom, a sulfur atom, a ketone group, an ester bond, or an amide bond.

The rings each may be an aromatic or non-aromatic hydrocarbon ring, an aromatic or non-aromatic heterocycle, or a polycyclic fused ring formed by a combination of two or more of these rings. The ring may be a three- to ten-membered ring, and is preferably a four- to eight-membered ring, more preferably a five- or six-membered ring.

Examples of the groups formed by bonding together any two or more of R_{1c} to R_{5c}, R_{6c} and R_{7c}, and Rₓ and R_{y} include alkylene groups such as a butylene group and a pentylene group. A methylene group in such an alkylene group may be substituted with a heteroatom such as an oxygen atom.

The groups formed by bonding together R_{5c} and R_{6c}, and R_{5c} and Rₓ are each preferably a single bond or an alkylene group. Examples of the alkylene group include a methylene group and an ethylene group.

R_{1c} to R_{5c}, R_{6c}, R_{7c}, Rₓ, R_{y}, and the rings formed by individually bonding together any two or more of R_{1c} to R_{5c}, R_{5c} and R_{6c}, R_{6c} and R_{7c}, R_{5c} and Rₓ, and Rₓ and R_{y} may have a substituent.

Next, the cation (ZaI-4b) will be described.

The cation (ZaI-4b) is a cation represented by a formula (ZaI-4b) below.

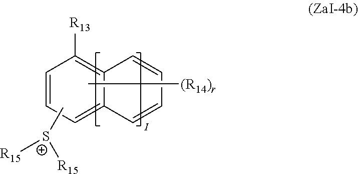

In the formula (ZaI-4b), 1 represents an integer of 0 to 2, and r represents an integer of 0 to 8.

R₁₃ represents a hydrogen atom, a halogen atom (for example, a fluorine atom or an iodine atom), a hydroxy group, an alkyl group, a halogenated alkyl group, an alkoxy group, a carboxyl group, an alkoxycarbonyl group, or a group including a cycloalkyl group (which may be a cycloalkyl group itself or a group including a cycloalkyl group as a part thereof). These groups may have a substituent.

R₁₄ represents a hydroxy group, a halogen atom (for example, a fluorine atom or an iodine atom), an alkyl group, a halogenated alkyl group, an alkoxy group, an alkoxycarbonyl group, an alkylcarbonyl group, an alkylsulfonyl group, a cycloalkylsulfonyl group, or a group including a cycloalkyl group (which may be a cycloalkyl group itself or a group including a cycloalkyl group as a part thereof). These groups may have a substituent. When a plurality of R₁₄'s are present, the plurality of R₁₄'s each independently represent the above-mentioned group, for example, a hydroxy group.

R₁₅'s each independently represent an alkyl group, a cycloalkyl group, or a naphthyl group. Two R₁₅'s may be bonded together to form a ring. When two R₁₅'s are bonded together to form a ring, the ring skeleton may include a heteroatom such as an oxygen atom or a nitrogen atom.

In one embodiment, two R₁₅'s are preferably alkylene groups and bonded together to form a ring structure. Note that the alkyl group, the cycloalkyl group, the naphthyl group, and the ring formed by bonding two R₁₅'s together may have a substituent.

In the formula (ZaI-4b), the alkyl groups in R₁₃, R₁₄, and R₁₅'s may be linear or branched. The number of carbon atoms of each of the alkyl groups is preferably 1 to 10. The alkyl group is preferably a methyl group, an ethyl group, a n-butyl group, a t-butyl group, or the like.

It is also preferable that substituents in R₁₃ to R₁₅ each independently form an acid-decomposable group by any combination of substituents.

Next, the formula (ZaII) will be described.

In the formula (ZaII), R²⁰⁴ and R²⁰⁵ each independently represent an aryl group, an alkyl group, or a cycloalkyl group.

The aryl group in R²⁰⁴ and R²⁰⁵ is preferably a phenyl group or a naphthyl group, more preferably a phenyl group. The aryl group in R²⁰⁴ and R²⁰⁵ may be an aryl group having a heterocycle having an oxygen atom, a nitrogen atom, a sulfur atom, or the like. Examples of the skeleton of the aryl group having a heterocycle include pyrrole, furan, thiophene, indole, benzofuran, and benzothiophene.

The alkyl group and the cycloalkyl group in R²⁰⁴ and R²⁰⁵ are preferably a linear alkyl group having 1 to 10 carbon atoms or a branched alkyl group having 3 to 10 carbon atoms (for example, a methyl group, an ethyl group, a propyl group, a butyl group, or a pentyl group), or a cycloalkyl group having 3 to 10 carbon atoms (for example, a cyclopentyl group, a cyclohexyl group, or a norbornyl group).

The aryl group, the alkyl group, and the cycloalkyl group in R²⁰⁴ and R²⁰⁵ may each independently have a substituent. Examples of the substituent that the aryl group, the alkyl group, and the cycloalkyl group in R²⁰⁴ and R²⁰⁵ may have include alkyl groups (having, for example, 1 to 15 carbon atoms), cycloalkyl groups (having, for example, 3 to 15 carbon atoms), aryl groups (having, for example, 6 to 15 carbon atoms), alkoxy groups (having, for example, 1 to 15 carbon atoms), halogen atoms, a hydroxy group, and a phenylthio group. It is also preferable that the substituents in R²⁰⁴ and R²⁰⁵ each independently form an acid-decomposable group by any combination of substituents.

Specific examples of the cations represented by M₁⁺ and M₂⁺ are shown below, but are not limited thereto.

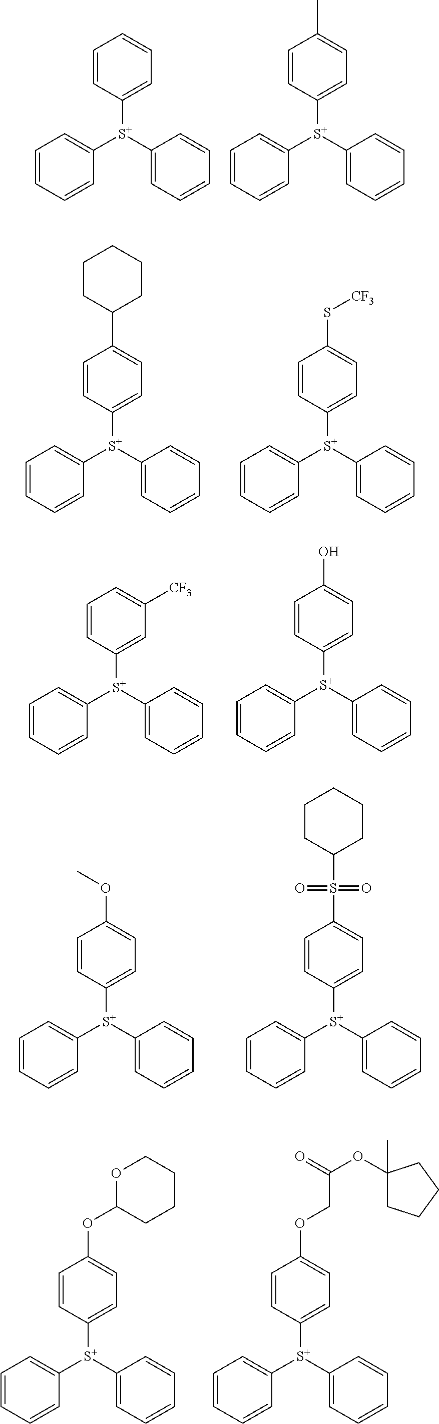

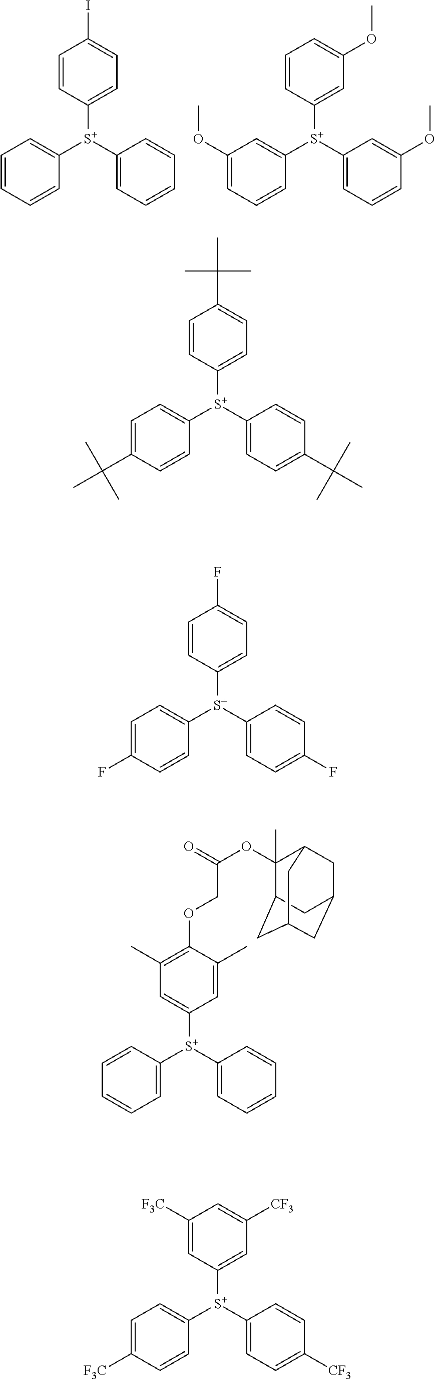 [SMILES: Ic1ccc([S+](c2ccccc2)c2ccccc2)cc1 ; COc1cccc([S+](c2cccc(OC)c2)c2cccc(OC)c2)c1 ; CC(C)(C)c1ccc([S+](c2ccc(C(C)(C)C)cc2)c2ccc(C(C)(C)C)cc2)cc1 ; Fc1ccc([S+](c2ccc(F)cc2)c2ccc(F)cc2)cc1 ; Cc1cc([S+](c2ccccc2)c2ccccc2)cc(C)c1OCC(=O)OC1(C)C2CC3CC(C2)CC1C3 ; FC(F)(F)c1ccc([S+](c2ccc(C(F)(F)F)cc2)c2cc(C(F)(F)F)cc(C(F)(F)F)c2)cc1]

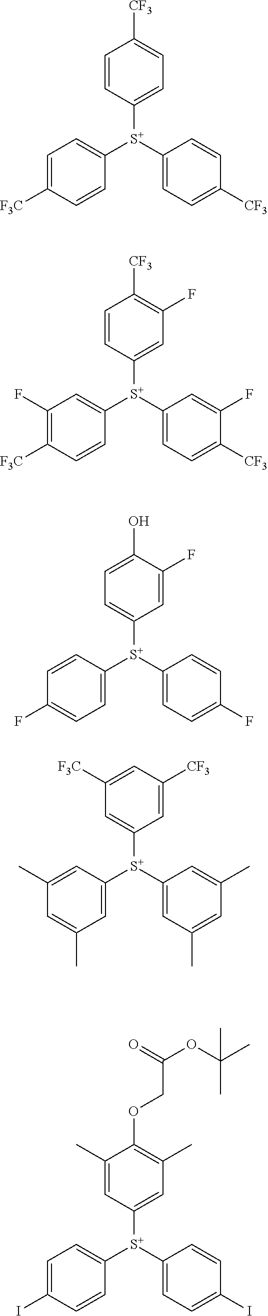

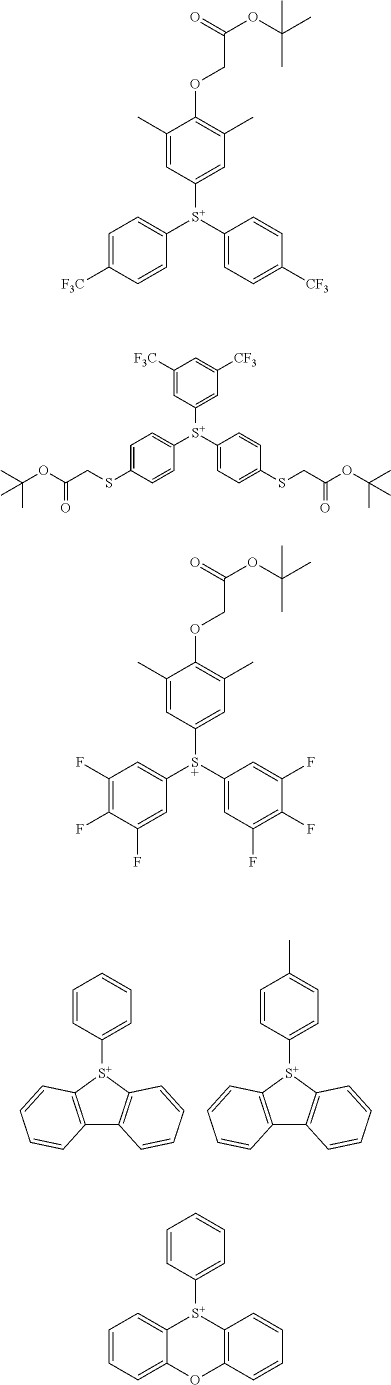 [SMILES: Cc1cc([S+](c2ccc(C(F)(F)F)cc2)c2ccc(C(F)(F)F)cc2)cc(C)c1OCC(=O)OC(C)(C)C ; CC(C)(C)OC(=O)CSc1ccc([S+](c2ccc(SCC(=O)OC(C)(C)C)cc2)c2cc(C(F)(F)F)cc(C(F)(F)F)c2)cc1 ; Cc1cc([S+](c2cc(F)c(F)c(F)c2)c2cc(F)c(F)c(F)c2)cc(C)c1OCC(=O)OC(C)(C)C ; c1ccc(-[s+]2c3ccccc3c3ccccc32)cc1 ; Cc1ccc(-[s+]2c3ccccc3c3ccccc32)cc1 ; c1ccc([S+]2c3ccccc3Oc3ccccc32)cc1]

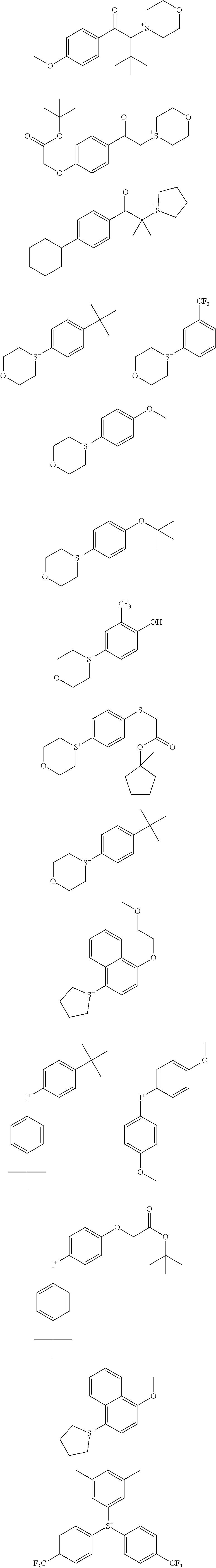

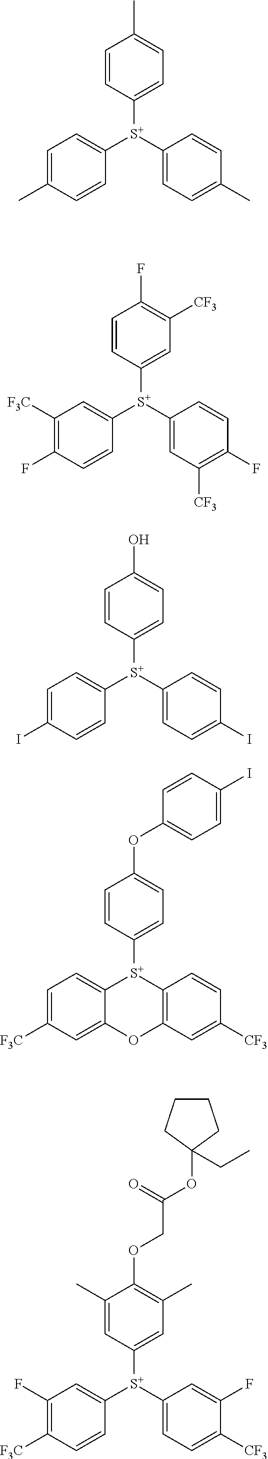

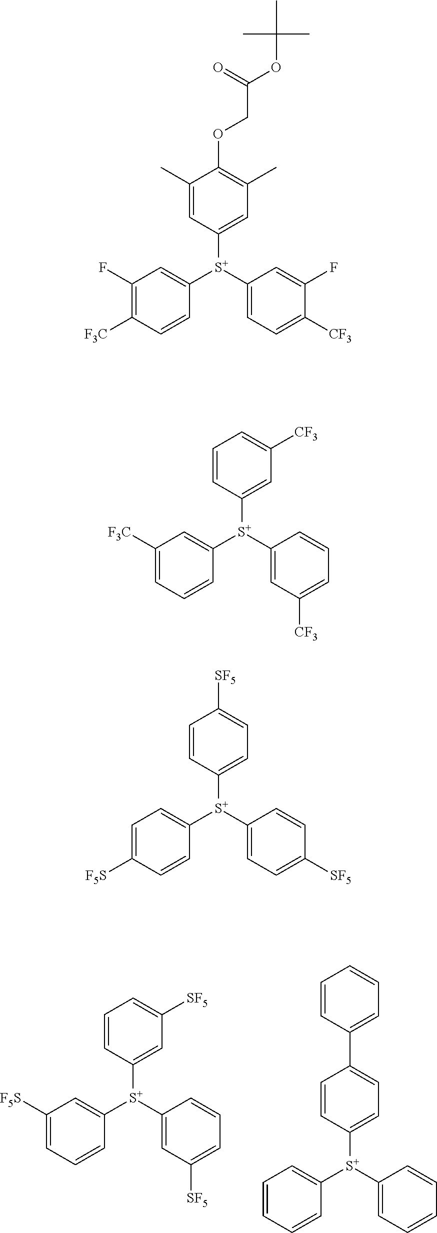

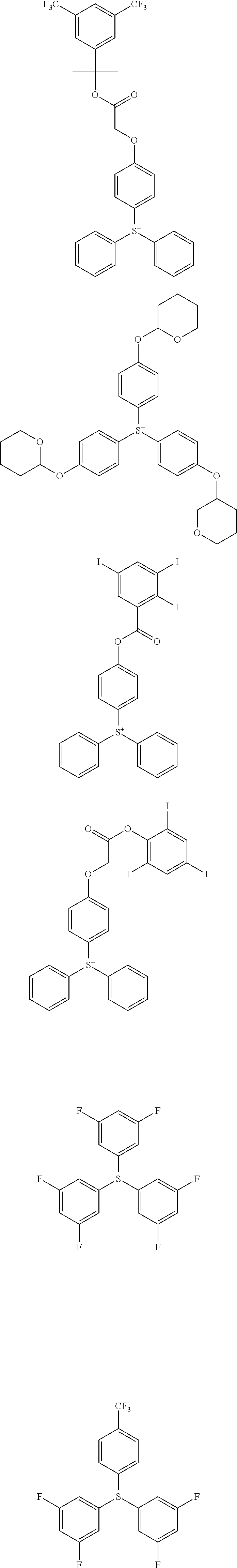 [SMILES: CC(C)(OC(=O)COc1ccc([S+](c2ccccc2)c2ccccc2)cc1)c1cc(C(F)(F)F)cc(C(F)(F)F)c1 ; c1cc([S+](c2ccc(OC3CCCCO3)cc2)c2ccc(OC3CCCCO3)cc2)ccc1OC1CCCOC1 ; O=C(Oc1ccc([S+](c2ccccc2)c2ccccc2)cc1)c1cc(I)cc(I)c1I ; O=C(COc1ccc([S+](c2ccccc2)c2ccccc2)cc1)Oc1c(I)cc(I)cc1I ; Fc1cc(F)cc([S+](c2cc(F)cc(F)c2)c2cc(F)cc(F)c2)c1 ; Fc1cc(F)cc([S+](c2ccc(C(F)(F)F)cc2)c2cc(F)cc(F)c2)c1]

The compound (B) is not particularly limited, but in the case where the compound (B) is a compound having one structural moiety X and one structural moiety Y, the compound may be, for example, a compound represented by a general formula (B-1) below.

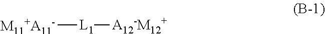

In the general formula (B-1), “M₁₁⁺A₁₁⁻” and “A₁₂⁻M₁₂⁺” correspond to the structural moiety X and the structural moiety Y, respectively. The compound represented by the general formula (B-1) generates an acid represented by HA₁₁-L1-A₁₂H upon irradiation with actinic rays or radiation. That is, “M₁₁⁺A₁₁⁻” forms a first acidic moiety represented by HA₁₁, and “A₁₂⁻M₁₂⁺” forms a second acidic moiety represented by HA₁₂ having a structure different from the first acidic moiety.

In the general formula (B-1), M₁₁⁺ and M₁₂⁺ each independently represent an organic cation.

A₁₁⁻ and A₁₂⁻ each independently represent an anionic functional group. However, A₁₂⁻ represents a structure different from the anionic functional group represented by A₁₁⁻.

L₁ represents a single bond or a divalent linking group.

However, in a compound PIa (HA₁₁-L₁-A₁₂H) obtained by replacing the organic cations represented by M₁₁⁺ and M₁₂⁺ in the general formula (B-1) with H⁺, the acid dissociation constant a2 derived from the acidic moiety represented by A₁₂H is larger than the acid dissociation constant a1 derived from the acidic moiety represented by HA₁₁. Preferred values of the acid dissociation constant a1 and the acid dissociation constant a2 are the same as those described above.

In the compound represented by the general formula (B-1), A₁₂⁻ is an anionic functional group including a partial structure represented by the formula (1) above.

In the general formula (B-1), examples of the organic cations represented by M₁₁⁺ and M₁₂⁺ include the cations described as M₁⁺ and M₂⁺, and preferred examples thereof are also the same as those of M₁⁺ and M₂⁺.

In the general formula (B-1), the anionic functional group represented by A₁₂⁻ is an anionic functional group including a partial structure represented by the formula (1) and is preferably an anionic functional group represented by a formula (1a) below.

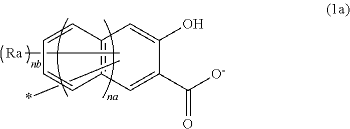

In the formula (1a),
Ra represents a monovalent substituent.
* represents a bonding site to L₁.
na represents 0 or 1.
nb represents an integer of 0 to (4+2×na−1).
When nb is an integer of 2 or more, a plurality of Ra's may be the same or different.

In the formula (1a), Ra represents a monovalent substituent. The monovalent substituent may be, for example, a halogen atom, a hydroxy group, an alkyl group, an alkoxy group, an alkoxycarbonyl group, an aryl group, an aryloxy group, or an aryloxycarbonyl group.

Examples of the halogen atom represented by Ra include a fluorine atom, a chlorine atom, a bromine atom, and an iodine atom. The halogen atom is preferably a fluorine atom or an iodine atom, more preferably an iodine atom.

Examples of the alkyl group represented by Ra include linear or branched alkyl groups having 1 to 15 carbon atoms, such as a methyl group, an ethyl group, a n-propyl group, an isopropyl group, a n-butyl group, an isobutyl group, and a t-butyl group.

Examples of the alkyl group in the alkoxy group and the alkoxycarbonyl group represented by Ra include the same groups as the above alkyl groups serving as Ra.

Examples of the aryl group represented by Ra include aryl groups having 6 to 15 carbon atoms, such as a phenyl group and a naphthyl group.

Examples of the aryl group in the aryloxy group and the aryloxycarbonyl group represented by Ra include the same groups as the above aryl groups serving as Ra.

The monovalent substituent may further have a substituent. The additional substituent may be a halogen atom, a hydroxy group, an alkyl group, a cycloalkyl group, an alkoxy group, an alkoxycarbonyl group, an aryl group, an aryloxy group, or an aryloxycarbonyl group. The cycloalkyl group may have a heteroatom such as an oxygen atom as a ring member atom. The halogen atom is preferably a fluorine atom, a chlorine atom, a bromine atom, or an iodine atom, more preferably a fluorine atom or an iodine atom, still more preferably an iodine atom. The iodine atom is preferably directly bonded to the aromatic ring.

The additional substituent may further have a substituent. The substituent that the additional substituent may further have may be, for example, a halogen atom. The halogen atom is preferably a fluorine atom, a chlorine atom, a bromine atom, or an iodine atom, more preferably a fluorine atom or an iodine atom, still more preferably an iodine atom. The iodine atom is preferably directly bonded to the aromatic ring.

In the formula (1a), na represents 0 or 1, and is preferably 0.

nb represents an integer of 0 to (4+2×na−1), and is preferably 0 to 2.

In the general formula (B-1), the anionic functional group represented by A₁₁⁻ may be an anionic functional group including a partial structure selected from the group consisting of the formulae (CC-1) to (CC-7) above, and is preferably an anionic functional group including a partial structure represented by any one of the formulae (2) to (5) above, more preferably an anionic functional group selected from the group consisting of the formulae (2) and (3) above and formulae (4a), (5a), and (5b) below.

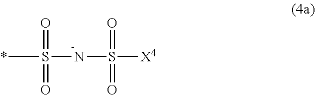

In the formula (4a), X⁴ represents an organic group. * represents a bonding site to L₁.

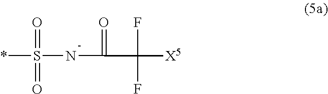

In the formula (5a), X⁵ represents a halogen atom or an organic group. * represents a bonding site to L₁.

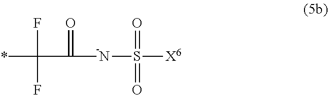

In the formula (5b), X⁶ represents an organic group. * represents a bonding site to L₁.

In the formula (4a), X⁴ represents an organic group.

The organic group represented by X⁴ is preferably an alkyl group, a cycloalkyl group, or an aryl group.

The number of carbon atoms of the alkyl group represented by X⁴ is preferably 1 to 15, more preferably 1 to 10.

The alkyl group may have a substituent. The substituent is preferably a fluorine atom or a cyano group. When the alkyl group has a fluorine atom as a substituent, the alkyl group may be a perfluoroalkyl group.

A carbon atom of the alkyl group may be substituted with a carbonyl group.

The number of carbon atoms of the cycloalkyl group represented by X⁴ is preferably 3 to 15, more preferably 3 to 10.

The cycloalkyl group may have a substituent. Examples of the substituent include the same groups as the substituents that the alkyl group may have.

The aryl group represented by X⁴ is preferably a phenyl group or a naphthyl group, more preferably a phenyl group.

The aryl group may have a substituent. The substituent is preferably a fluorine atom, a perfluoroalkyl group (for example, preferably having 1 to 10 carbon atoms, more preferably having 1 to 6 carbon atoms), or a cyano group.

In the formula (5a), X⁵ represents a halogen atom or an organic group.

The halogen atom represented by X⁵ is preferably a fluorine atom.

Examples of the organic group represented by X⁵ include the organic groups represented by X⁴ in the formula (4a), and preferred examples thereof are also the same as those of the organic groups represented by X⁴.

In the formula (5b), X⁶ represents an organic group.

Examples of the organic group represented by X⁶ include the organic groups represented by X⁴ in the formula (4a), and preferred examples thereof are also the same as those of the organic groups represented by X⁴.

In the general formula (B-1), L₁ represents a single bond or a divalent linking group.

Examples of the divalent linking group represented by L₁ include, but are not particularly limited to, —CO—, —NR—, —CO₂—, —O—, —S—, —SO—, —SO₂—, alkylene groups (which preferably have 1 to 6 carbon atoms and may be linear or branched), cycloalkylene groups (preferably having 3 to 15 carbon atoms), alkenylene groups (preferably having 2 to 6 carbon atoms), divalent aliphatic heterocyclic groups (preferably five- to ten-membered rings, more preferably five- to seven-membered rings, still more preferably five- or six-membered rings each having at least one N atom, O atom, S atom, or Se atom in the ring structure), divalent aromatic heterocyclic groups (preferably five- to ten-membered rings, more preferably five- to seven-membered rings, still more preferably five- or six-membered rings each having at least one N atom, O atom, S atom, or Se atom in the ring structure), divalent aromatic hydrocarbon ring groups (preferably six- to ten-membered rings, more preferably six-membered rings), and divalent linking groups provided by combining a plurality of these. R above is, for example, a hydrogen atom or a monovalent organic group. The monovalent organic group is not particularly limited, but is preferably, for example, an alkyl group (preferably having 1 to 6 carbon atoms).

The alkylene groups, the cycloalkylene groups, the alkenylene groups, the divalent aliphatic heterocyclic groups, the divalent aromatic heterocyclic groups, and the divalent aromatic hydrocarbon ring groups may be substituted with a substituent. The substituent is, for example, a halogen atom (preferably a fluorine atom).

In the case where the compound (B) is a compound having two or more structural moieties X and at least one structural moiety Y, the compound (B) may be, for example, a compound represented by a general formula (B-2) below.

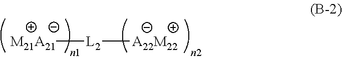

In the general formula (B-2), “M₂₁⁺A₂₁⁻” and “A₂₂⁻M₂₂⁺” correspond to the structural moiety X and the structural moiety Y, respectively. The compound represented by the general formula (B-2) generates an acid represented by the following general formula (B-2-1) upon irradiation with actinic rays or radiation. That is, “M₂₁⁺A₂₁⁻” forms a first acidic moiety represented by HA₂₁, and “A₂₂⁻M₂₂⁺” forms a second acidic moiety represented by HA₂₂ having a structure different from the first acidic moiety.

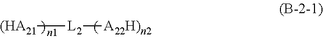

In the general formula (B-2), M₂₁⁺ and M₂₂⁺ each independently represent an organic cation.

A₂₁⁻ and A₂₂⁻ each independently represent an anionic functional group. However, A₂₂⁻ represents a structure different from the anionic functional group represented by A₂₁⁻.
L₂ represents an (n1+n2)-valent organic group.
n1 represents an integer of 2 or more.
n2 represents an integer of 1 or more.

However, in a compound PIIa obtained by replacing the organic cations represented by M₂₁⁺ and M₂₂⁺ in the general formula (B-2) with H⁺ (corresponding to the compound represented by the general formula (B-2-1)), the acid dissociation constant a2 derived from the acidic moiety represented by A₂₂H is larger than the acid dissociation constant a1 derived from the acidic moiety represented by HA₂₁. Preferred values of the acid dissociation constant a1 and the acid dissociation constant a2 are the same as those described above.

In the compound represented by the general formula (B-2), A₂₂⁻ is an anionic functional group including a partial structure represented by the formula (1) above.

In the general formula (B-2), M₂₁⁺, M₂₂⁺, A₂₁⁻, and A₂₂⁻ have the same definitions as M₁₁⁺, M₁₂⁺, A₁₁⁻, and A₁₂⁻ in the general formula (B-1) described above, respectively, and preferred embodiments thereof are also the same as those of M₁₁⁺, M₁₂⁺, A₁₁⁻, and A₁₂⁻.

In the general formula (B-2), n1 M₂₁⁺'s represent the same group, and n1 A₂₁⁻'s represent the same group.

In the general formula (B-2), the (n1+n2)-valent organic group represented by L₂ is not particularly limited and may be, for example, a group represented by (A1) or (A2) below. In (A1) and (A2) below, at least two of * represent bonding sites to A₂₁⁻, and at least one of * represents a bonding site to A₂₂⁻.

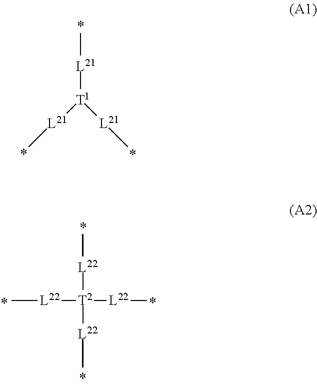

In the (A1) and (A2), T¹ represents a trivalent hydrocarbon ring group or a trivalent heterocyclic group, and T² represents a carbon atom, a tetravalent hydrocarbon ring group, or a tetravalent heterocyclic group.

The hydrocarbon ring group may be an aromatic hydrocarbon ring group or an aliphatic hydrocarbon ring group. The number of carbon atoms included in the hydrocarbon ring group is preferably 6 to 18, more preferably 6 to 14.

The heterocyclic group may be an aromatic heterocyclic group or an aliphatic heterocyclic group. The heterocycle is preferably a five- to ten-membered ring, more preferably a five- to seven-membered ring, still more preferably a five- or six-membered ring having at least one N atom, O atom, S atom, or Se atom in the ring structure.

In the (A1) and (A2), L²¹ and L²² each independently represent a single bond or a divalent linking group.

The divalent linking group represented by L²¹ or L²² has the same definition as the divalent linking group represented by L₁ in the general formula (B-1), and preferred embodiments thereof are also the same as those of the divalent linking group represented by L₁.

n1 represents an integer of 2 or more. The upper limit is not particularly limited, but is, for example, 6 or less, preferably 4 or less, more preferably 3 or less.

n2 represents an integer of 1 or more. The upper limit is not particularly limited, but is, for example, 3 or less, preferably 2 or less.

In the case where the compound (B) is a compound having two or more structural moieties X and a structural moiety Y, for example, a compound represented by a general formula (B-3) below is also preferable.

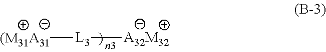

In the general formula (B-3), “M₃₁⁺A₃₁⁻” and “A₃₂⁻M₃₂⁺” correspond to the structural moiety X and the structural moiety Y, respectively. The compound represented by the general formula (B-3) generates an acid represented by the following general formula (B-3-1) upon irradiation with actinic rays or radiation. That is, “M₃₁⁺A₃₁⁻” forms a first acidic moiety represented by HA₃₁, and “A₃₂⁻M₃₂⁺” forms a second acidic moiety represented by HA₃₂ having a structure different from the first acidic moiety.

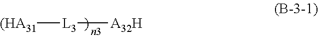

In the general formula (B-3), M₃₁⁺ and M₃₂⁺ each independently represent an organic cation.

A₃₁⁻ and A₃₂⁻ each independently represent an anionic functional group. However, A₃₂⁻ represents a structure different from the anionic functional group represented by A₃₁⁻.
L₃ represents a single bond or a divalent linking group.
n3 represents an integer of 2 or more.

However, in a compound PIIIa obtained by replacing the organic cations represented by M₃₁⁺ and M₃₂⁺ in the general formula (B-3) with H⁺ (corresponding to the compound represented by the general formula (B-3-1)), the acid dissociation constant a2 derived from the acidic moiety represented by A₃₂H is larger than the acid dissociation constant a1 derived from the acidic moiety represented by HA₃₁. Preferred values of the acid dissociation constant a1 and the acid dissociation constant a2 are the same as those described above.

In the compound represented by the general formula (B-3), A₃₂⁻ is an anionic functional group including a partial structure represented by the formula (1) above.

In the general formula (B-3), M₃₁⁺, M₃₂⁺, and A₃₁⁻ have the same definitions as M₁₁⁺, M₁₂⁺, and A₁₁⁻ in the general formula (B-1), respectively, and preferred embodiments thereof are also the same as those of M₁₁⁺, M₁₂⁺, and A₁₁⁻.

In the general formula (B-3), the anionic functional group represented by A₃₂⁻ is an anionic functional group including a partial structure represented by the formula (1), and is preferably an anionic functional group represented by a formula (1b) below.

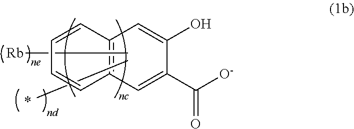

In the formula (1b),
Rb represents a monovalent substituent.
* represents a bonding site to L₃.
nc represents 0 or 1.
nd represents an integer of 2 to 3.
ne represents an integer of 0 to (4+2×nc−nd).
When ne is an integer of 2 or more, a plurality of Rb's may be the same or different.

In the formula (1b), Rb has the same definition as Ra in the formula (1a), and preferred examples thereof are also the same as those of Ra.

In the formula (1b), nc represents 0 or 1, and is preferably 0.
nd represents an integer of 2 to 3, and is preferably 2.
ne represents an integer of 0 to (4+2×nc−nd), and is preferably 0 to 2.

In the general formula (B-3), the divalent linking group represented by L₃ may be the divalent linking group serving as L₁ in the general formula (B-1).

Specific examples of the structure other than the cationic moiety in the compound (B) are shown below, but the present invention is not limited thereto.

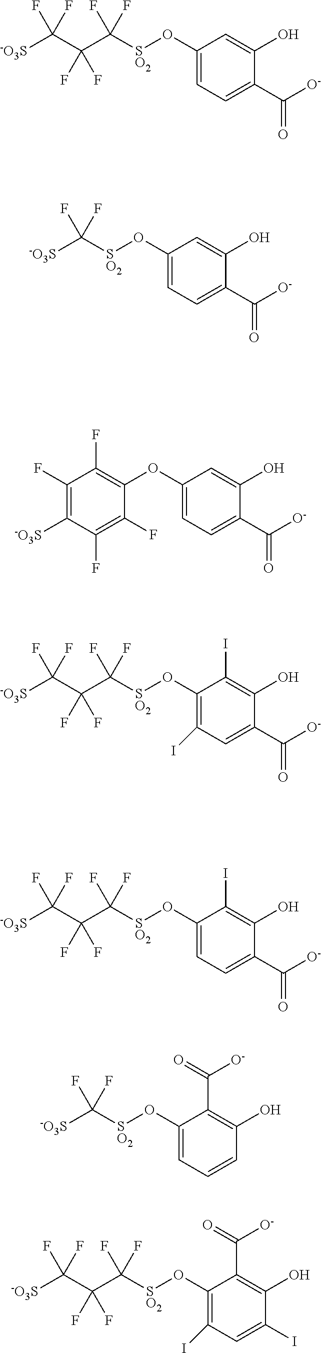

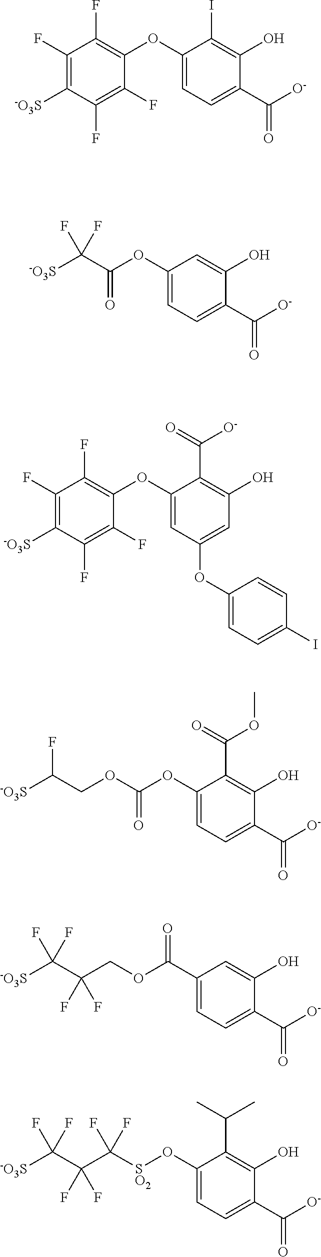

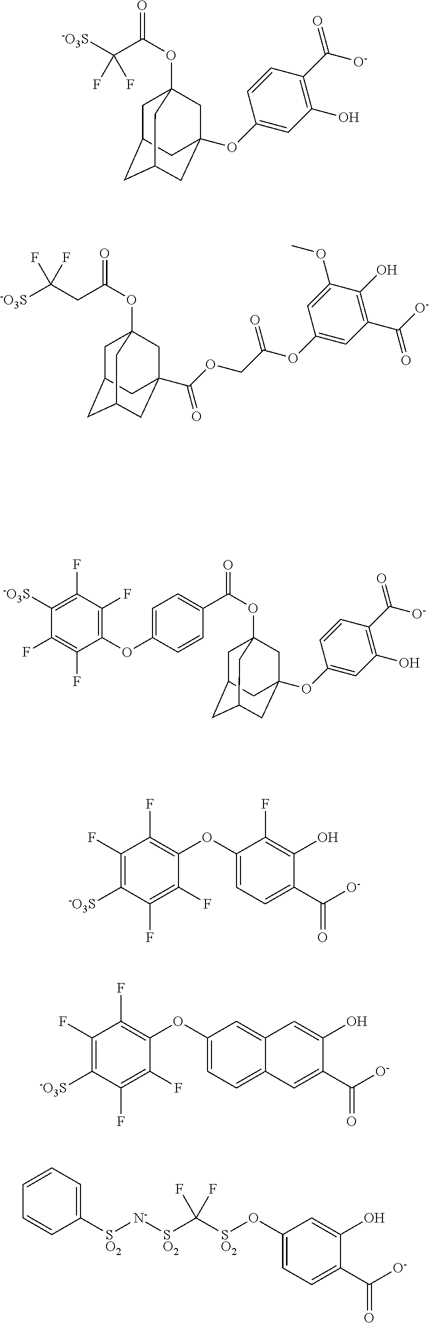

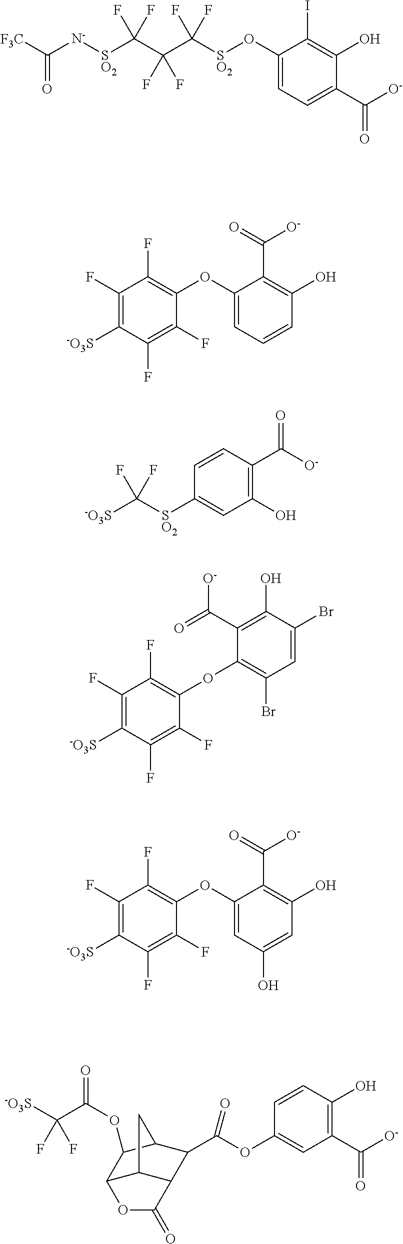

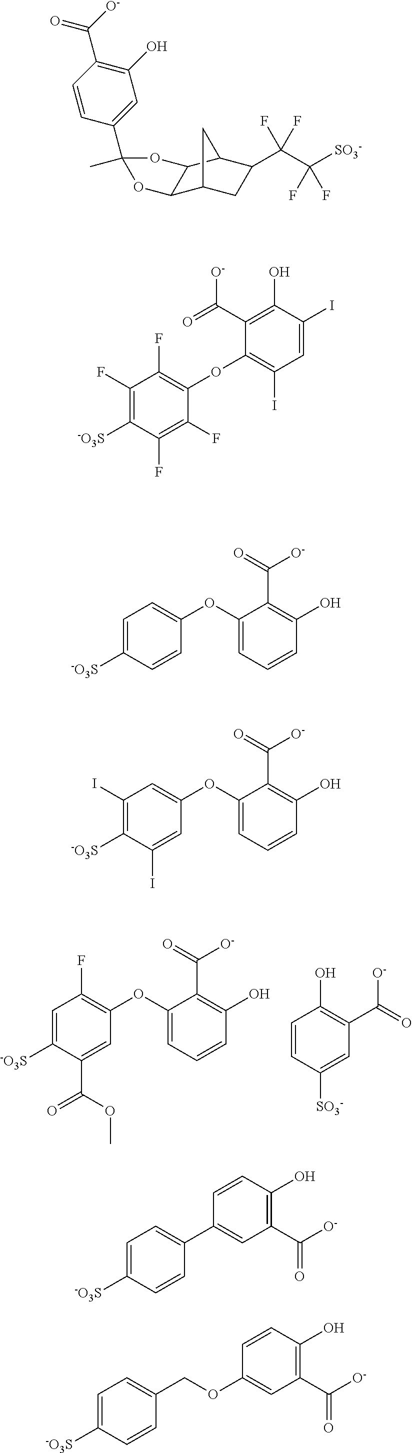

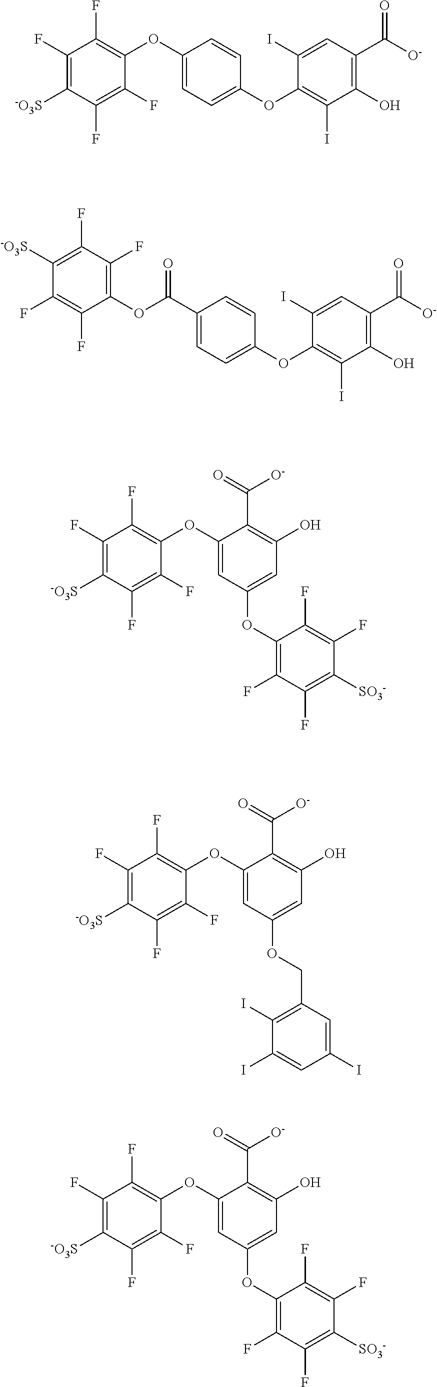

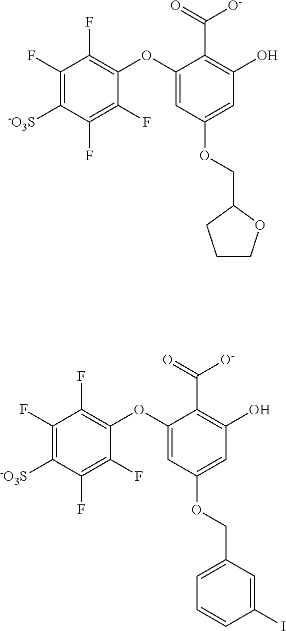

The content of the compound (B) in the composition of the present invention is not particularly limited, but is preferably 90 mass % or less, more preferably 80 mass % or less, still more preferably 70 mass % or less relative to the total solid contents of the composition of the present invention.

The content of the compound (B) in the composition of the present invention is not particularly limited, but is preferably 5 mass % or more, more preferably 10 mass % or more, still more preferably 15 mass % or more relative to the total solid contents of the composition of the present invention.

Such compounds (B) may be used alone or in combination of two or more thereof.

### [Acid-Decomposable Resin (A)]

The composition of the present invention includes an acid-decomposable resin (A) (also referred to as a “resin (A)”).

The resin (A) is a resin whose polarity is increased by the action of an acid.

The resin (A) typically includes a group that is decomposed by the action of an acid to undergo an increase in polarity (also referred to as an “acid-decomposable group”), and preferably includes a repeating unit having an acid-decomposable group. In the case where the resin (A) has an acid-decomposable group, in the pattern forming method in the present specification, typically, when an alkali developer is used as the developer, a positive-type pattern is suitably formed, and when an organic-based developer is used as the developer, a negative-type pattern is suitably formed.

The repeating unit having an acid-decomposable group is preferably, in addition to the repeating unit having an acid-decomposable group, a repeating unit having an acid-decomposable group including an unsaturated bond.

### (Repeating Unit Having Acid-Decomposable Group)

The acid-decomposable group refers to a group that is decomposed by the action of an acid to generate a polar group. The acid-decomposable group preferably has a structure in which the polar group is protected with a group (leaving group) that leaves due to the action of an acid. That is, the resin (A) has a repeating unit having a group that is decomposed by the action of an acid to generate a polar group. In the resin having this repeating unit, the polarity is increased by the action of an acid, and thus the degree of solubility in an alkali developer increases, and the degree of solubility in an organic solvent decreases.

The polar group is preferably an alkali-soluble group, and examples thereof include acidic groups such as a carboxyl group, phenolic hydroxy groups, fluorinated alcohol groups, a sulfonic group, a phosphate group, a sulfonamide group, a sulfonylimide group, (alkylsulfonyl)(alkylcarbonyl)methylene groups, (alkylsulfonyl)(alkylcarbonyl)imide groups, bis(alkylcarbonyl)methylene groups, bis(alkylcarbonyl)imide groups, bis(alkylsulfonyl)methylene groups, bis(alkylsulfonyl)imide groups, tris(alkylcarbonyl)methylene groups, and tris(alkylsulfonyl)methylene groups, and alcoholic hydroxy groups.

Of these, the polar group is preferably a carboxyl group, a phenolic hydroxy group, a fluorinated alcohol group (preferably a hexafluoroisopropanol group), or a sulfonic group.

Examples of the group that leaves due to the action of an acid include groups represented by formulae (Y1) to (Y4).

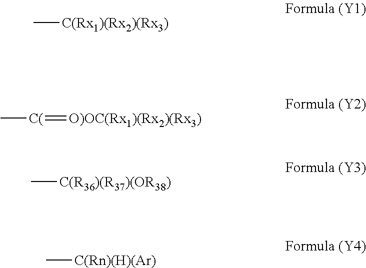

In the formula (Y1) and the formula (Y2), Rx₁ to Rx₃ each independently represent an alkyl group (linear or branched), a cycloalkyl group (monocyclic or polycyclic), an alkenyl group (linear or branched), or an aryl group (monocyclic or polycyclic). Note that, when Rx₁ to Rx₃ are all alkyl groups (linear or branched), at least two of Rx₁ to Rx₃ are preferably methyl groups.

In particular, it is preferable that Rx₁ to Rx₃ each independently represent a linear or branched alkyl group, and it is more preferable that Rx₁ to Rx₃ each independently represent a linear alkyl group.

Two of Rx₁ to Rx₃ may be bonded together to form a monocycle or a polycycle.

The alkyl group in Rx₁ to Rx₃ is preferably an alkyl group having 1 to 5 carbon atoms, such as a methyl group, an ethyl group, a n-propyl group, an isopropyl group, a n-butyl group, an isobutyl group, or a t-butyl group.

The cycloalkyl group in Rx₁ to Rx₃ is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group.

The aryl group in Rx₁ to Rx₃ is preferably an aryl group having 6 to 10 carbon atoms and may be, for example, a phenyl group, a naphthyl group, or an anthryl group.

The alkenyl group in Rx₁ to Rx₃ is preferably a vinyl group.

The ring formed by bonding two of Rx₁ to Rx₃ together is preferably a cycloalkyl group. The cycloalkyl group formed by bonding two of Rx₁ to Rx₃ together is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group, more preferably a monocyclic cycloalkyl group having 5 or 6 carbon atoms.

In the cycloalkyl group formed by bonding two of Rx₁ to Rx₃ together, one of methylene groups constituting the ring may be replaced by a heteroatom such as an oxygen atom, a group including a heteroatom, such as a carbonyl group, or a vinylidene group. In any of these cycloalkyl groups, one or more ethylene groups constituting the cycloalkane ring may each be replaced by a vinylene group.

In a preferred embodiment of the group represented by the formula (Y1) or the formula (Y2), for example, Rx₁ is a methyl group or an ethyl group, and Rx₂ and Rx₃ are bonded together to form the above-described cycloalkyl group.

When the actinic ray-sensitive or radiation-sensitive resin composition is, for example, a resist composition for EUV exposure, the alkyl group, the cycloalkyl group, the alkenyl group, and the aryl group represented by Rx₁ to Rx₃ and the ring formed by bonding two of Rx₁ to Rx₃ together also preferably further have, as a substituent, a fluorine atom or an iodine atom.

In the formula (Y3), R₃₆ to R₃₈ each independently represent a hydrogen atom or a monovalent organic group. R₃₇ and R₃₈ may be bonded together to form a ring. Examples of the monovalent organic group include alkyl groups, cycloalkyl groups, aryl groups, aralkyl groups, and alkenyl groups. It is also preferable that R₃₆ be a hydrogen atom.

The alkyl groups, cycloalkyl groups, aryl groups, and aralkyl groups each may include a heteroatom such as an oxygen atom and/or a group including a heteroatom, such as a carbonyl group. For example, in each of the above-mentioned alkyl groups, cycloalkyl groups, aryl groups, and aralkyl groups, one or more methylene groups may be substituted with a heteroatom such as an oxygen atom and/or a group including a heteroatom, such as a carbonyl group.

R₃₈ may be bonded to another substituent included in the main chain of the repeating unit to form a ring. The group formed by bonding R₃₈ and another substituent included in the main chain of the repeating unit is preferably an alkylene group such as a methylene group.

When the actinic ray-sensitive or radiation-sensitive resin composition is, for example, a resist composition for EUV exposure, the monovalent organic group represented by R₃₆ to R₃₈ and the ring formed by bonding R₃₇ and R₃₈ together also preferably further have, as a substituent, a fluorine atom or an iodine atom.

The formula (Y3) preferably represents a group represented by a formula (Y3-1) below.

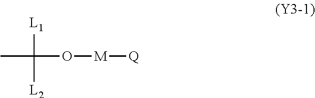

Here, L₁ and L₂ each independently represent a hydrogen atom, an alkyl group, a cycloalkyl group, an aryl group, or a combination thereof (for example, a combination of an alkyl group and an aryl group).

M represents a single bond or a divalent linking group.

Q represents an alkyl group that may include a heteroatom, a cycloalkyl group that may include a heteroatom, an aryl group that may include a heteroatom, an amino group, an ammonium group, a mercapto group, a cyano group, an aldehyde group, or a combination thereof (for example, a group provided by combining an alkyl group and a cycloalkyl group).

In the alkyl group and the cycloalkyl group, for example, one of methylene groups may be replaced by a heteroatom such as an oxygen atom or a group including a heteroatom, such as a carbonyl group.

Note that one of L₁ and L₂ is preferably a hydrogen atom, and the other is preferably an alkyl group, a cycloalkyl group, an aryl group, or a group that is a combination of an alkylene group and an aryl group.

At least two of Q, M, and L₁ may be bonded together to form a ring (preferably a five-membered or six-membered ring).

From the viewpoint of forming a finer pattern, L₂ is preferably a secondary or tertiary alkyl group, more preferably a tertiary alkyl group. Examples of the secondary alkyl group include an isopropyl group, a cyclohexyl group, and a norbornyl group, and examples of the tertiary alkyl group include a tert-butyl group and an adamantane group. In such embodiments, since Tg (glass transition temperature) and activation energy are high, the film hardness is ensured, and the occurrence of fogging can be suppressed.

When the actinic ray-sensitive or radiation-sensitive resin composition is, for example, a resist composition for EUV exposure, it is also preferable that the alkyl group, cycloalkyl group, aryl group, and combinations thereof, the groups being represented by L₁ and L₂, each further have a fluorine atom or an iodine atom as a substituent. It is also preferable that the alkyl group, the cycloalkyl group, the aryl group, and the aralkyl group include a heteroatom such as an oxygen atom in addition to a fluorine atom and an iodine atom. Specifically, in the alkyl group, the cycloalkyl group, the aryl group, and the aralkyl group, for example, one of methylene groups may be replaced by a heteroatom such as an oxygen atom or a group including a heteroatom, such as a carbonyl group.

When the actinic ray-sensitive or radiation-sensitive resin composition is, for example, a resist composition for EUV exposure, in the alkyl group that may include a heteroatom, the cycloalkyl group that may include a heteroatom, the aryl group that may include a heteroatom, the amino group, the ammonium group, the mercapto group, the cyano group, the aldehyde group, and a combination thereof, the groups being represented by Q, the heteroatom is also preferably a heteroatom selected from the group consisting of a fluorine atom, an iodine atom, and an oxygen atom.

In the formula (Y4), Ar represents an aromatic ring group. Rn represents an alkyl group, a cycloalkyl group, or an aryl group. Rn and Ar may be bonded together to form a non-aromatic ring. Ar is preferably an aryl group.

When the actinic ray-sensitive or radiation-sensitive resin composition is, for example, a resist composition for EUV exposure, it is also preferable that the aromatic ring group represented by Ar and the alkyl group, the cycloalkyl group, and the aryl group represented by Rn have a fluorine atom or an iodine atom as a substituent.

When, in the leaving group protecting the polar group, a non-aromatic ring is directly bonded to the polar group (or a residue thereof), it is also preferable that a ring member atom adjacent to the ring member atom directly bonded to the polar group (or a residue thereof) in the non-aromatic ring do not have a halogen atom such as a fluorine atom as a substituent, from the viewpoint that the repeating unit has excellent acid decomposability.

The group that leaves due to the action of an acid may be a 2-cyclopentenyl group having a substituent (e.g., an alkyl group), such as a 3-methyl-2-cyclopentenyl group; or a cyclohexyl group having a substituent (e.g., an alkyl group), such as a 1,1,4,4-tetramethylcyclohexyl group.

The repeating unit having an acid-decomposable group is also preferably a repeating unit represented by a formula (A).

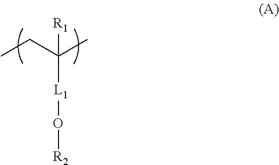

L₁ represents a divalent linking group that may have a fluorine atom or an iodine atom; R₁ represents a hydrogen atom, a fluorine atom, an iodine atom, an alkyl group that may have a fluorine atom or an iodine atom, or an aryl group that may have a fluorine atom or an iodine atom; and R₂ represents a leaving group that leaves due to the action of an acid and that may have a fluorine atom or an iodine atom. Note that at least one of L₁, R¹, or R₂ has a fluorine atom or an iodine atom.

Examples of the divalent linking group that may have a fluorine atom or an iodine atom and is represented by L₁ include —CO—, —O—, —S—, —SO—, —SO₂—, hydrocarbon groups that may have a fluorine atom or an iodine atom (for example, alkylene groups, cycloalkylene groups, alkenylene groups, and arylene groups), and linking groups provided by linking a plurality of these groups together. In particular, L₁ is preferably —CO—, an arylene group, or an -arylene group-fluorine or iodine atom-containing alkylene group-, more preferably —CO— or an -arylene group-fluorine or iodine atom-containing alkylene group-.

The arylene group is preferably a phenylene group.

The alkylene group may be linear or branched. The number of carbon atoms of the alkylene group is not particularly limited, but is preferably 1 to 10, more preferably 1 to 3.

The total number of fluorine atoms and iodine atoms included in the fluorine or iodine atom-containing alkylene group is not particularly limited, but is preferably 2 or more, more preferably 2 to 10, still more preferably 3 to 6.

The alkyl group represented by R₁ may be linear or branched. The number of carbon atoms of the alkyl group is not particularly limited, but is preferably 1 to 10, more preferably 1 to 3.

The total number of fluorine atoms and iodine atoms included in the fluorine or iodine atom-containing alkyl group represented by R₁ is not particularly limited, but is preferably 1 or more, more preferably 1 to 5, still more preferably 1 to 3.

The alkyl group represented by R₁ may include a heteroatom other than halogen atoms, such as an oxygen atom.

Examples of the leaving group that may have a fluorine atom or an iodine atom and is represented by R₂ include leaving groups that are represented by the formulae (Y1) to (Y4) and that have a fluorine atom or an iodine atom.

As the repeating unit having an acid-decomposable group, a repeating unit represented by a formula (AI) is also preferable.

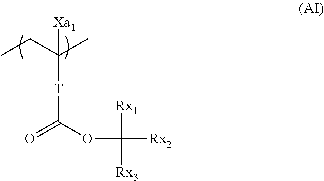

In the formula (AI), Xa₁ represents a hydrogen atom or an alkyl group that may have a substituent. T represents a single bond or a divalent linking group. Rx₁ to Rx₃ each independently represent an alkyl group (linear or branched), a cycloalkyl group (monocyclic or polycyclic), an alkenyl group (linear or branched), or an aryl (monocyclic or polycyclic) group. Note that, when Rx₁ to Rx₃ are all alkyl groups (linear or branched), at least two of Rx₁ to Rx₃ are preferably methyl groups.

Two of Rx₁ to Rx₃ may be bonded together to form a monocyclic or polycyclic ring (such as a monocyclic or polycyclic cycloalkyl group).

The alkyl group that may have a substituent and is represented by Xa₁ may be, for example, a methyl group or a group represented by —CH₂—R₁₁. R₁₁ represents a halogen atom (such as a fluorine atom), a hydroxy group, or a monovalent organic group. Examples of the monovalent organic group represented by R₁₁ include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. The monovalent organic group represented by R₁₁ is preferably an alkyl group having 3 or less carbon atoms, more preferably a methyl group. Xa₁ is preferably a hydrogen atom, a methyl group, a trifluoromethyl group, or a hydroxymethyl group.

Examples of the divalent linking group in T include alkylene groups, aromatic ring groups, a —COO-Rt- group, and an —O-Rt- group. In the formulae, Rt represents an alkylene group or a cycloalkylene group.

T is preferably a single bond or a —COO-Rt- group. When T represents a —COO-Rt- group, Rt is preferably an alkylene group having 1 to 5 carbon atoms, more preferably a —CH₂— group, a —(CH₂)₂— group, or a —(CH₂)₃— group.

The alkyl group in Rx₁ to Rx₃ is preferably an alkyl group having 1 to 4 carbon atoms, such as a methyl group, an ethyl group, a n-propyl group, an isopropyl group, a n-butyl group, an isobutyl group, or a t-butyl group.

The cycloalkyl group in Rx₁ to Rx₃ is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group.

The aryl group in Rx₁ to Rx₃ is preferably an aryl group having 6 to 10 carbon atoms and may be, for example, a phenyl group, a naphthyl group, or an anthryl group.

The alkenyl group in Rx₁ to Rx₃ is preferably a vinyl group.

The cycloalkyl group formed by bonding two of Rx₁ to Rx₃ together is preferably a monocyclic cycloalkyl group such as a cyclopentyl group or a cyclohexyl group. A polycyclic cycloalkyl group such as a norbornyl group, a tetracyclodecanyl group, a tetracyclododecanyl group, or an adamantyl group is also preferred. Of these, a monocyclic cycloalkyl group having 5 to 6 carbon atoms is preferred.

In the cycloalkyl group formed by bonding two of Rx₁ to Rx₃ together, for example, one of methylene groups constituting the ring may be replaced by a heteroatom such as an oxygen atom, a group including a heteroatom, such as a carbonyl group, or a vinylidene group. In any of these cycloalkyl groups, one or more ethylene groups constituting the cycloalkane ring may each be replaced by a vinylene group.

In a preferred embodiment of the repeating unit represented by the formula (AI), for example, Rx₁ is a methyl group or an ethyl group, and Rx₂ and Rx₃ are bonded together to form the above-described cycloalkyl group.

When any of the above groups has a substituent, examples of the substituent include alkyl groups (having 1 to 4 carbon atoms), halogen atoms, a hydroxy group, alkoxy groups (having 1 to 4 carbon atoms), a carboxyl group, and alkoxycarbonyl groups (having 2 to 6 carbon atoms). The number of carbon atoms in the substituent is preferably 8 or less.

As the repeating unit represented by the formula (AI), an acid-decomposable tertiary alkyl (meth)acrylate-based repeating unit (a repeating unit in which Xa₁ represents a hydrogen atom or a methyl group, and T represents a single bond) is preferred.

The resin (A) may have a repeating unit having an acid-decomposable group including an unsaturated bond as the repeating unit having an acid-decomposable group.

The repeating unit having an acid-decomposable group including an unsaturated bond is preferably a repeating unit represented by a formula (B).

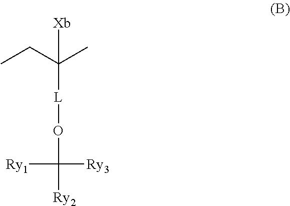

In the formula (B), Xb represents a hydrogen atom, a halogen atom, or an alkyl group that may have a substituent. L represents a single bond or a divalent linking group that may have a substituent. Ry₁ to Ry₃ each independently represent a linear or branched alkyl group, a monocyclic or polycyclic cycloalkyl group, an alkenyl group, an alkynyl group, or a monocyclic or polycyclic aryl group. Note that at least one of Ry₁ to Ry₃ represents an alkenyl group, an alkynyl group, a monocyclic or polycyclic cycloalkenyl group, or a monocyclic or polycyclic aryl group.

Two of Ry₁ to Ry₃ may be bonded together to form a monocyclic or polycyclic ring (such as a monocyclic or polycyclic cycloalkyl group or cycloalkenyl group).

The alkyl group that may have a substituent and is represented by Xb may be, for example, a methyl group or a group represented by —CH₂—R₁₁. R₁₁ represents a halogen atom (such as a fluorine atom), a hydroxy group, or a monovalent organic group, and examples thereof include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. Ru is preferably an alkyl group having 3 or less carbon atoms, more preferably a methyl group. Xb is preferably a hydrogen atom, a fluorine atom, a methyl group, a trifluoromethyl group, or a hydroxymethyl group.

The divalent linking group in L may be an -Rt- group, a —CO— group, a —COO-Rt- group, a —COO-Rt-CO— group, an -Rt-CO— group, or an —O-Rt- group. In the formulae, Rt represents an alkylene group, a cycloalkylene group, or an aromatic ring group and is preferably an aromatic ring group.

L is preferably an -Rt- group, a —CO— group, a —COO-Rt-CO— group, or an -Rt-CO— group. Rt may have a substituent such as a halogen atom, a hydroxy group, or an alkoxy group.

The alkyl group in Ry₁ to Ry₃ is preferably an alkyl group having 1 to 4 carbon atoms such as a methyl group, an ethyl group, a n-propyl group, an isopropyl group, a n-butyl group, an isobutyl group, or a t-butyl group.

The cycloalkyl group in Ry₁ to Ry₃ is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group.

The aryl group in Ry₁ to Ry₃ is preferably an aryl group having 6 to 10 carbon atoms and may be, for example, a phenyl group, a naphthyl group, or an anthryl group.

The alkenyl group in Ry₁ to Ry₃ is preferably a vinyl group.

The alkynyl group in Ry₁ to Ry₃ is preferably an ethynyl group.

The cycloalkenyl group in Ry₁ to Ry₃ preferably has a structure in which a monocyclic cycloalkyl group, such as a cyclopentyl group or a cyclohexyl group, partially includes a double bond.

The cycloalkyl group formed by bonding two of Ry₁ to Ry₃ together is preferably a monocyclic cycloalkyl group such as a cyclopentyl group or a cyclohexyl group, or a polycyclic cycloalkyl group such as a norbornyl group, a tetracyclodecanyl group, a tetracyclododecanyl group, or an adamantyl group. Of these, a monocyclic cycloalkyl group having 5 to 6 carbon atoms is more preferred.

In the cycloalkyl group or the cycloalkenyl group formed by bonding two of Ry₁ to Ry₃ together, for example, one of methylene groups forming the ring may be replaced by a heteroatom such as an oxygen atom, a group including a heteroatom, such as a carbonyl group, an —SO₂— group, or an —SO₃— group, a vinylidene group, or a combination thereof. In the cycloalkyl group or the cycloalkenyl group, one or more ethylene groups forming the cycloalkane ring or the cycloalkene ring may each be replaced by a vinylene group.

In a preferred embodiment of the repeating unit represented by the formula (B), for example, Ry₁ is a methyl group, an ethyl group, a vinyl group, an allyl group, or an aryl group, and Ry₂ and Ry₃ are bonded together to form the above-described cycloalkyl group or cycloalkenyl group.

When any of the above groups has a substituent, examples of the substituent include alkyl groups (having 1 to 4 carbon atoms), halogen atoms, a hydroxy group, alkoxy groups (having 1 to 4 carbon atoms), a carboxyl group, and alkoxycarbonyl groups (having 2 to 6 carbon atoms). The number of carbon atoms in the substituent is preferably 8 or less.

The repeating unit represented by the formula (B) is preferably an acid-decomposable (meth)acrylic acid tertiary ester-based repeating unit (a repeating unit in which Xb represents a hydrogen atom or a methyl group, and L represents a —CO— group), an acid-decomposable hydroxystyrene tertiary alkyl ether-based repeating unit (a repeating unit in which Xb represents a hydrogen atom or a methyl group, and L represents a phenylene group), or an acid-decomposable styrenecarboxylic acid tertiary ester-based repeating unit (a repeating unit in which Xb represents a hydrogen atom or a methyl group, and L represents an -Rt-CO— group (where Rt is an aromatic group)).

The content of the repeating unit having an acid-decomposable group including an unsaturated bond is preferably 15 mol % or more, more preferably 20 mol % or more, still more preferably 30 mol % or more relative to all the repeating units in the resin (A). The upper limit value of the content is preferably 80 mol % or less, more preferably 70 mol % or less, still more preferably 60 mol % or less relative to all the repeating units in the resin (A).

The content of the repeating unit having an acid-decomposable group is preferably 15 mol % or more, more preferably 20 mol % or more, still more preferably 30 mol % or more relative to all the repeating units in the resin (A). The upper limit value of the content is preferably 90 mol % or less, more preferably 80 mol % or less, still more preferably 70 mol % or less, particularly preferably 60 mol % or less relative to all the repeating units in the resin (A).

The resin (A) may include at least one repeating unit selected from the group consisting of Group A below and/or at least one repeating unit selected from the group consisting of Group B below.

Group A: Group consisting of the following repeating units (20) to (25)
(20) A repeating unit having an acid group, which will be described later
(21) A repeating unit having neither an acid-decomposable group nor an acid group but having a fluorine atom, a bromine atom, or an iodine atom, which will be described later
(22) A repeating unit having a lactone group, a sultone group, or a carbonate group, which will be described later
(23) A repeating unit having a photoacid generating group, which will be described later
(24) A repeating unit represented by a formula (V-1) or a formula (V-2) below, which will be described later
(25) A repeating unit for reducing the mobility of the main chain
Note that repeating units represented by formulae (A) to (C), which will be described later, each correspond to the (25) repeating unit for reducing the mobility of the main chain.

Group B: Group consisting of the following repeating units (30) to (32)
(30) A repeating unit having at least one group selected from the group consisting of lactone groups, sultone groups, carbonate groups, a hydroxy group, a cyano group, and alkali-soluble groups, which will be described later
(31) A repeating unit that has an alicyclic hydrocarbon structure and that does not exhibit acid decomposability, which will be described later
(32) A repeating unit having neither a hydroxy group nor a cyano group and represented by a formula (III), which will be described later

The resin (A) preferably has an acid group, and as described below, preferably includes a repeating unit having an acid group, and more preferably includes a repeating unit having a phenolic hydroxy group. The definition of the acid group will be described in a later part together with preferred embodiments of the repeating unit having an acid group. When the resin (A) has an acid group, a better interaction between the resin (A) and the acid generated from the photoacid generator is provided. As a result, the diffusion of the acid is further suppressed, and a pattern to be formed can have a more rectangular sectional shape.

The resin (A) may have at least one repeating unit selected from the group consisting of Group A described above. When the actinic ray-sensitive or radiation-sensitive resin composition is used as an actinic ray-sensitive or radiation-sensitive resin composition for EUV exposure, the resin (A) preferably has at least one repeating unit selected from the group consisting of Group A described above.

The resin (A) may include at least one of a fluorine atom or an iodine atom. When the actinic ray-sensitive or radiation-sensitive resin composition is used as an actinic ray-sensitive or radiation-sensitive resin composition for EUV exposure, the resin (A) preferably includes at least one of a fluorine atom or an iodine atom. When the resin (A) includes both a fluorine atom and an iodine atom, the resin (A) may have one type of repeating unit including both a fluorine atom and an iodine atom, or the resin (A) may include two types of repeating units including a repeating unit having a fluorine atom and a repeating unit including an iodine atom.

The resin (A) may have a repeating unit having an aromatic group. When the actinic ray-sensitive or radiation-sensitive resin composition is used as an actinic ray-sensitive or radiation-sensitive resin composition for EUV exposure, it is also preferable that the resin (A) have a repeating unit having an aromatic group.

The resin (A) may have at least one repeating unit selected from the group consisting of Group B described above. When the actinic ray-sensitive or radiation-sensitive resin composition is used as an actinic ray-sensitive or radiation-sensitive resin composition for ArF, the resin (A) preferably has at least one repeating unit selected from the group consisting of Group B described above.

When the actinic ray-sensitive or radiation-sensitive resin composition is used as an actinic ray-sensitive or radiation-sensitive resin composition for ArF, the resin (A) preferably includes neither a fluorine atom nor a silicon atom.

When the actinic ray-sensitive or radiation-sensitive resin composition is used as an actinic ray-sensitive or radiation-sensitive resin composition for ArF, the resin (A) preferably does not have an aromatic group.

### (Repeating Unit Having Acid Group)

The resin (A) may have a repeating unit having an acid group.

The acid group is preferably an acid group having a pKa of 13 or less. The acid dissociation constant of the acid group is preferably 13 or less, more preferably 3 to 13, still more preferably 5 to 10.

When the resin (A) has an acid group having a pKa of 13 or less, the content of the acid group in the resin (A) is not particularly limited but is often 0.2 to 6.0 mmol/g. In particular, the content of the acid group is preferably 0.8 to 6.0 mmol/g, more preferably 1.2 to 5.0 mmol/g, still more preferably 1.6 to 4.0 mmol/g. When the content of the acid group is within the above range, development proceeds satisfactorily, a pattern to be formed has a good shape, and thus high resolution is also achieved.

The acid group is preferably, for example, a carboxyl group, a phenolic hydroxy group, a fluorinated alcohol group (preferably a hexafluoroisopropanol group), a sulfonic group, a sulfonamide group, or an isopropanol group. Of these, a phenolic hydroxy group is more preferable.

In the hexafluoroisopropanol group, one or more (preferably one or two) of the fluorine atoms may each be substituted with a group other than a fluorine atom (such as an alkoxycarbonyl group). The acid group is also preferably —C(CF₃)(OH)—CF₂— formed in this manner. Alternatively, one or more of the fluorine atoms may each be substituted with a group other than a fluorine atom to form a ring including —C(CF₃)(OH)—CF₂—.

The repeating unit having an acid group is preferably a repeating unit different from the above-described repeating unit having a structure in which a polar group is protected with a group that leaves due to the action of an acid and a repeating unit having a lactone group, a sultone group, or a carbonate group described later.

The repeating unit having an acid group may have a fluorine atom or an iodine atom.

The repeating unit having an acid group is preferably a repeating unit represented by a formula (b1-1) below.

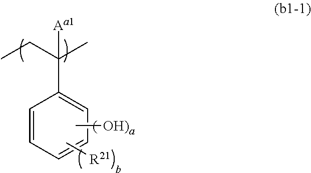

In the general formula (b1-1), A^{a1} represents a hydrogen atom, an alkyl group, a cycloalkyl group, a halogen atom, or a cyano group. R²¹ represents a halogen atom, an alkyl group, a cycloalkyl group, an aryl group, an alkenyl group, an aralkyl group, an alkoxy group, an alkylcarbonyloxy group, an alkylsulfonyloxy group, an alkyloxycarbonyl group, or an aryloxycarbonyl group. When a plurality of R²¹'s are present, the plurality of R²¹'s may be the same or different. When a plurality of R²¹'s are present, the plurality of R²¹'s may be bonded together to form a ring. R²¹ is preferably a hydrogen atom. a represents an integer of 1 to 3. b represents an integer of 0 to (5-a).

When the resin (A) includes a repeating unit having an acid group, the content of the repeating unit having an acid group is preferably 10 mol % or more, more preferably 15 mol % or more relative to all the repeating units in the resin (A). The upper limit value of the content is preferably 70 mol % or less, more preferably 65 mol % or less, still more preferably 60 mol % or less relative to all the repeating units in the resin (A).

### (Repeating Unit Having Neither Acid-Decomposable Group Nor Acid Group but Having Fluorine Atom, Bromine Atom, or Iodine Atom)

The resin (A) may have a repeating unit having neither an acid-decomposable group nor an acid group but having a fluorine atom, a bromine atom, or an iodine atom (hereinafter, also referred to as a unit X), in addition to the <Repeating unit having acid-decomposable group> and <Repeating unit having acid group> described above. The <Repeating unit having neither acid-decomposable group nor acid group but having fluorine atom, bromine atom, or iodine atom> referred to here is preferably different from other types of repeating units belonging to Group A, such as the <Repeating unit having lactone group, sultone group, or carbonate group> and the <Repeating unit having photoacid generating group>, which will be described later.

The unit X is preferably a repeating unit represented by a formula (C).

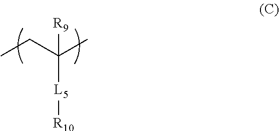

L₅ represents a single bond or an ester group. R₉ represents a hydrogen atom or an alkyl group that may have a fluorine atom or an iodine atom. R₁₀ represents a hydrogen atom, an alkyl group that may have a fluorine atom or an iodine atom, a cycloalkyl group that may have a fluorine atom or an iodine atom, an aryl group that may have a fluorine atom or an iodine atom, or a group provided by combining any of these groups.

The content of the unit X is preferably 0 mol % or more, more preferably 5 mol % or more, still more preferably 10 mol % or more relative to all the repeating units in the resin (A). The upper limit value of the content is preferably 50 mol % or less, more preferably 45 mol % or less, still more preferably 40 mol % or less relative to all the repeating units in the resin (A).

Among the repeating units of the resin (A), the total content of repeating units including at least one of a fluorine atom, a bromine atom, or an iodine atom is preferably 10 mol % or more, more preferably 20 mol % or more, still more preferably 30 mol % or more, particularly preferably 40 mol % or more relative to all the repeating units in the resin (A). The upper limit value is not particularly limited, but is, for example, 100 mol % or less relative to all the repeating units in the resin (A).

Examples of the repeating units including at least one of a fluorine atom, a bromine atom, or an iodine atom include a repeating unit having a fluorine atom, a bromine atom, or an iodine atom and having an acid-decomposable group, a repeating unit having a fluorine atom, a bromine atom, or an iodine atom and having an acid group, and a repeating unit having a fluorine atom, a bromine atom, or an iodine atom.

### (Repeating Unit Having Lactone Group, Sultone Group, or Carbonate Group)

The resin (A) may have a repeating unit having at least one selected from the group consisting of a lactone group, a sultone group, and a carbonate group (hereinafter, also referred to as a “unit Y”).

It is also preferable that the unit Y do not have an acid group such as a hydroxy group or a hexafluoropropanol group.

The lactone group or the sultone group at least has a lactone structure or a sultone structure. The lactone structure or the sultone structure is preferably a five- to seven-membered lactone structure or a five- to seven-membered sultone structure. In particular, a five- to seven-membered lactone structure to which another ring structure is fused so as to form a bicyclo structure or a spiro structure, or a five- to seven-membered sultone structure to which another ring structure is fused so as to form a bicyclo structure or a spiro structure is more preferred.

The resin (A) preferably has a repeating unit having a lactone group or a sultone group provided by abstracting one or more hydrogen atoms from ring member atoms of a lactone structure represented by any one of formulae (LC1-1) to (LC1-21) below or a sultone structure represented by any one of formulae (SL1-1) to (SL1-3) below, and the lactone group or the sultone group may be directly bonded to the main chain. For example, a ring member atom of the lactone group or the sultone group may constitute the main chain of the resin (A).

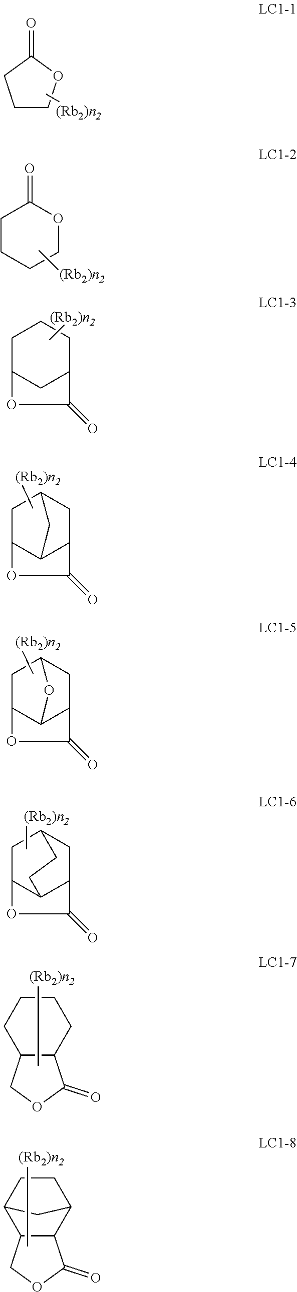

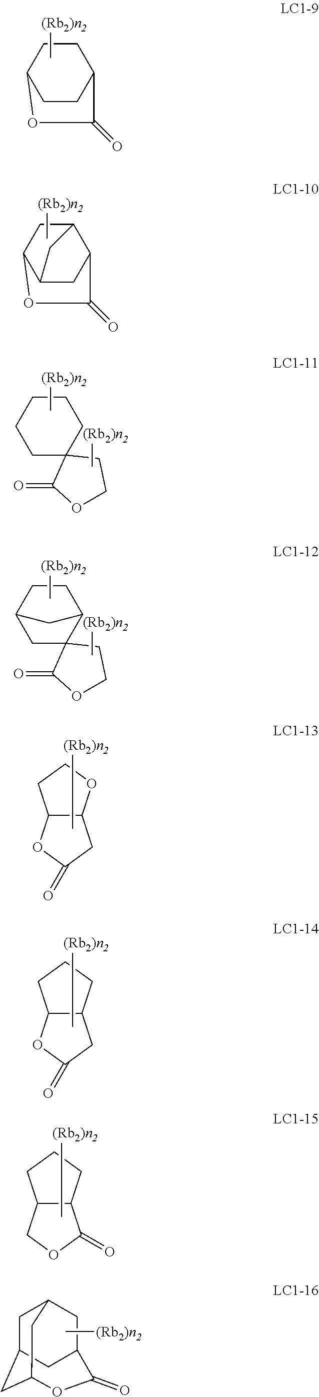

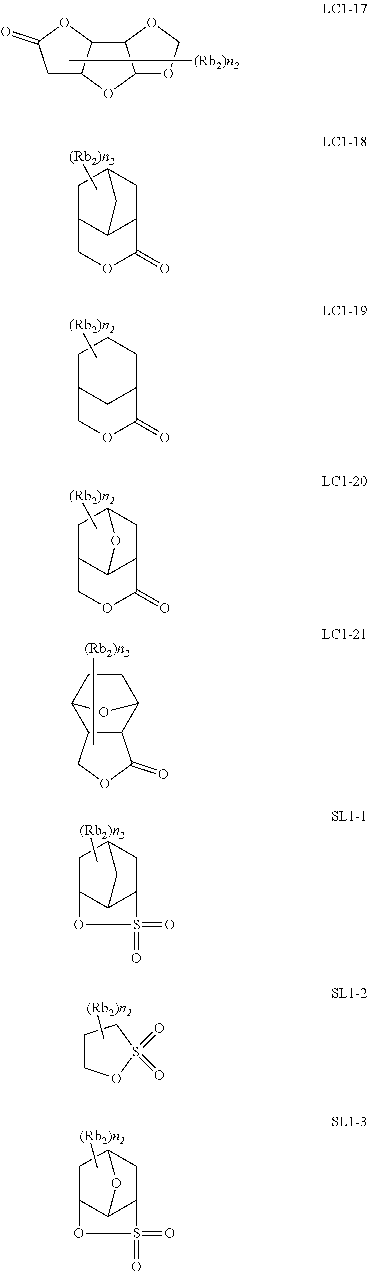

The lactone structure or the sultone structure may have a substituent (Rb₂). Preferred examples of the substituent (Rb₂) include alkyl groups having 1 to 8 carbon atoms, cycloalkyl groups having 4 to 7 carbon atoms, alkoxy groups having 1 to 8 carbon atoms, alkoxycarbonyl groups having 1 to 8 carbon atoms, a carboxyl group, halogen atoms, a cyano group, and acid-decomposable groups. n2 represents an integer of 0 to 4. When n2 is 2 or more, a plurality of Rb₂'s present may be different from each other, and the plurality of Rb₂'s present may be bonded together to form a ring.

The repeating unit having a group including the lactone structure represented by any one of the formulae (LC1-1) to (LC1-21) or the sultone structure represented by any one of the formulae (SL1-1) to (SL1-3) may be, for example, a repeating unit represented by a formula (AI) below.

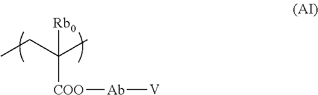

In the formula (AI), Rb₀ represents a hydrogen atom, a halogen atom, or an alkyl group having 1 to 4 carbon atoms. A preferred substituent that the alkyl group in Rb₀ may have may be a hydroxy group or a halogen atom.

Examples of the halogen atom in Rb₀ include a fluorine atom, a chlorine atom, a bromine atom, and an iodine atom. Rb₀ is preferably a hydrogen atom or a methyl group.

Ab represents a single bond, an alkylene group, a divalent linking group having a monocyclic or polycyclic alicyclic hydrocarbon structure, an ether group, an ester group, a carbonyl group, a carboxyl group, or a divalent linking group provided by combining any of these groups. Among these, Ab is preferably a single bond or a linking group represented by -Ab₁-CO₂—. Ab₁ is a linear or branched alkylene group or a monocyclic or polycyclic cycloalkylene group and is preferably a methylene group, an ethylene group, a cyclohexylene group, an adamantylene group, or a norbornylene group.

V represents a group provided by abstracting one hydrogen atom from a ring member atom of the lactone structure represented by any one of the formulae (LC1-1) to (LC1-21) or a group provided by abstracting one hydrogen atom from a ring member atom of the sultone structure represented by any one of the formulae (SL1-1) to (SL1-3).

When the repeating unit having a lactone group or a sultone group has an optical isomer, any optical isomer may be used. A single optical isomer may be used alone, or a mixture of a plurality of optical isomers may be used. When a single optical isomer is mainly used, its optical purity (ee) is preferably 90 or more, more preferably 95 or more.

The carbonate group is preferably a cyclic carbonate ester group.

The repeating unit having a cyclic carbonate ester group is preferably a repeating unit represented by a formula (A-1) below.

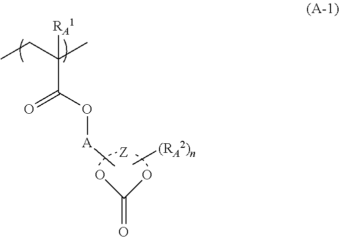

In the formula (A-1), R_{A}¹ represents a hydrogen atom, a halogen atom, or a monovalent organic group (preferably a methyl group). n represents an integer of 0 or more. R_{A}² represents a substituent. When n is 2 or more, a plurality of R_{A}²'s present may be the same or different from each other. A represents a single bond or a divalent linking group. The divalent linking group is preferably an alkylene group, a divalent linking group having a monocyclic or polycyclic alicyclic hydrocarbon structure, an ether group, an ester group, a carbonyl group, a carboxyl group, or a divalent linking group provided by combining any of these. Z represents an atomic group that forms a monocyclic ring or a polycyclic ring together with the group represented by —O—CO—O— in the formula.

When the resin (A) includes the unit Y, the content of the unit Y is preferably 1 mol % or more, more preferably 10 mol % or more relative to all the repeating units in the resin (A). The upper limit value of the content is preferably 85 mol % or less, more preferably 80 mol % or less, still more preferably 70 mol % or less, particularly preferably 60 mol % or less relative to all the repeating units in the resin (A).

### (Repeating Unit Having Photoacid Generating Group)

The resin (A) may have a repeating unit having a group that generates an acid upon irradiation with actinic rays or radiation (also referred to as a “photoacid generating group”) as a repeating unit other than the above repeating units.

The repeating unit having a photoacid generating group may be a repeating unit represented by a formula (4).

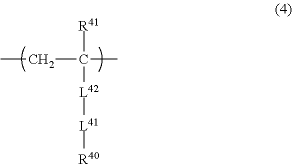

R⁴¹ represents a hydrogen atom or a methyl group. L⁴¹ represents a single bond or a divalent linking group. L⁴² represents a divalent linking group. R⁴⁰ represents a structural moiety that is decomposed by irradiation with actinic rays or radiation to generate an acid in the side chain.

Examples of the repeating unit represented by the formula (4) include the repeating units described in paragraphs [0094] to [0105] of JP2014-041327A and the repeating units described in paragraph [0094] of WO2018/193954A.

The content of the repeating unit having a photoacid generating group is preferably 1 mol % or more, more preferably 5 mol % or more relative to all the repeating units in the resin (A). The upper limit value of the content is preferably 40 mol % or less, more preferably 35 mol % or less, still more preferably 30 mol % or less relative to all the repeating units in the resin (A).

### (Repeating Unit Represented by Formula (V-1) or Formula (V-2) Below)

The resin (A) may have a repeating unit represented by a formula (V-1) below or a formula (V-2) below.

The repeating units represented by the formula (V-1) below and the formula (V-2) below are preferably repeating units different from the repeating units described above.

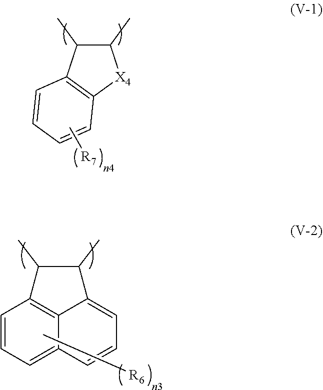

In the formulae,

R₆ and R₇ each independently represent a hydrogen atom, a hydroxy group, an alkyl group, an alkoxy group, an acyloxy group, a cyano group, a nitro group, an amino group, a halogen atom, an ester group (—OCOR or —COOR: R represents an alkyl group or fluorinated alkyl group having 1 to 6 carbon atoms), or a carboxyl group. The alkyl group is preferably a linear, branched, or cyclic alkyl group having 1 to 10 carbon atoms.
n3 represents an integer of 0 to 6.
n4 represents an integer of 0 to 4.
X₄ is a methylene group, an oxygen atom, or a sulfur atom.

Examples of the repeating unit represented by the formula (V-1) or (V-2) include the repeating units described in paragraph [0100] of WO2018/193954A.

The resin (A) can also include the “repeating unit for reducing the mobility of the main chain” described in paragraphs [0292] to [0308] of WO2023/106171A.

(Repeating Unit Having at Least One Group Selected from the Group Consisting of Lactone Groups, Sultone Groups, Carbonate Groups, Hydroxy Group, Cyano Group, and Alkali-Soluble Groups)

The resin (A) may have a repeating unit having at least one group selected from the group consisting of lactone groups, sultone groups, carbonate groups, a hydroxy group, a cyano group, and alkali-soluble groups.

Examples of the repeating unit that has a lactone group, a sultone group, or a carbonate group and that the resin (A) has include the repeating units described in the <Repeating unit having lactone group, sultone group, or carbonate group> above. The preferred content of the repeating unit is also as described in the <Repeating unit having lactone group, sultone group, or carbonate group> above.

The resin (A) may have a repeating unit having a hydroxy group or a cyano group. This results in improvement in adhesiveness to a substrate and affinity for a developer.

The repeating unit having a hydroxy group or a cyano group is preferably a repeating unit having an alicyclic hydrocarbon structure substituted with a hydroxy group or a cyano group.

Preferably, the repeating unit having a hydroxy group or a cyano group has no acid-decomposable group. Examples of the repeating unit having a hydroxy group or a cyano group include those described in paragraphs [0081] to [0084] of JP2014-098921A.

The resin (A) may have a repeating unit having an alkali-soluble group.

Examples of the alkali-soluble group include a carboxyl group, a sulfonamide group, a sulfonylimide group, a bissulfonylimide group, and aliphatic alcohol groups substituted with an electron-withdrawing group at the α-position (for example, a hexafluoroisopropanol group), and the alkali-soluble group is preferably a carboxyl group. When the resin (A) includes a repeating unit having an alkali-soluble group, the resolution is increased in contact hole applications. Examples of the repeating unit having an alkali-soluble group include those described in paragraphs [0085] and [0086] of JP2014-098921A.

(Repeating Unit that has Alicyclic Hydrocarbon Structure and that does not Exhibit Acid Decomposability)

The resin (A) may have a repeating unit that has an alicyclic hydrocarbon structure and that does not exhibit acid decomposability. This makes it possible to reduce leaching of low-molecular-weight components, during liquid immersion exposure, from the resist film into the immersion liquid. The repeating unit that has an alicyclic hydrocarbon structure and that does not exhibit acid decomposability may be, for example, a repeating unit derived from 1-adamantyl (meth)acrylate, diamantyl (meth)acrylate, tricyclodecanyl (meth)acrylate, or cyclohexyl (meth)acrylate.

### (Repeating Unit Having Neither Hydroxy Group Nor Cyano Group and Represented by Formula (III))

The resin (A) may have a repeating unit having neither a hydroxy group nor a cyano group and represented by a formula (III).

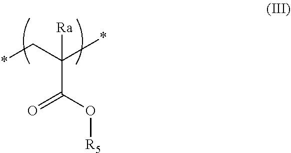

In the formula (III), R₅ represents a hydrocarbon group having at least one ring structure and having neither a hydroxy group nor a cyano group.

Ra represents a hydrogen atom, an alkyl group, or a —CH₂—O—Ra₂ group. In the formula, Ra₂ represents a hydrogen atom, an alkyl group, or an acyl group.

Examples of the repeating unit having neither a hydroxy group nor a cyano group and represented by the formula (III) include those described in paragraphs [0087] to [0094] of JP2014-098921A.

### (Other Repeating Units)

Furthermore, the resin (A) may have another repeating unit other than the above-described repeating units.

For example, the resin (A) may have a repeating unit selected from the group consisting of a repeating unit having an oxathiane ring group, a repeating unit having an oxazolone ring group, a repeating unit having a dioxane ring group, and a repeating unit having a hydantoin ring group.

The resin (A) may have, in addition to the above-described repeating units, various repeating units for the purpose of adjusting dry etching resistance, suitability for a standard developer, adhesiveness to a substrate, resist profile, resolution, heat resistance, sensitivity, and the like.

For the resin (A), particularly in the case where the composition of the present invention is used as an actinic ray-sensitive or radiation-sensitive resin composition for ArF, all the repeating units are preferably constituted by repeating units derived from compounds having ethylenically unsaturated bonds. In particular, all the repeating units are also preferably constituted by (meth)acrylate-based repeating units. When all the repeating units are constituted by (meth)acrylate-based repeating units, all the repeating units may be methacrylate-based repeating units, all the repeating units may be acrylate-based repeating units, or all the repeating units may each be a methacrylate-based repeating unit or an acrylate-based repeating unit. The acrylate-based repeating units preferably account for 50 mol % or less of all the repeating units.

The resin (A) can be synthesized by an ordinary method (for example, radical polymerization).

The resin (A) preferably has a weight-average molecular weight (Mw) of 30,000 or less, more preferably 1,000 to 30,000, still more preferably 3,000 to 30,000, particularly preferably 5,000 to 15,000 in terms of polystyrene as determined by the GPC method.

The dispersity (molecular weight distribution, Mw/Mn) of the resin (A) is preferably 1 to 5, more preferably 1 to 3, still more preferably 1.2 to 3.0, particularly preferably 1.2 to 2.0. The smaller the dispersity, the better the resolution and the resist profile, the smoother the sidewalls of the resist pattern, and the better the roughness performance.

In the actinic ray-sensitive or radiation-sensitive resin composition, the content of the resin (A) is preferably 30.0 to 99.9 mass %, more preferably 40.0 to 99.9 mass %, still more preferably 60.0 to 90.0 mass % relative to the total solid contents of the actinic ray-sensitive or radiation-sensitive resin composition.

Such resins (A) may be used alone or in combination of two or more thereof.

[Compound (C) that Generates Acid Upon Irradiation with Actinic Rays or Radiation]

The composition of the present invention may further include a compound (C) (hereinafter, also referred to as “photoacid generator (C)”) which is a compound that generates an acid upon irradiation with actinic rays or radiation and is different from the compound (B).

The compound (C) may be in the form of a low-molecular-weight compound, or may be in the form of being incorporated into a part of a polymer. Alternatively, the form of a low-molecular-weight compound and the form of being incorporated into a part of a polymer may be used in combination.

When the compound (C) is in the form of a low-molecular-weight compound, the molecular weight of the compound (C) is preferably 5,000 or less, more preferably 4,000 or less, still more preferably 3,000 or less. The lower limit is not particularly limited, but is preferably 100 or more.

When the compound (C) is in the form of being incorporated into a part of a polymer, the compound (C) may be incorporated into a part of the resin (A) or incorporated into a resin different from the resin (A).

The compound (C) is preferably in the form of a low-molecular-weight compound.

The compound (C) is, for example, a compound (onium salt) represented by “M⁺X⁻ and is preferably a compound that generates an organic acid upon exposure.

Examples of the organic acid include sulfonic acids (such as aliphatic sulfonic acids, aromatic sulfonic acids, and camphorsulfonic acid), carboxylic acids (such as aliphatic carboxylic acids, aromatic carboxylic acids, and aralkyl carboxylic acids), carbonylsulfonylimidic acid, bis(alkylsulfonyl)imidic acids, and tris(alkylsulfonyl)methide acids.

In the compound represented by “M⁺X⁻”, M⁺ represents a cation, and preferably represents an organic cation.

Specific examples and preferred ranges of M⁺ are the same as the specific examples and preferred ranges of M₁⁺ and M₂⁺ in the compound (B) described above.

In the compound represented by “M⁺ X⁻”, X⁻ represents an anion, and preferably represents an organic anion.

The organic anion is not particularly limited, and may be a mono-, di-, or higher valent organic anion.

The organic anion is preferably an anion having an extremely low ability to cause a nucleophilic reaction, more preferably a non-nucleophilic anion.

Examples of the non-nucleophilic anion include sulfonate anions (such as aliphatic sulfonate anions, aromatic sulfonate anions, and a camphorsulfonate anion), carboxylate anions (such as aliphatic carboxylate anions, aromatic carboxylate anions, and aralkyl carboxylate anions), sulfonylimide anions, bis(alkylsulfonyl)imide anions, and tris(alkylsulfonyl)methide anions.

The aliphatic moieties in the aliphatic sulfonate anions and the aliphatic carboxylate anions may each be a linear or branched alkyl group or a cycloalkyl group, and are each preferably a linear or branched alkyl group having 1 to 30 carbon atoms, or a cycloalkyl group having 3 to 30 carbon atoms.

The above alkyl group may be, for example, a fluoroalkyl group (that may have a substituent other than a fluorine atom or may be a perfluoroalkyl group).

The aryl groups in the aromatic sulfonate anions and the aromatic carboxylate anions are each preferably an aryl group having 6 to 14 carbon atoms, and examples thereof include a phenyl group, a tolyl group, and a naphthyl group.

The above-mentioned alkyl group, cycloalkyl group, and aryl group may have a substituent. Examples of the substituent include, but are not particularly limited to, a nitro group, halogen atoms such as a fluorine atom and a chlorine atom, a carboxyl group, a hydroxy group, an amino group, a cyano group, alkoxy groups (preferably having 1 to 15 carbon atoms), alkyl groups (preferably having 1 to 10 carbon atoms), cycloalkyl groups (preferably having 3 to 15 carbon atoms), aryl groups (preferably having 6 to 14 carbon atoms), alkoxycarbonyl groups (preferably having 2 to 7 carbon atoms), acyl groups (preferably having 2 to 12 carbon atoms), alkoxycarbonyloxy groups (preferably having 2 to 7 carbon atoms), alkylthio groups (preferably having 1 to 15 carbon atoms), alkylsulfonyl groups (preferably having 1 to 15 carbon atoms), alkyliminosulfonyl groups (preferably having 1 to 15 carbon atoms), and aryloxysulfonyl groups (preferably having 6 to 20 carbon atoms).

The aralkyl groups in the aralkyl carboxylate anions are each preferably an aralkyl group having 7 to 14 carbon atoms.

Examples of the aralkyl group having 7 to 14 carbon atoms include a benzyl group, a phenethyl group, a naphthylmethyl group, a naphthylethyl group, and a naphthylbutyl group.

An example of the sulfonylimide anions is a saccharin anion.

The alkyl groups in the bis(alkylsulfonyl)imide anions and the tris(alkylsulfonyl)methide anions are preferably alkyl groups having 1 to 5 carbon atoms. A substituent of such an alkyl group may be a halogen atom, an alkyl group substituted with a halogen atom, an alkoxy group, an alkylthio group, an alkyloxysulfonyl group, an aryloxysulfonyl group, or a cycloalkylaryloxysulfonyl group, and is preferably a fluorine atom or an alkyl group substituted with a fluorine atom.

The alkyl groups in such a bis(alkylsulfonyl)imide anion may be bonded together to form a ring structure. This increases the acid strength.

Other examples of the non-nucleophilic anion include fluorinated phosphorus (for example, PF₆⁻), fluorinated boron (for example, BF₄⁻), and fluorinated antimony (for example, SbF₆⁻).

As the non-nucleophilic anion, an anion represented by a formula (AN1) below is also preferred.

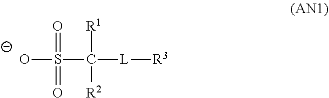

In the formula (AN1), R¹ and R² each independently represent a hydrogen atom or a substituent.

The substituent is not particularly limited, but is preferably a group that is not an electron-withdrawing group. Examples of the group that is not an electron-withdrawing group include hydrocarbon groups, a hydroxy group, oxyhydrocarbon groups, oxycarbonyl hydrocarbon groups, an amino group, hydrocarbon-substituted amino groups, and hydrocarbon-substituted amide groups.

Each group that is not an electron-withdrawing group is preferably independently —R′, —OH, —OR′, —OCOR′, —NH₂, —NR′₂, —NHR′, or —NHCOR′. R′ is a monovalent hydrocarbon group.

Examples of the monovalent hydrocarbon group represented by R′ include monovalent linear or branched hydrocarbon groups such as alkyl groups, e.g., a methyl group, an ethyl group, a propyl group, and a butyl group, alkenyl groups, e.g., an ethenyl group, a propenyl group, and a butenyl group, and alkynyl groups, e.g., an ethynyl group, a propynyl group, and a butynyl group; monovalent alicyclic hydrocarbon groups such as cycloalkyl groups, e.g., a cyclopropyl group, a cyclobutyl group, a cyclopentyl group, a cyclohexyl group, a norbornyl group, and an adamantyl group, and cycloalkenyl groups, e.g., a cyclopropenyl group, a cyclobutenyl group, a cyclopentenyl group, and a norbornenyl group; and monovalent aromatic hydrocarbon groups such as aryl groups, e.g., a phenyl group, a tolyl group, a xylyl group, a mesityl group, a naphthyl group, a methylnaphthyl group, an anthryl group, and a methylanthryl group, and aralkyl groups, e.g., a benzyl group, a phenethyl group, a phenylpropyl group, a naphthylmethyl group, and an anthrylmethyl group.

Of these, R¹ and R² are each independently preferably a hydrocarbon group (preferably a cycloalkyl group) or a hydrogen atom.

L represents a divalent linking group.

When a plurality of L's are present, L's may be the same or different.

Examples of the divalent linking group include —O—CO—O—, —COO—, —CONH—, —CO—, —O—, —S—, —SO—, —SO₂—, alkylene groups (preferably having 1 to 6 carbon atoms), cycloalkylene groups (preferably having 3 to 15 carbon atoms), alkenylene groups (preferably having 2 to 6 carbon atoms), and divalent linking groups provided by combining a plurality of these groups. Among these, the divalent linking group is preferably —O—CO—O—, —COO—, —CONH—, —CO—, —O—, —SO₂—, —O—CO—O-alkylene group-, —COO-alkylene group-, or —CONH-alkylene group-, more preferably —O—CO—O—, —O—CO—O-alkylene group-, —COO—, —CONH—, —SO₂—, or —COO-alkylene group-.

L is preferably, for example, a group represented by a formula (AN1-1) below.

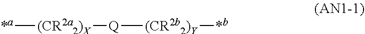

In the formula (AN1-1), *^{a} represents a bonding site to R³ in the formula (AN1).

*^{b} represents a bonding site to —C(R¹)(R²)— in the formula (AN1).

X and Y each independently represent an integer of 0 to 10 and preferably an integer of 0 to 3.

R^{2a} and R^{2b} each independently represent a hydrogen atom or a substituent.

When a plurality of R^{2a}'s and a plurality of R^{2b}'s are present, the plurality of R^{2a}'s present may be the same or different and the plurality of R^{2b}'s present may be the same or different.

However, when Y is 1 or more, R^{2b}'s in CR^{2b}₂ directly bonded to —C(R¹)(R²)— in the formula (AN1) are not fluorine atoms.

Q represents *^{A}—O—CO—O—*^{B}, *^{A}—CO—*^{B}, *^{A}—CO—O—*^{B}, *^{A}—O—CO—*^{B}, *^{A}—O—*^{B}, *^{A}—S—*^{B}, or *^{A}—SO₂—*^{B}.

However, when X+Y in the formula (AN1-1) is 1 or more and R^{2a}'s and R^{2b}'s in the formula (AN1-1) are all hydrogen atoms, Q represents *^{A}—O—CO—O—*^{B}, *^{A}—CO—*^{B}, *^{A}—O—CO—*^{B}, *^{A}—O—*^{B}, *^{A}—S—*^{B}, or *^{A}—SO₂—*^{B}.

*^{A} represents a bonding site on the R³ side in the formula (AN1), and *^{B} represents a bonding site on the —SO₃⁻ side in the formula (AN1).

In the formula (AN1), R³ represents an organic group.

The organic group is not particularly limited as long as it has 1 or more carbon atoms, and may be a linear group (for example, a linear alkyl group), a branched group (for example, a branched alkyl group such as a t-butyl group), or a cyclic group. The organic group may have or may not have a substituent. The organic group may have or may not have a heteroatom (such as an oxygen atom, a sulfur atom, and/or a nitrogen atom).

In particular, R³ is preferably an organic group having a ring structure. The ring structure may be monocyclic or polycyclic, and may have a substituent. The ring in the organic group including a ring structure is preferably directly bonded to L in the formula (AN1).

The organic group having a ring structure may have or may not have, for example, a heteroatom (such as an oxygen atom, a sulfur atom, and/or a nitrogen atom). One or more carbon atoms forming the ring structure may each be substituted with a heteroatom.

The organic group having a ring structure is preferably, for example, a hydrocarbon group having a ring structure, a lactone ring group, or a sultone ring group. In particular, the organic group having a ring structure is preferably a hydrocarbon group having a ring structure.

The hydrocarbon group having a ring structure is preferably a monocyclic or polycyclic cycloalkyl group. These groups may have a substituent.

The cycloalkyl group may be monocyclic (such as a cyclohexyl group) or polycyclic (such as an adamantyl group), and preferably has 5 to 12 carbon atoms.

The lactone group and sultone group are preferably, for example, groups formed by removing one hydrogen atom from a ring member atom constituting the lactone structure or the sultone structure in any of the structures represented by the formulae (LC1-1) to (LC1-21) described above and the structures represented by the formulae (SL1-1) to (SL1-3) described above.

The non-nucleophilic anion may be a benzenesulfonate anion, and is preferably a benzenesulfonate anion substituted with a branched alkyl group or a cycloalkyl group.

As the non-nucleophilic anion, an anion represented by a formula (AN2) below is also preferred.

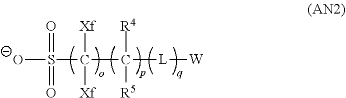

In the formula (AN2), o represents an integer of 1 to 3. p represents an integer of 0 to 10. q represents an integer of 0 to 10.

Xf's each represent a hydrogen atom, a fluorine atom, an alkyl group substituted with at least one fluorine atom, or an organic group having no fluorine atom. The number of carbon atoms of the alkyl group is preferably 1 to 10, more preferably 1 to 4. The alkyl group substituted with at least one fluorine atom is preferably a perfluoroalkyl group.

Xf's are each preferably a fluorine atom or a perfluoroalkyl group having 1 to 4 carbon atoms, more preferably a fluorine atom or CF₃. Still more preferably, both Xf's are fluorine atoms.

R⁴ and R⁵ each independently represent a hydrogen atom, a fluorine atom, an alkyl group, or an alkyl group substituted with at least one fluorine atom. When a plurality of R⁴'s and a plurality of R⁵'s are present, the plurality of R⁴'s may be the same or different and the plurality of R⁵'s may be the same or different.

The alkyl group represented by R⁴ and R⁵ preferably has 1 to 4 carbon atoms. The alkyl group may have a substituent. R⁴ and R⁵ are preferably hydrogen atoms.

L represents a divalent linking group. The definition of Lis the same as that of L in the formula (AN1).

When a plurality of L's are present, L's may be the same or different.

W represents an organic group including a ring structure. In particular, a cyclic organic group is preferable.

The cyclic organic group may be, for example, an alicyclic group, an aryl group, or a heterocyclic group.

The alicyclic group may be monocyclic or polycyclic. Examples of the monocyclic alicyclic group include monocyclic cycloalkyl groups such as a cyclopentyl group, a cyclohexyl group, and a cyclooctyl group. Examples of the polycyclic alicyclic group include polycyclic cycloalkyl groups such as a norbornyl group, a tricyclodecanyl group, a tetracyclodecanyl group, a tetracyclododecanyl group, and an adamantyl group. Of these, alicyclic groups having a bulky structure with 7 or more carbon atoms, such as a norbornyl group, a tricyclodecanyl group, a tetracyclodecanyl group, a tetracyclododecanyl group, and an adamantyl group, are preferred.

The aryl group may be monocyclic or polycyclic. Examples of the aryl group include a phenyl group, a naphthyl group, a phenanthryl group, and an anthryl group.

The heterocyclic group may be monocyclic or polycyclic. In particular, in the case of a polycyclic heterocyclic group, diffusion of the acid can be further suppressed. The heterocyclic group may have aromaticity or may not have aromaticity. Examples of the heterocycle having aromaticity include a furan ring, a thiophene ring, a benzofuran ring, a benzothiophene ring, a dibenzofuran ring, a dibenzothiophene ring, and a pyridine ring. Examples of the heterocycle having no aromaticity include a tetrahydropyran ring, a lactone ring, a sultone ring, and a decahydroisoquinoline ring. The heterocycle in the heterocyclic group is preferably a furan ring, a thiophene ring, a pyridine ring, or a decahydroisoquinoline ring.

The cyclic organic group may have a substituent. Examples of the substituent include alkyl groups (which may be linear or branched and preferably have 1 to 12 carbon atoms), cycloalkyl groups (which may be monocyclic, polycyclic, or spirocyclic and preferably have 3 to 20 carbon atoms), aryl groups (preferably having 6 to 14 carbon atoms), a hydroxy group, alkoxy groups, an ester group, an amide group, a urethane group, a ureido group, a thioether group, a sulfonamide group, and a sulfonic acid ester group. The carbon constituting the cyclic organic group (carbon that contributes to ring formation) may be a carbonyl carbon.

The anion represented by the formula (AN2) is preferably SO₃⁻—CF₂—CH₂—OCO-(L)_{q′}-W, SO₃⁻—CF₂—CHF—CH₂—OCO-(L)_{q′}-W, SO₃⁻—CF₂—COO-(L)_{q′}-W, SO₃⁻—CF₂—CF₂—CH₂—CH₂-(L)_{q}-W, or SO₃⁻—CF₂—CH(CF₃)—OCO-(L)_{q′},-W. Here, L, q, and W are the same as those in the formula (AN2). q′ represents an integer of 0 to 10.

As the non-nucleophilic anion, an aromatic sulfonate anion represented by a formula (AN3) below is also preferred.

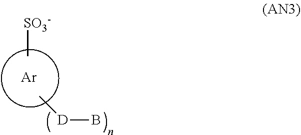

In the formula (AN3), Ar represents an aryl group (such as a phenyl group) and may further have a substituent other than the sulfonate anion and the -(D-B) group. Examples of the substituent that Ar may further have include a fluorine atom and a hydroxy group. n represents an integer of 0 or more. n is preferably 1 to 4, more preferably 2 to 3, still more preferably 3.

D represents a single bond or a divalent linking group. Examples of the divalent linking group include an ether group, a thioether group, a carbonyl group, a sulfoxide group, a sulfone group, a sulfonic acid ester group, an ester group, and groups provided by combining two or more of these groups.

B represents a hydrocarbon group.

B is preferably an aliphatic hydrocarbon group, more preferably an isopropyl group, a cyclohexyl group, or an aryl group that may further have a substituent (such as a tricyclohexylphenyl group).

The non-nucleophilic anion is also preferably a disulfonamide anion.

The disulfonamide anion is, for example, an anion represented by N⁻(SO₂—R^{q})₂.

Here, each R^{q} represents an alkyl group that may have a substituent and is preferably a fluoroalkyl group, more preferably a perfluoroalkyl group. Two R^{q}'s may be bonded together to form a ring. The group formed by bonding two R^{q}'s together is preferably an alkylene group that may have a substituent, more preferably a fluoroalkylene group, still more preferably a perfluoroalkylene group. The alkylene group preferably has 2 to 4 carbon atoms.

The compound (C) is also preferably at least one selected from the group consisting of compounds (I) and (II).

### (Compound (I))

The compound (I) is a compound that has one or more structural moieties X_{C} described below and one or more structural moieties Y_{C} described below and that generates an acid including a first acidic moiety described below and derived from the structural moiety X_{C} and a second acidic moiety described below and derived from the structural moiety Y_{C} upon irradiation with actinic rays or radiation.

Structural moiety X_{C}: A structural moiety that is constituted by an anionic moiety A_{1C}⁻ and a cationic moiety M_{1C}⁺ and that forms the first acidic moiety represented by HA_{1C} upon irradiation with actinic rays or radiation

Structural moiety Y_{C}: A structural moiety that is constituted by an anionic moiety A_{2C}⁻ and a cationic moiety M_{2C}⁺ and that forms the second acidic moiety represented by HA_{2C} upon irradiation with actinic rays or radiation

The compound (I) satisfies the following condition I_{C}. However, the compound (I) is a compound other than the compound (B) described above.

Condition I_{C}: A compound PI_{C} obtained by replacing the cationic moiety M_{1C}⁺ in the structural moiety X_{C} and the cationic moiety M_{2C}⁺ in the structural moiety Y_{C} in the compound (I) with H⁺ has an acid dissociation constant a1_{C} derived from an acidic moiety represented by HA_{1C} obtained by replacing the cationic moiety M_{1C}⁺ in the structural moiety X_{C} with H⁺ and an acid dissociation constant a2_{C} derived from an acidic moiety represented by HA_{2C} obtained by replacing the cationic moiety M_{2C}⁺ in the structural moiety Y_{C} with H⁺, and the acid dissociation constant a2_{C} is larger than the acid dissociation constant alc.

The condition I_{C} will be more specifically described below.

When the compound (I) is, for example, a compound that generates an acid having one first acidic moiety derived from the structural moiety X_{C} and one second acidic moiety derived from the structural moiety Y_{C}, the compound PI_{C} corresponds to a “compound having HA_{1C} and HA_{2C}”.

The acid dissociation constant a1_{C} and the acid dissociation constant a2_{C} of the compound PI_{C} are more specifically described as follows. In determination of the acid dissociation constants of the compound PI_{C}, the pKa determined when the compound PI_{C} turns into a “compound having A_{1C} and HA_{2C}” is the acid dissociation constant a1_{C}, and the pKa determined when the “compound having A_{1c}⁻ and HA_{2C}” turns into a “compound having A_{1C}⁻ and A_{2C}⁻” is the acid dissociation constant a2_{C}.

When the compound (I) is, for example, a compound that generates an acid having two first acidic moieties derived from the structural moiety X_{C} and one second acidic moiety derived from the structural moiety Y_{C}, the compound PI_{C} corresponds to a “compound having two HA_{1C}'s and one HA_{2C}”.

In determination of the acid dissociation constants of the compound PI_{C}, the acid dissociation constant determined when the compound PI_{C} turns into a “compound having one A_{1C}⁻, one HA_{1C}, and one HA_{2C}” and the acid dissociation constant determined when the “compound having one A_{1C}⁻, one HA_{1C}, and one HA_{2C}” turns into a “compound having two A_{1C}⁻'s and one HA_{2C}” each correspond to the above-described acid dissociation constant a1_{C}. The acid dissociation constant determined when the “compound having two A_{1C}⁻'s and one HA_{2C}” turns into a “compound having two A_{1C}⁻'s and A_{2C}⁻” corresponds to the acid dissociation constant a2_{C}. That is, when the compound PI_{C} has a plurality of acid dissociation constants derived from acidic moieties represented by HA_{1C} obtained by replacing the cationic moiety M_{1C}⁺ in the structural moiety X_{C} with H⁺, the value of the acid dissociation constant a2_{C} is larger than the largest value among the plurality of acid dissociation constants a1_{C}. Note that, when the acid dissociation constant determined when the compound PI_{C} turns into a “compound having one A_{1C}⁻, one HA_{1C}, and one HA_{2C}” is defined as aa_{C} and the acid dissociation constant determined when the “compound having one A_{1C}⁻, one HA_{1C}, and one HA_{2C}” turns into a “compound having two A_{1C}⁻'s and one HA_{2C}” is defined as ab_{C}, the relation between aa_{C} and ab_{C} satisfies aa_{C}<ab_{C}.

The acid dissociation constant a1_{C} and the acid dissociation constant a2_{C} can be determined by the above-described method of measuring an acid dissociation constant.

The compound PI_{C} corresponds to an acid generated when the compound (I) is irradiated with actinic rays or radiation.

When the compound (I) has two or more structural moieties X_{C}, the structural moieties X_{C} may be the same or different. Two or more A_{1C}⁻'s and two or more M_{1C}⁺'s may be individually the same or different.

In the compound (I), A_{1C}⁻ and A_{2C}⁻, and M_{1C}⁺ and M_{2C}⁺ may be individually the same or different, but A_{1C}⁻ and A_{2C}⁻ are preferably different.

In the compound PI_{C}, the difference (absolute value) between the acid dissociation constant a1_{C} (in the case where a plurality of acid dissociation constants a1_{C} are present, the maximum value thereof) and the acid dissociation constant a2_{C} is preferably 0.1 or more, more preferably 0.5 or more, still more preferably 1.0 or more. The upper limit value of the difference (absolute value) between the acid dissociation constant a1_{C} (in the case where a plurality of acid dissociation constants a1_{C} are present, the maximum value thereof) and the acid dissociation constant a2_{C} is not particularly limited, but is, for example, 16 or less.

In the compound PI_{C}, the acid dissociation constant a2_{C} is preferably 20 or less, more preferably 15 or less. The lower limit value of the acid dissociation constant a2_{C} is preferably −4.0 or more.

In the compound PI_{C}, the acid dissociation constant a1_{C} is preferably 2.0 or less, more preferably 0 or less. The lower limit value of the acid dissociation constant a1_{C} is preferably −20.0 or more.

The anionic moiety A_{1C}⁻ and the anionic moiety A_{2C}⁻ are structural moieties including a negatively charged atom or atomic group and are, for example, structural moieties selected from the group consisting of formulae (AA-1) to (AA-3) and formulae (BB-1) to (BB-6) below.

The anionic moiety A_{1C}⁻ is preferably one that can form an acidic moiety having a small acid dissociation constant, in particular, more preferably any one of the formulae (AA-1) to (AA-3), still more preferably any one of the formulae (AA-1) and (AA-3).

The anionic moiety A_{2C}⁻ is preferably one that can form an acidic moiety having a larger acid dissociation constant than the anionic moiety A_{1C}⁻, more preferably any one of the formulae (BB-1) to (BB-6), still more preferably any one of the formulae (BB-1) and (BB-4).

In the formulae (AA-1) to (AA-3) and the formulae (BB-1) to (BB-6) below, * represents a bonding site.

In the formula (AA-2), each R^{A} represents a monovalent organic group. The monovalent organic group represented by R^{A} is not particularly limited and may be, for example, a cyano group, a trifluoromethyl group, or a methanesulfonyl group.

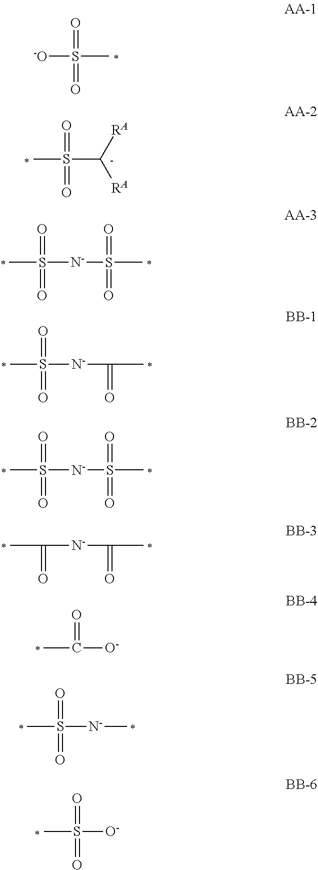

The cationic moiety M_{1C}⁺ and the cationic moiety M_{2C}⁺ are structural moieties including a positively charged atom or atomic group and are, for example, singly charged organic cations. Examples of organic cations include the organic cations represented by M₁⁺ and M₂⁺ in the compound (B) described above.

### (Compound (II))

The compound (II) is a compound that has two or more structural moieties X_{C} described above and one or more structural moieties Z_{C} described below and that generates an acid including two or more first acidic moieties derived from the structural moieties X_{C} and the structural moiety Z_{C} upon irradiation with actinic rays or radiation.

### Structural Moiety Z_{C}: A Nonionic Moiety Capable of Neutralizing an Acid

In the compound (II), the definition of the structural moiety X_{C}, and the definitions of A_{1C}⁻ and M_{1C}⁺ are the same as the definition of the structural moiety X_{C}, and the definitions of A_{1C}⁻ and M_{1C}⁺, respectively, in the compound (I) described above, and preferred embodiments are also the same as those of the structural moiety X_{C}, A_{1C}⁻, and M_{1C}⁺ in the compound (I).

In a compound PIIc obtained by replacing each of the cationic moieties M_{1C}⁺ in the structural moieties X_{C} in the compound (II) with H⁺, a preferred range of the acid dissociation constant a1_{C} derived from the acidic moiety represented by HA_{1C} obtained by replacing the cationic moiety M_{1C}⁺ in one of the structural moieties X_{C} by H⁺ is the same as that of the acid dissociation constant a1_{C} in the compound PI_{C}.

When the compound (II) is, for example, a compound that generates an acid having two first acidic moieties derived from the structural moieties X_{C} and the structural moiety Z_{C}, the compound PIIc corresponds to a “compound having two HA_{1C}'s”. In determination of the acid dissociation constants of this compound PIIc, the acid dissociation constant determined when the compound PIIc turns into a “compound having one A_{1C}⁻ and one HA_{1C}” and the acid dissociation constant determined when the “compound having one A_{1C}⁻ and one HA_{1C}” turns into a “compound having two A_{1C}⁻'s” each correspond to the acid dissociation constant a1_{C}.

The acid dissociation constant a1_{C} can be determined by the above-described method of measuring an acid dissociation constant.

The compound PIIc corresponds to an acid generated when the compound (II) is irradiated with actinic rays or radiation.

The two or more structural moieties X_{C} may be the same or different. Two or more A_{1C}⁻'s and two or more M_{1C}⁺'s may be individually the same or different.

The nonionic moiety capable of neutralizing an acid in the structural moiety Z_{C} is not particularly limited, and is preferably, for example, a moiety including a functional group having an electron or a group that can electrostatically interact with a proton.

Examples of the functional group having an electron or a group that can electrostatically interact with a proton include a functional group having a macrocyclic structure such as a cyclic polyether, and a functional group having a nitrogen atom having an unshared electron pair that does not contribute to π-conjugation. The nitrogen atom having an unshared electron pair that does not contribute to π-conjugation is, for example, a nitrogen atom having a partial structure represented by any of the following formulae.

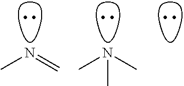

### Unshared Electron Pair

Examples of the partial structure of the functional group having an electron or a group that can electrostatically interact with a proton include crown ether structures, azacrown ether structures, primary to tertiary amine structures, a pyridine structure, an imidazole structure, and a pyrazine structure. Among these, primary to tertiary amine structures are preferable.

Examples of moieties that are not cations and that the compound (I) and the compound (II) may have are shown below.

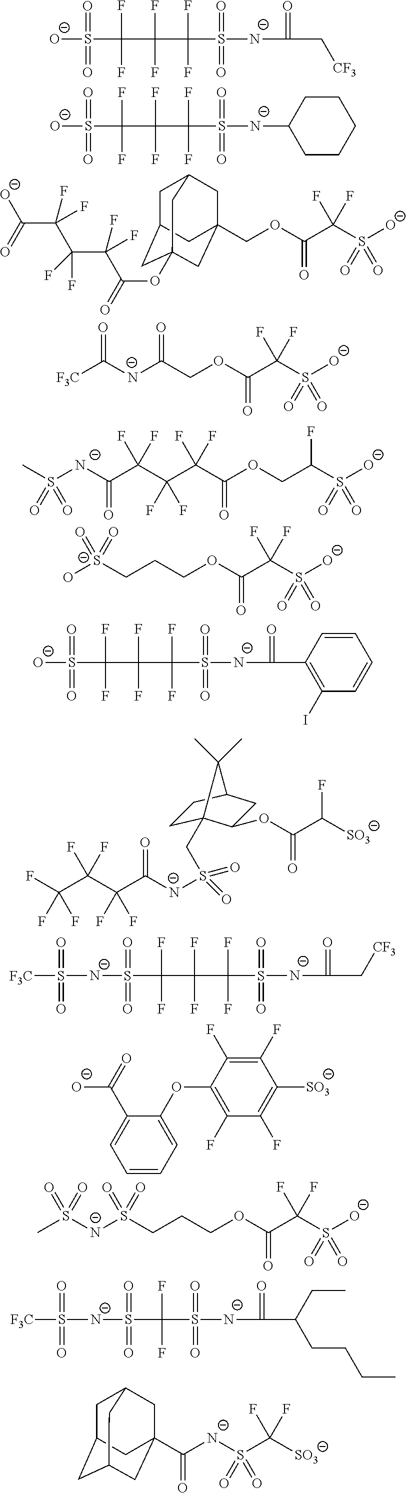

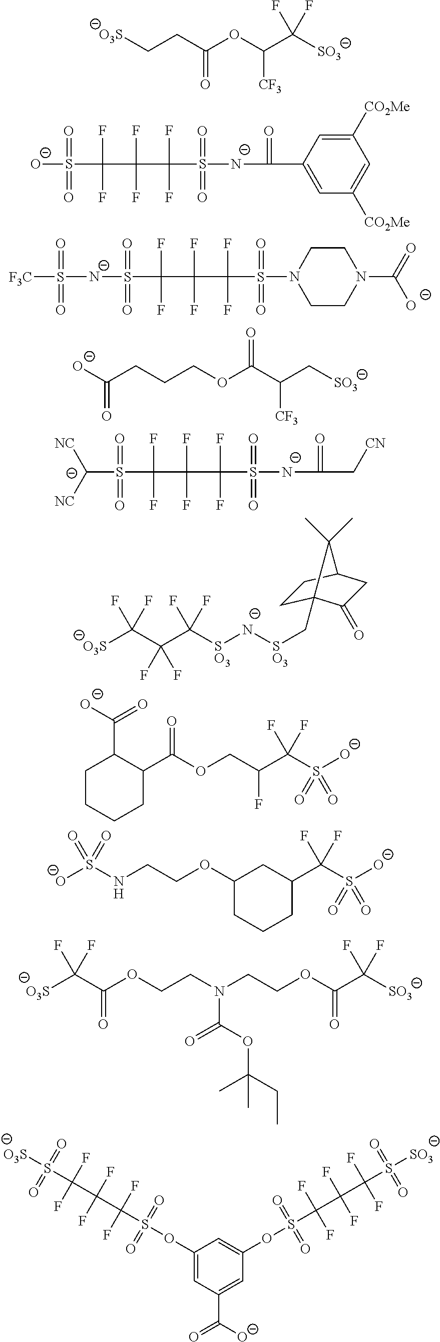

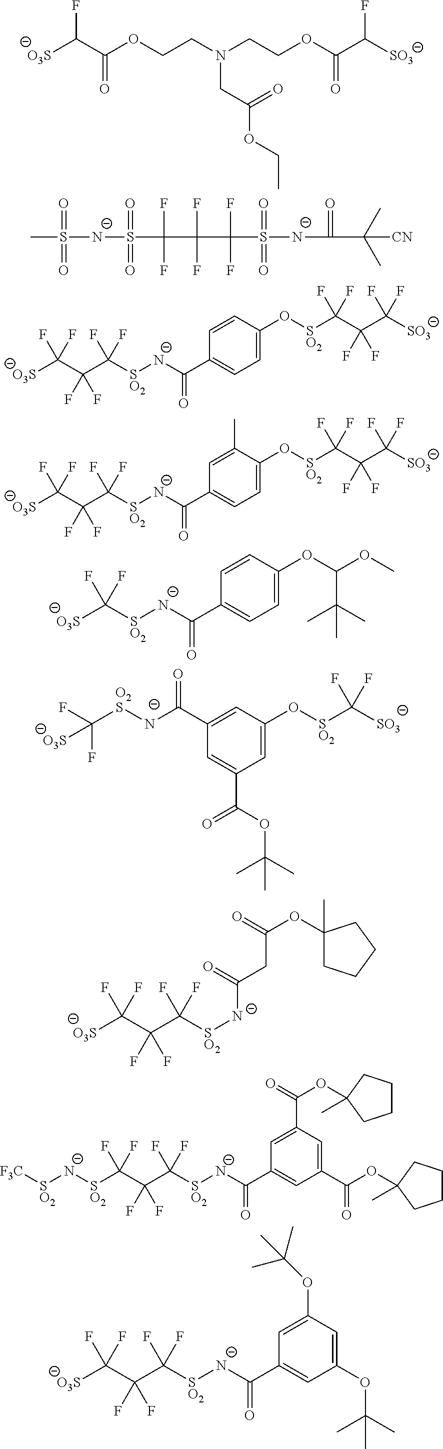

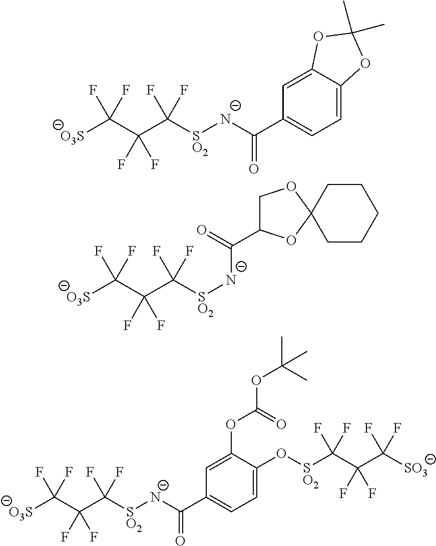

When the composition of the present invention includes the compound (C), the content of the compound (C) is not particularly limited, but is preferably 0.5 mass % or more, more preferably 1.0 mass % or more relative to the total solid contents of the composition of the present invention. When the composition of the present invention includes the compound (C), the content of the compound (C) is preferably 60.0 mass % or less, more preferably 50.0 mass % or less, still more preferably 40.0 mass % or less relative to the total solid contents of the composition of the present invention.

Such compounds (C) may be used alone or in combination of two or more thereof.

### [Acid Diffusion Control Agent]

The composition of the present invention may include an acid diffusion control agent.

The acid diffusion control agent acts as a quencher that traps an acid generated from the photoacid generator or the like upon exposure to suppress the reaction of the acid-decomposable resin in unexposed portions due to an excess of the generated acid.

The type of acid diffusion control agent is not particularly limited, and examples thereof include a basic compound (DA), a low-molecular-weight compound (DB) having a nitrogen atom and having a group that leaves due to the action of an acid, and a compound (DC) that undergoes a reduction or loss of the acid diffusion control ability upon irradiation with actinic rays or radiation.

Examples of the compound (DC) include an onium salt compound (DD) that becomes a weak acid relative to the photoacid generator, and a basic compound (DE) that undergoes a reduction or loss of the basicity upon irradiation with actinic rays or radiation.

Specific examples of the basic compound (DA) include those described in paragraphs [0132] to [0136] of WO2020/066824A. Specific examples of the basic compound (DE) that undergoes a reduction or loss of the basicity upon irradiation with actinic rays or radiation include those described in paragraphs [0137] to [0155] of WO2020/066824A and those described in paragraph [0164] of WO2020/066824A. Specific examples of the low-molecular-weight compound (DB) having a nitrogen atom and having a group that leaves due to the action of an acid include those described in paragraphs [0156] to [0163] of WO2020/066824A.

Specific examples of the onium salt compound (DD) that becomes a weak acid relative to the photoacid generator include those described in paragraphs [0305] to [0314] of WO2020/158337A.

In addition to the above, publicly known compounds disclosed in, for example, paragraphs [0627] to [0664] of US2016/0070167A, paragraphs [0095] to [0187] of US2015/0004544A, paragraphs [0403] to [0423] of US2016/0237190A, and paragraphs [0259] to [0328] of US2016/0274458A can be suitably used as the acid diffusion control agent.

Among the acid diffusion control agents, the compound (DC) is also referred to as a photodegradable quencher. The compound (DC) is a compound different from the compound (B). The acid diffusion control agent other than the compound (DC) is also referred to as a non-photodegradable quencher.

The acid diffusion control agent may be a photodegradable quencher or a non-photodegradable quencher. As the acid diffusion control agent, a photodegradable quencher and a non-photodegradable quencher may be used in combination.

When the composition of the present invention includes an acid diffusion control agent, the content of the acid diffusion control agent (in the case where a plurality of acid diffusion control agents are present, the total content) is preferably 0.1 to 30.0 mass %, more preferably 0.1 to 15.0 mass %, still more preferably 1.0 to 15.0 mass % relative to the total solid contents of the composition of the present invention.

In the composition of the present invention, such acid diffusion control agents may be used alone or in combination of two or more thereof

### [Hydrophobic Resin]

The composition of the present invention may further include a hydrophobic resin (also referred to as a “hydrophobic resin (I)”) different from the resin (A).

The hydrophobic resin is preferably designed so as to be localized in the surface of an actinic ray-sensitive or radiation-sensitive film (preferably a resist film) formed from the composition of the present invention. However, unlike surfactants, the hydrophobic resin does not necessarily need to have a hydrophilic group in the molecule, and does not necessarily contribute to homogeneous mixing of a polar substance and a nonpolar substance.

Advantages due to the addition of the hydrophobic resin may be control of static and dynamic contact angles at the surface of the resist film for water, and suppression of outgassing.

From the viewpoint of localization in the surface layer of the film, the hydrophobic resin preferably has one or more, more preferably two or more selected from the group consisting of a fluorine atom, a silicon atom, and a CH₃ partial structure included in a side chain moiety of the resin. The hydrophobic resin preferably has a hydrocarbon group having 5 or more carbon atoms. The resin may have such a group in the main chain thereof or, as a substituent, in a side chain thereof.

Examples of the hydrophobic resin include the compounds described in paragraphs [0275] to [0279] of WO2020/004306A.

Such hydrophobic resins may be used alone or in combination of two or more thereof.

When the composition of the present invention includes a hydrophobic resin, the content of the hydrophobic resin is preferably 0.01 to 20.0 mass %, more preferably 0.1 to 15.0 mass % relative to the total solid contents of the composition of the present invention.

### [Surfactant]

The composition of the present invention may include a surfactant. The composition including a surfactant enables the formation of a pattern having higher adhesiveness and less development defects.

The surfactant is preferably a fluorine-based and/or silicon-based surfactant.

Examples of the fluorine-based and/or silicon-based surfactant include the surfactants disclosed in paragraphs [0218] and [0219] of WO2018/193954A.

Such surfactants may be used alone or in combination of two or more thereof.

When the composition of the present invention includes a surfactant, the content of the surfactant is preferably 0.0001 to 2.0 mass %, more preferably 0.0005 to 1.0 mass %, still more preferably 0.1 to 1.0 mass % relative to the total solid contents of the composition of the present invention.

### [Solvent]

The composition of the present invention preferably includes a solvent.

The solvent preferably includes at least one of (M1) a propylene glycol monoalkyl ether carboxylate or (M2) at least one selected from the group consisting of propylene glycol monoalkyl ethers, lactates, acetates, alkoxypropionates, chain ketones, cyclic ketones, lactones, and alkylene carbonates. The solvent may further include a component other than the components (M1) and (M2).

Details of the component (M1) and the component (M2) are described in paragraphs [0218] to [0226] of WO2020/004306A, the contents of which are incorporated herein by reference.

When the solvent further includes a component other than the components (M1) and (M2), the content of the component other than the components (M1) and (M2) is preferably 5 to 30 mass % relative to the total amount of the solvent.

The content of the solvent in the composition of the present invention is determined such that the concentration of solid contents is preferably 0.5 to 30 mass %, more preferably 1 to 20 mass %. This further improves the coatability of the composition of the present invention.

Note that the solid contents refer to all components other than the solvent, and as described above, mean components that form an actinic ray-sensitive or radiation-sensitive film.

The concentration of solid contents is the mass percentage of the mass of the components other than the solvent relative to the total mass of the composition of the present invention.

The term “total solid contents” refers to the total mass of components excluding the solvent from the total composition of the composition of the present invention. The “solid contents” are components excluding the solvent as described above, and may be, for example, either solid or liquid at 25° C.

### [Other Additives]

The composition of the present invention may further include a dissolution inhibiting compound, a dye, a plasticizer, a photosensitizer, a light absorbent, and/or a compound that improves solubility in developers (for example, a phenolic compound having a molecular weight of 1,000 or less or an alicyclic or aliphatic compound including a carboxyl group).

The “dissolution inhibiting compound” refers to a compound that has a molecular weight of 3,000 or less and that is decomposed by the action of an acid to undergo a decrease in the degree of solubility in organic-based developers.

The composition of the present invention is suitably used as a photosensitive composition for EUV exposure.

EUV has a wavelength of 13.5 nm, which is shorter than the wavelength of ArF (wavelength 193 nm) light and the like, and thus provides a smaller number of incident photons when exposure is performed with the same sensitivity. Therefore, “photon shot noise”, which is stochastic variations in the number of photons, exerts a great influence, resulting in degradation of LER and bridge defects. In order to reduce the photon shot noise, a method of increasing the exposure dose to increase the number of incident photons may be employed; however, this method is a trade-off with the demand for higher sensitivity.

### [Actinic Ray-Sensitive or Radiation-Sensitive Film and Pattern Forming Method]

The present invention also relates to an actinic ray-sensitive or radiation-sensitive film formed using the composition of the present invention. The actinic ray-sensitive or radiation-sensitive film of the present invention is preferably a resist film.

The procedure of a pattern forming method using the composition of the present invention is not particularly limited, but the pattern forming method preferably has steps below.
Step 1: A step of forming an actinic ray-sensitive or radiation-sensitive film on a substrate using an actinic ray-sensitive or radiation-sensitive resin composition
Step 2: A step of exposing the actinic ray-sensitive or radiation-sensitive film
Step 3: A step of developing the exposed actinic ray-sensitive or radiation-sensitive film using a developer

The procedure of each of the steps will be described in detail below.

### (Step 1: Actinic Ray-Sensitive or Radiation-Sensitive Film Forming Step)

The step 1 is a step of forming an actinic ray-sensitive or radiation-sensitive film on a substrate using an actinic ray-sensitive or radiation-sensitive resin composition.

The method for forming an actinic ray-sensitive or radiation-sensitive film (preferably a resist film) on a substrate using the actinic ray-sensitive or radiation-sensitive resin composition may be, for example, a method of applying the composition of the present invention to a substrate.

If necessary, the composition of the present invention is preferably filtered through a filter before the application. The filter preferably has a pore size of 0.1 μm or less, more preferably 0.05 μm or less, still more preferably 0.03 μm or less. The filter is preferably formed of polytetrafluoroethylene, polyethylene, or nylon.

The composition of the present invention can be applied to a substrate (e.g., silicon, covered with silicon dioxide) as used in the production of integrated circuit elements by an appropriate coating method using a spinner, a coater, or the like. The coating method is preferably spin-coating using a spinner. The rotational speed during spin coating using a spinner is preferably 1,000 to 3,000 rpm (rotations per minute).

After application of the composition of the present invention, the substrate may be dried to form an actinic ray-sensitive or radiation-sensitive film. Note that, as needed, an underlying film (an inorganic film, an organic film, or an antireflection film) may be formed as an underlayer of the actinic ray-sensitive or radiation-sensitive film.

The drying method may be, for example, a method of performing drying by heating. The heating can be performed using means included in an ordinary exposure apparatus and/or an ordinary developing apparatus, or may be performed using a hot plate or the like. The heating temperature is preferably 80° C. to 150° C., more preferably 80° C. to 140° C., still more preferably 80° C. to 130° C. The heating time is preferably 30 to 1,000 seconds, more preferably 60 to 800 seconds, still more preferably 60 to 600 seconds.

The film thickness of the actinic ray-sensitive or radiation-sensitive film is not particularly limited, but is preferably 10 to 120 nm from the viewpoint that a fine pattern with higher accuracy can be formed. In particular, in the case of EUV exposure, the film thickness of the actinic ray-sensitive or radiation-sensitive film is more preferably 10 to 65 nm, still more preferably 15 to 50 nm. In the case of ArF liquid immersion exposure, the film thickness of the actinic ray-sensitive or radiation-sensitive film is more preferably 10 to 120 nm, still more preferably 15 to 90 nm.

A topcoat may be formed, as an overlying layer of the actinic ray-sensitive or radiation-sensitive film, using a topcoat composition.

Preferably, the topcoat composition does not mix with the actinic ray-sensitive or radiation-sensitive film and can be uniformly applied as an overlying layer of the actinic ray-sensitive or radiation-sensitive film. The topcoat is not particularly limited, a publicly known topcoat can be formed by a publicly known method, and, for example, a topcoat can be formed on the basis of the description of paragraphs [0072] to [0082] of JP2014-059543A.

For example, a topcoat including a basic compound and described in JP2013-61648A is preferably formed on the actinic ray-sensitive or radiation-sensitive film. Specific examples of the basic compound that can be included in the topcoat include the above-described basic compounds that may be included in the composition of the present invention.

The topcoat also preferably includes a compound including at least one group or bond selected from the group consisting of an ether bond, a thioether bond, a hydroxy group, a thiol group, a carbonyl bond, and an ester bond.

### (Step 2: Exposure Step)

The step 2 is a step of exposing the actinic ray-sensitive or radiation-sensitive film.

The exposure method may be a method of irradiating the formed actinic ray-sensitive or radiation-sensitive film with actinic rays or radiation through a predetermined mask.

Examples of the actinic rays or radiation include infrared light, visible light, ultraviolet light, far-ultraviolet light, extreme ultraviolet light, X-rays, and electron beams. A wavelength of 250 nm or less is preferred, a wavelength of 220 nm or less is more preferred, and far-ultraviolet light having a wavelength of 1 to 200 nm is particularly preferred. Specifically, KrF excimer laser (248 nm), ArF excimer laser (193 nm), F₂ excimer laser (157 nm), EUV (13.5 nm), X-rays, and an electron beam are particularly preferred.

After the exposure, baking (heating) is preferably performed before development. The baking accelerates the reaction in exposed portions to provide higher sensitivity and a better pattern shape.

The heating temperature is preferably 80° C. to 150° C., more preferably 80° C. to 140° C., still more preferably 80° C. to 130° C.

The heating time is preferably 10 to 1,000 seconds, more preferably 10 to 180 seconds, still more preferably 30 to 120 seconds.

The heating can be performed using means included in an ordinary exposure apparatus and/or an ordinary developing apparatus, or may be performed using a hot plate or the like.

This step is also referred to as post-exposure baking.

### (Step 3: Developing Step)

The step 3 is a step of developing the exposed actinic ray-sensitive or radiation-sensitive film using a developer to form a pattern.

The developer may be an alkali developer or a developer including an organic solvent (hereinafter, also referred to as an organic-based developer).

Examples of the developing method include a method of immersing the substrate for a predetermined time in a tank filled with a developer (dipping method), a method of puddling a developer over a surface of the substrate using surface tension and allowing the developer to stand for a predetermined time to perform development (puddling method), a method of spraying a developer onto a surface of the substrate (spraying method), and a method of continuously ejecting a developer over the substrate rotating at a constant rate, while scanning a developer ejection nozzle at a constant rate (dynamic dispensing method).

After the step of performing development, a step of stopping the development by performing replacement with another solvent may be performed.

The development time is not particularly limited as long as the resin in unexposed portions is sufficiently dissolved within the time, and is preferably 10 to 300 seconds, more preferably 20 to 120 seconds.

The temperature of the developer is preferably 0° C. to 50° C., more preferably 15° C. to 35° C.

The alkali developer used is preferably an alkaline aqueous solution including an alkali. The type of the alkaline aqueous solution is not particularly limited, but may be, for example, an alkaline aqueous solution including a quaternary ammonium salt typified by tetramethylammonium hydroxide, an inorganic alkali, a primary amine, a secondary amine, a tertiary amine, an alcoholamine, a cyclic amine, or the like. In particular, the alkali developer is preferably an aqueous solution of a quaternary ammonium salt typified by tetramethylammonium hydroxide (TMAH). An appropriate amount of an alcohol, a surfactant, or the like may be added to the alkali developer. Typically, the alkali developer preferably has an alkali concentration of 0.1 to 20 mass %. Typically, the alkali developer preferably has a pH of 10.0 to 15.0.

The organic-based developer is preferably a developer including at least one organic solvent selected from the group consisting of ketone-based solvents, ester-based solvents, alcohol-based solvents, amide-based solvents, ether-based solvents, and hydrocarbon-based solvents.

A plurality of such solvents may be mixed together, or such a solvent may be mixed with a solvent other than those described above or water. The developer as a whole preferably has a moisture content of less than 50 mass %, more preferably less than 20 mass %, still more preferably less than 10 mass %, particularly preferably contains substantially no moisture.

In the organic-based developer, the content of the organic solvent relative to the total amount of the developer is preferably 50 mass % or more and 100 mass % or less, more preferably 80 mass % or more and 100 mass % or less, still more preferably 90 mass % or more and 100 mass % or less, particularly preferably 95 mass % or more and 100 mass % or less.

### (Other Steps)

The pattern forming method preferably includes, after the step 3, a step of performing washing with a rinsing liquid.

The rinsing liquid used in the rinsing step after the developing step using the alkali developer may be, for example, pure water. Note that an appropriate amount of surfactant may be added to the pure water.

An appropriate amount of surfactant may be added to the rinsing liquid.

The rinsing liquid used in the rinsing step after the developing step using an organic-based developer is not particularly limited as long as it does not dissolve the pattern, and may be a solution including a typical organic solvent. The rinsing liquid used is preferably a rinsing liquid including at least one organic solvent selected from the group consisting of hydrocarbon-based solvents, ketone-based solvents, ester-based solvents, alcohol-based solvents, amide-based solvents, and ether-based solvents.

Examples of the method of performing the rinsing step include, but are not particularly limited to, a method of continuously ejecting a rinsing liquid onto the substrate rotating at a constant rate (spin-coating method), a method of immersing the substrate for a predetermined time in a tank filled with a rinsing liquid (dipping method), and a method of spraying a rinsing liquid onto a surface of the substrate (spraying method).

The pattern forming method may include a heating step (Post Bake) after the rinsing step. In this step, the developer and the rinsing liquid remaining between patterns and inside patterns are removed by baking. In addition, this step also provides an effect of annealing the resist pattern to improve the surface roughness of the pattern. The heating step after the rinsing step is performed usually at 40° C. to 250° C. (preferably 90° C. to 200° C.) for usually 10 seconds to 3 minutes (preferably 30 seconds to 120 seconds).

The formed pattern may be used as a mask to perform etching treatment of the substrate. Specifically, the pattern formed in the step 3 may be used as a mask to process the substrate (or the underlayer film and the substrate), thereby forming a pattern in the substrate.

The method of processing the substrate (or the underlayer film and the substrate) is not particularly limited, but is preferably a method of subjecting the substrate (or the underlayer film and the substrate) to dry etching using the pattern formed in the step 3 as a mask, thereby forming a pattern in the substrate. The dry etching is preferably oxygen plasma etching.

It is preferable that various materials used in the composition in the present specification and the pattern forming method in the present specification (for example, a solvent, a developer, a rinsing liquid, an antireflection film-forming composition, a topcoat-forming composition, and the like) do not include impurities such as metals. The content of impurities included in such materials is preferably 1 mass ppm (parts per million) or less, more preferably 10 mass ppb (parts per billion) or less, still more preferably 100 mass ppt (parts per trillion) or less, particularly preferably 10 mass ppt or less, most preferably 1 mass ppt or less. The lower limit is not particularly limited, and is preferably 0 mass ppt or more. Examples of the metal impurities include Na, K, Ca, Fe, Cu, Mg, Al, L₁, Cr, Ni, Sn, Ag, As, Au, Ba, Cd, Co, Pb, Ti, V, W, and Zn.

The method of removing impurities, such as metals, from the various materials may be, for example, filtration using a filter. Details of filtration using a filter are described in paragraph [0321] of WO2020/004306A.

Examples of a method for reducing the amount of impurities such as metals included in the various materials include a method of selecting, as raw materials constituting the various materials, raw materials having low metal contents, a method of subjecting raw materials constituting the various materials to filter filtration, and a method of performing distillation under conditions where contamination is reduced as much as possible by, for example, lining the inside of apparatuses with Teflon (registered trademark).

Instead of filter filtration, an adsorbing material may be used to remove impurities; alternatively, filter filtration and an adsorbing material may be used in combination. Publicly known adsorbing materials can be used as such adsorbing materials, and, for example, inorganic adsorbing materials such as silica gel and zeolite, and organic adsorbing materials such as activated carbon can be used. In order to reduce the amount of impurities such as metals included in the various materials, mixing of metal impurities in the production process needs to be prevented. Whether or not metal impurities are sufficiently removed from the production apparatuses can be checked by measuring the content of metal components included in a washing liquid that has been used for washing the production apparatuses. The content of metal components included in the washing liquid that has been used is preferably 100 mass ppt or less, more preferably 10 mass ppt or less, still more preferably 1 mass ppt or less. The lower limit is not particularly limited, and is preferably 0 mass ppt or more.

A conductive compound may be added to an organic treatment liquid such as a rinsing liquid in order to prevent failure of a chemical liquid pipe and various parts (such as a filter, an O-ring, and a tube) caused by electrostatic charging and subsequent electrostatic discharging. The conductive compound is not particularly limited, but may be, for example, methanol. The amount of addition is not particularly limited, but is preferably 10 mass % or less, more preferably 5 mass % or less from the viewpoint of maintaining preferred developing performance or rinsing performance. The lower limit is not particularly limited, and is preferably 0.01 mass % or more.

Examples of the chemical liquid pipe that can be used include various pipes made of SUS (stainless steel) or a material coated with polyethylene, polypropylene, or a fluororesin (such as polytetrafluoroethylene or a perfluoroalkoxy resin) subjected to antistatic treatment. Similarly, for the filter and the O-ring, polyethylene, polypropylene, or a fluororesin (such as polytetrafluoroethylene or a perfluoroalkoxy resin) subjected to antistatic treatment can be used.

### [Method for Producing Electronic Device]

The present specification also relates to a method for producing an electronic device, the method including the pattern forming method described above, and an electronic device produced by the production method.

In preferred embodiments of the electronic device in the present specification, the electronic device is mounted on electric or electronic devices (such as home appliances, OA (Office Automation), media-related devices, optical devices, and communication devices).

### EXAMPLES

Hereinafter, the present invention will be described in more detail with reference to Examples. Materials, amounts used, ratios, details of processes, and procedures of processes described in the following Examples may be appropriately changed without departing from the spirit of the present invention. Accordingly, the scope of the present invention should not be construed as being limited to the following Examples.

Various components used in resist compositions of Examples and Comparative Examples will be described below.

### [Resin (A)]

Resins A (resins A-1 to A-50) shown in Table 3 are described below.

The resins A used were synthesized in accordance with a method for synthesizing a resin A-1 (Synthesis Example 1) described later or a method for synthesizing a resin A-39 (Synthesis Example 2) described later.

Table 1 shows molar ratios of repeating units, the weight-average molecular weight (Mw), and the dispersity (Mw/Mn).

In Table 1, the molar ratio of each repeating unit represents the content (mol %) of the repeating unit relative to all the repeating units. Each repeating unit is shown by the structure of a monomer corresponding to the repeating unit.

The weight-average molecular weight (Mw) and the dispersity (Mw/Mn) of each of the resins A-1 to A-50 were (polystyrene-equivalent values) measured by GPC (carrier: tetrahydrofuran (THF)). The compositional ratios (mol % ratios) of the resins were measured by ¹³C-NMR (nuclear magnetic resonance).

TABLE 1

| | Structure of resin | | | | | | | | | |
|---|---|---|---|---|---|---|---|---|---|---|
| | Molar ratio of | | Molar ratio of | | Molar ratio of | | Molar ratio of | | | |
| | repeating unit 1 | | repeating unit 2 | | repeating unit 3 | | repeating unit 4 | | | |
| | | Content | | Content | | Content | | Content | | Mw/ |
| | Type | (mol %) | Type | (mol %) | Type | (mol %) | Type | (mol %) | Mw | Mn |
| Resin | MB-10 | 50 | MA-16 | 50 | — | — | — | — | 8500 | 1.60 |
| A-1 | | | | | | | | | | |
| Resin | MB-15 | 40 | MA-7 | 60 | — | — | — | — | 9000 | 1.70 |
| A-2 | | | | | | | | | | |
| Resin | MB-7 | 30 | MB-14 | 10 | MA-6 | 60 | — | — | 7000 | 1.55 |
| A-3 | | | | | | | | | | |
| Resin | MB-5 | 50 | MB-12 | 10 | MA-15 | 40 | — | — | 7500 | 1.55 |
| A-4 | | | | | | | | | | |
| Resin | MB-3 | 20 | MB-20 | 40 | MA-4 | 40 | — | — | 7000 | 1.60 |
| A-5 | | | | | | | | | | |
| Resin | MB-4 | 20 | MB-14 | 30 | MA-2 | 50 | — | — | 6500 | 1.63 |
| A-6 | | | | | | | | | | |
| Resin | MB-6 | 30 | MB-19 | 10 | MA-3 | 60 | — | — | 9500 | 1.45 |
| A-7 | | | | | | | | | | |
| Resin | MB-9 | 30 | MB-20 | 10 | MA-13 | 60 | — | — | 12000 | 1.65 |
| A-8 | | | | | | | | | | |
| Resin | MB-4 | 20 | MB-19 | 20 | MA-2 | 60 | — | — | 6000 | 1.55 |
| A-9 | | | | | | | | | | |
| Resin | MB-16 | 30 | MA-17 | 70 | — | — | — | — | 8000 | 1.40 |
| A-10 | | | | | | | | | | |
| Resin | MB-4 | 30 | MB-31 | 30 | MA-4 | 40 | — | — | 6500 | 1.65 |
| A-11 | | | | | | | | | | |
| Resin | MB-30 | 20 | MA-8 | 80 | — | — | — | — | 5500 | 1.65 |
| A-12 | | | | | | | | | | |
| Resin | MB-13 | 50 | MA-10 | 50 | — | — | — | — | 15000 | 1.75 |
| A-13 | | | | | | | | | | |
| Resin | MB-8 | 30 | MA-20 | 70 | — | — | — | — | 9000 | 1.60 |
| A-14 | | | | | | | | | | |
| Resin | MB-18 | 30 | MB-29 | 30 | MA-14 | 40 | — | — | 8000 | 1.55 |
| A-15 | | | | | | | | | | |
| Resin | MB-3 | 30 | MB-20 | 20 | MA-2 | 40 | MA-8 | 10 | 7500 | 1.70 |
| A-16 | | | | | | | | | | |
| Resin | MB-1 | 30 | MB-26 | 30 | MA-11 | 40 | — | — | 18000 | 1.80 |
| A-17 | | | | | | | | | | |
| Resin | MB-27 | 60 | MA-5 | 40 | — | — | — | — | 7500 | 1.65 |
| A-18 | | | | | | | | | | |
| Resin | MB-17 | 30 | MB-24 | 10 | MA-19 | 60 | — | — | 8000 | 1.70 |
| A-19 | | | | | | | | | | |
| Resin | MB-11 | 30 | MB-28 | 40 | MA-9 | 30 | — | — | 9500 | 1.80 |
| A-20 | | | | | | | | | | |

TABLE 2

| | Structure of resin | | | | | | | | | |
|---|---|---|---|---|---|---|---|---|---|---|
| | Molar ratio of | | Molar ratio of | | Molar ratio of | | Molar ratio of | | | |
| | repeating unit 1 | | repeating unit 2 | | repeating unit 3 | | repeating unit 4 | | | |
| | | Content | | Content | | Content | | Content | | Mw/ |
| | Type | (mol %) | Type | (mol %) | Type | (mol %) | Type | (mol %) | Mw | Mn |
| Resin A-21 | MB-3 | 20 | MB-20 | 10 | MB-32 | 10 | MA-2 | 60 | 10000 | 1.70 |
| Resin A-22 | MB-2 | 60 | MA-1 | 40 | — | — | — | — | 11000 | 1.65 |
| Resin A-23 | MB-21 | 50 | MA-12 | 50 | — | — | — | — | 6500 | 1.60 |
| Resin A-24 | MB-23 | 40 | MA-10 | 60 | — | — | — | — | 8000 | 1.55 |
| Resin A-25 | MB-1 | 20 | MB-19 | 20 | MA-2 | 60 | — | — | 8000 | 1.55 |
| Resin A-26 | MB-25 | 30 | MA-2 | 70 | — | — | — | — | 7500 | 1.60 |
| Resin A-27 | MB-3 | 40 | MA-3 | 60 | — | — | — | — | 9500 | 1.60 |
| Resin A-28 | MB-22 | 40 | MA-4 | 60 | — | — | — | — | 10000 | 1.70 |
| Resin A-29 | MB-3 | 30 | MB-33 | 20 | MA-2 | 50 | — | — | 6600 | 1.63 |
| Resin A-30 | MB-7 | 30 | MB-14 | 10 | MA-18 | 60 | — | — | 8100 | 1.65 |
| Resin A-31 | MB-1 | 20 | MB-19 | 20 | MA-21 | 60 | — | — | 6600 | 1.65 |
| Resin A-32 | MB-17 | 60 | MA-24 | 40 | — | — | — | — | 8200 | 1.71 |
| Resin A-33 | MB-4 | 20 | MB-19 | 20 | MA-23 | 60 | — | — | 6500 | 1.60 |
| Resin A-34 | MB-14 | 30 | MA-22 | 70 | — | — | — | — | 7200 | 1.58 |
| Resin A-35 | MB-19 | 25 | MB-34 | 25 | MA-2 | 50 | — | — | 8400 | 1.59 |
| Resin A-36 | MB-3 | 30 | MB-20 | 10 | MA-5 | 60 | — | — | 12000 | 1.60 |
| Resin A-37 | MB-3 | 10 | MB-13 | 30 | MB-20 | 10 | MA-2 | 50 | 18000 | 1.70 |
| Resin A-38 | MB-3 | 10 | MB-20 | 20 | MA-2 | 50 | MA-11 | 20 | 6000 | 1.55 |
| Resin A-39 | MB-3 | 10 | MB-20 | 30 | MA-2 | 60 | — | — | 10000 | 1.35 |
| Resin A-40 | MB-35 | 50 | MA-25 | 50 | — | — | — | — | 8000 | 1.55 |

TABLE 3

| | Structure of resin | | | | | | | | | |
|---|---|---|---|---|---|---|---|---|---|---|
| | Molar ratio of | | Molar ratio of | | Molar ratio of | | Molar ratio of | | | |
| | repeating unit 1 | | repeating unit 2 | | repeating unit 3 | | repeating unit 4 | | | |
| | | Content | | Content | | Content | | Content | | Mw/ |
| | Type | (mol %) | Type | (mol %) | Type | (mol %) | Type | (mol %) | Mw | Mn |
| Resin A-41 | MB-35 | 30 | MB-13 | 15 | MA-25 | 55 | — | — | 9200 | 1.71 |
| Resin A-42 | MB-3 | 10 | MB-20 | 20 | MA-2 | 70 | — | — | 11000 | 1.58 |
| Resin A-43 | MB-4 | 10 | MB-19 | 20 | MA-3 | 50 | MA-11 | 20 | 8800 | 1.56 |
| Resin A-44 | MB-3 | 20 | MB-20 | 20 | MA-5 | 60 | — | — | 10000 | 1.70 |
| Resin A-45 | MB-3 | 10 | MB-13 | 10 | MB-20 | 20 | MA-2 | 60 | 8500 | 1.48 |
| Resin A-46 | MB-17 | 40 | MA-24 | 40 | MA-7 | 20 | — | — | 12100 | 1.72 |
| Resin A-47 | MB-18 | 15 | MB-19 | 30 | MA-3 | 55 | — | — | 6700 | 1.57 |
| Resin A-48 | MB-5 | 25 | MB-19 | 10 | MB-21 | 10 | MA-4 | 55 | 8600 | 1.52 |
| Resin A-49 | MB-22 | 20 | MB-6 | 10 | MA-13 | 20 | MA-7 | 50 | 9800 | 1.66 |
| Resin A-50 | MB-25 | 10 | MB-28 | 15 | MB-1 | 20 | MA-4 | 55 | 11500 | 1.65 |

The structures of MA-1 to MA-25 and MB-1 to MB-35, which are monomers corresponding to the repeating units shown in Table 1, are shown below.

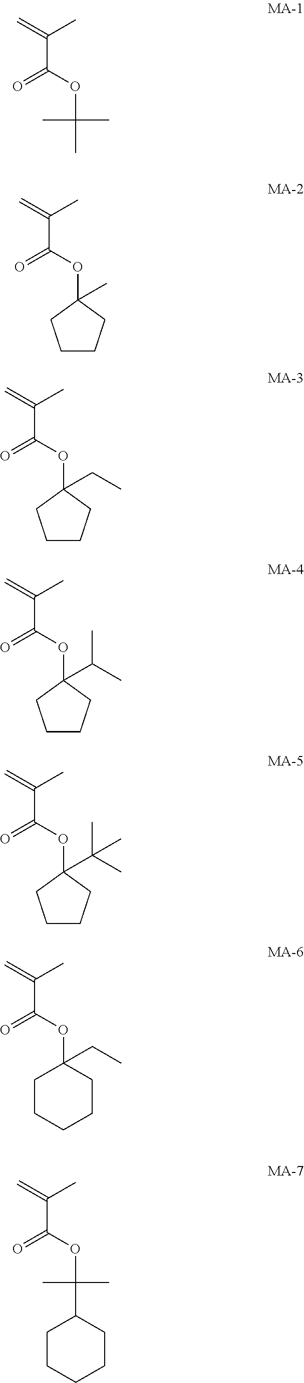

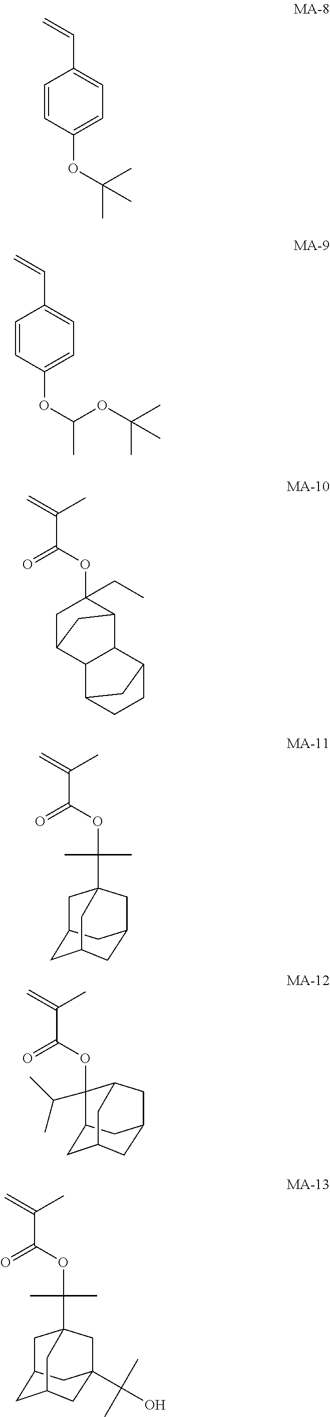

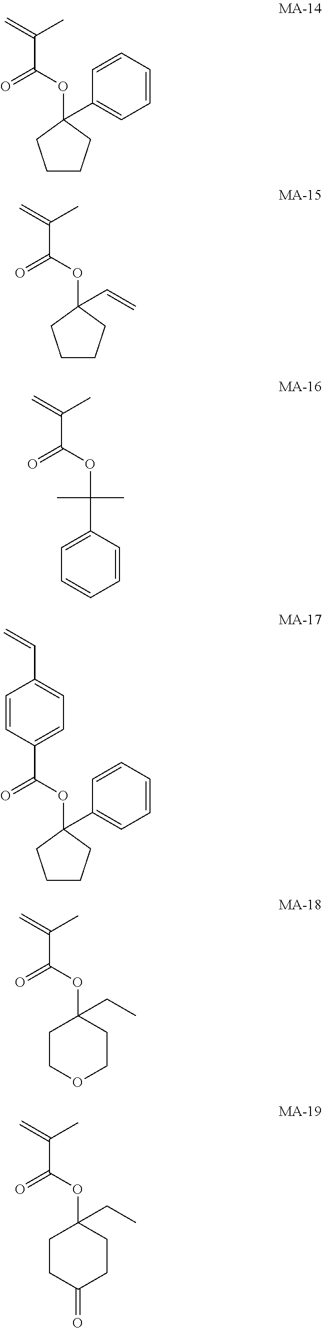

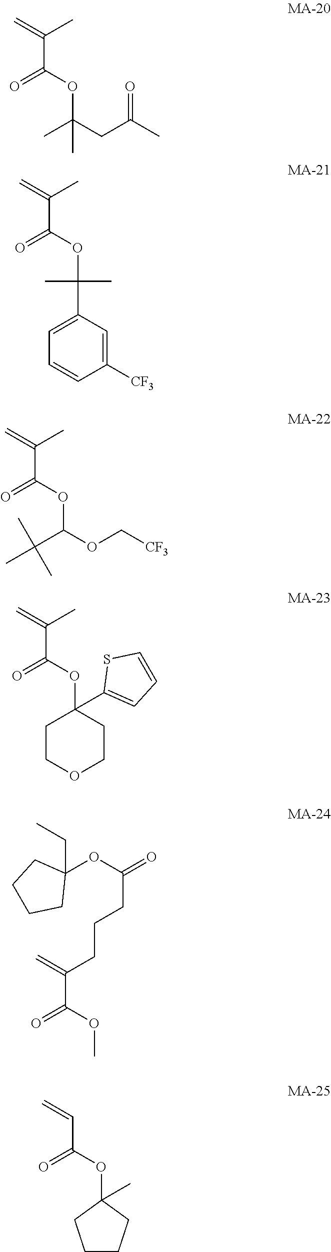

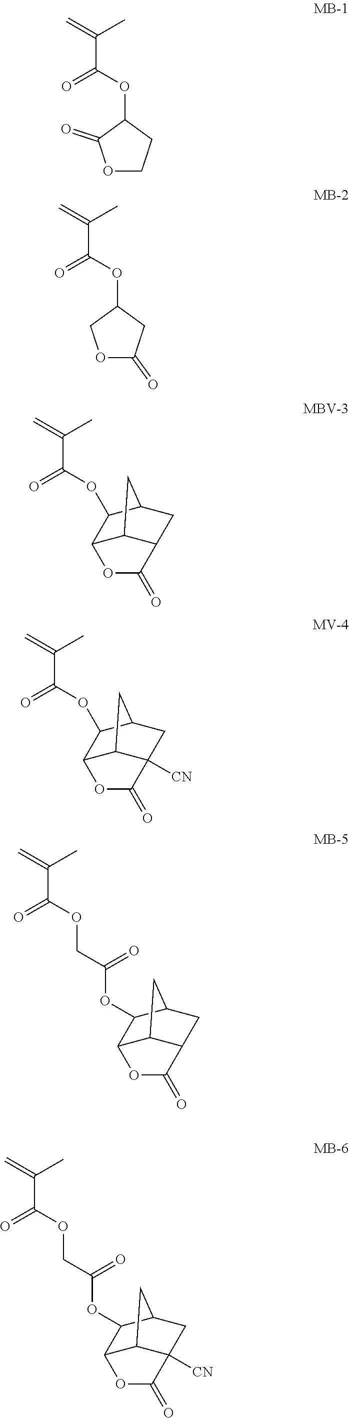

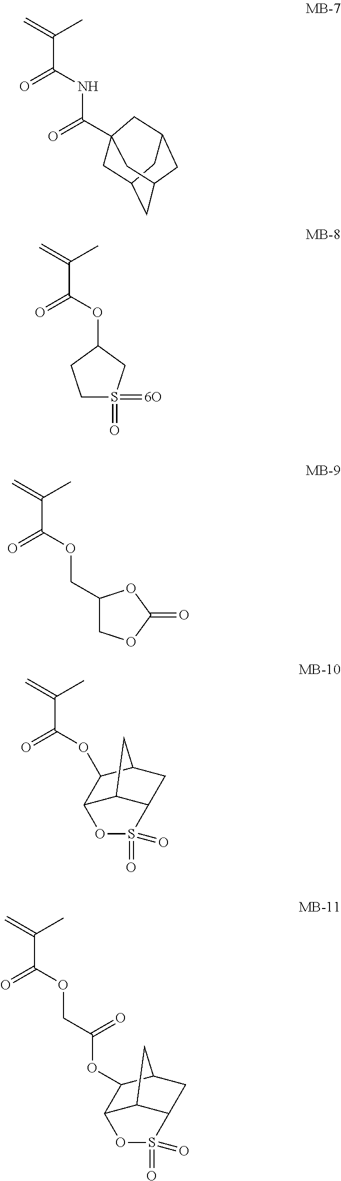

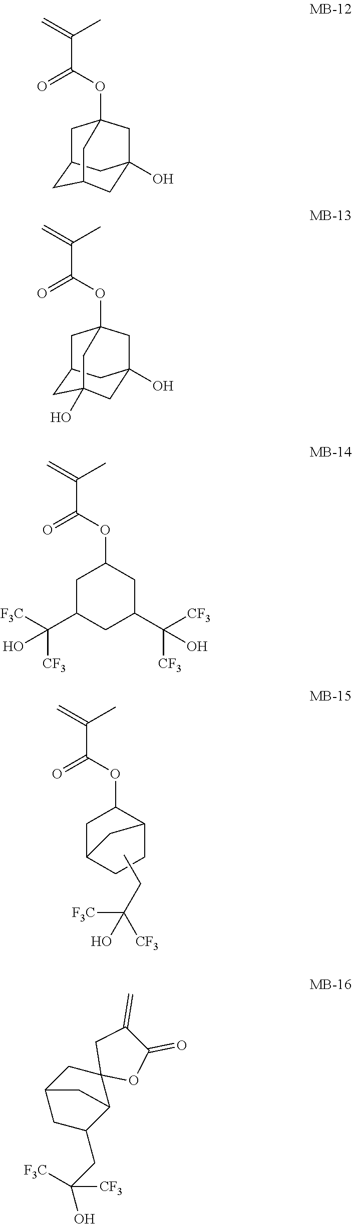

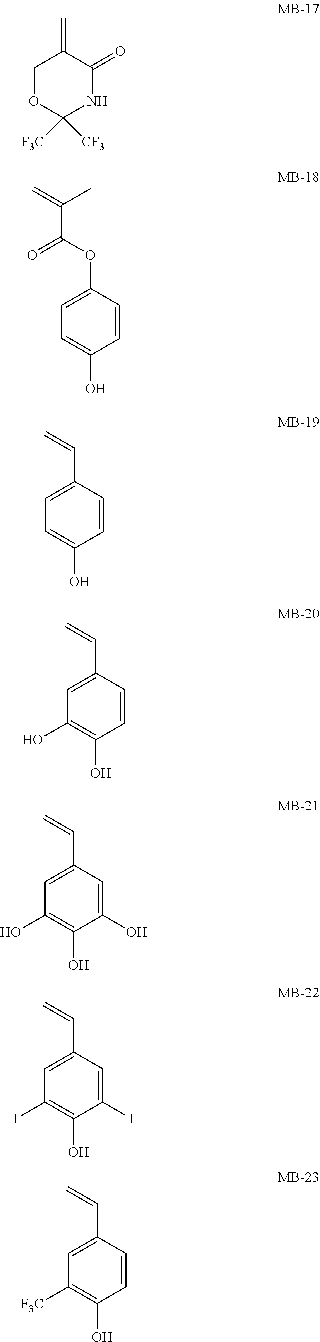

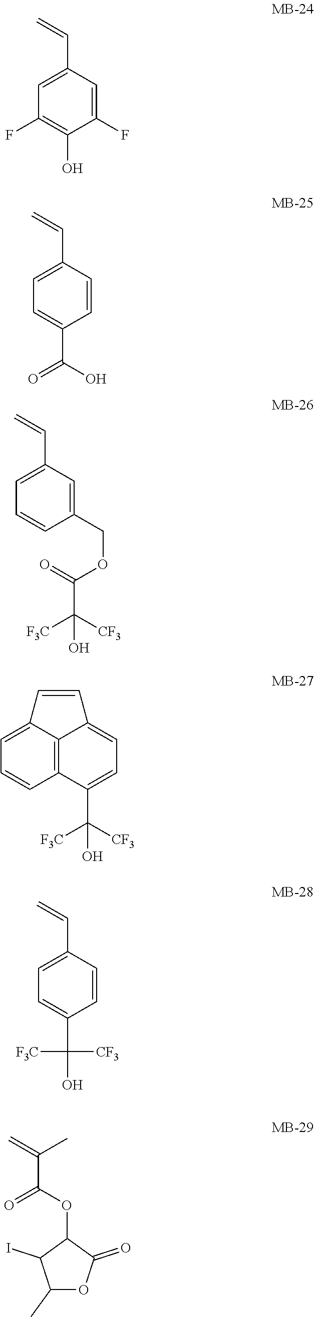

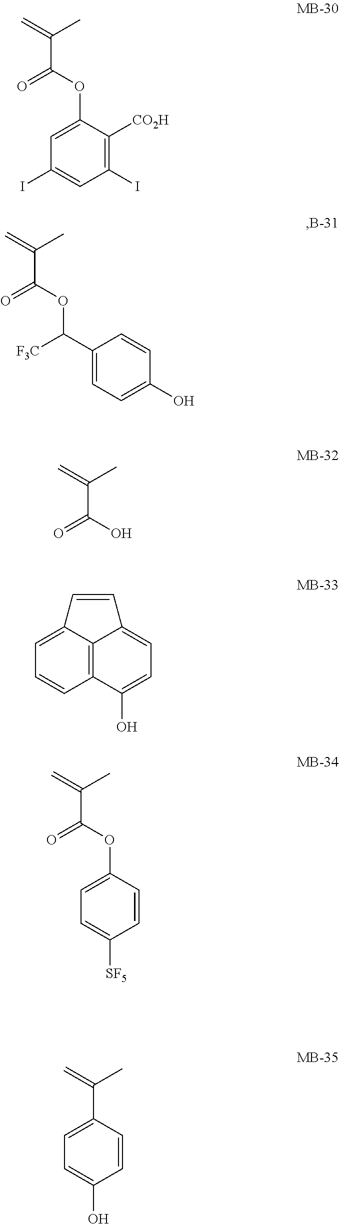

### Synthesis Example 1: Synthesis of Resin A-1

Propylene glycol monomethyl ether acetate (28 g) was heated to 80° C. under a nitrogen stream. While this liquid was stirred, a mixed solution of the monomer represented by MA-16 (30 g), the monomer represented by MB-10 (38 g), propylene glycol monomethyl ether acetate (112 g), and dimethyl 2,2′-azobisisobutyrate [V-601, manufactured by FUJIFILM Wako Pure Chemical Corporation] (5.7 g) was added dropwise thereto over six hours to obtain a reaction liquid. After completion of the dropwise addition, the reaction liquid was further stirred at 80° C. for two hours. The obtained reaction liquid was left to cool, then reprecipitated with a large amount of mixed solvent of heptane and ethyl acetate (heptane:ethyl acetate=9:1, mass ratio), and then filtered. The obtained solid was vacuum-dried to obtain 58 g of the resin A-1. The resin A-1 had a weight-average molecular weight (Mw: polystyrene equivalent) of 8,500 and a dispersity (Mw/Mn) of 1.60 as determined by GPC (carrier: tetrahydrofuran (THF)). The molar ratio of the repeating units measured by ¹³C-NMR (nuclear magnetic resonance) was MB-10/MA-16=50/50.

### Synthesis Example 2: Synthesis of Resin A-39

Propylene glycol monomethyl ether acetate (22 g) was heated to 85° C. under a nitrogen stream. While this liquid was stirred, a mixed solution of the monomer represented by MB-3 (6 g), a monomer represented by MB-20a (35 g), the monomer represented by MA-2 (27 g), propylene glycol monomethyl ether acetate (84 g), dimethyl 2,2′-azobisisobutyrate [V-601, manufactured by FUJIFILM Wako Pure Chemical Corporation] (1.2 g), and methyl 4-cyano-4-[(dodecylsulfanylthiocarbonyl)sulfanyl]pentanoate [manufactured by FUJIFILM Wako Pure Chemical Corporation] (4.4 g) was added dropwise thereto over six hours. After completion of the dropwise addition, the reaction liquid was further stirred at 85° C. for two hours to obtain a polymerization solution.

To the polymerization solution, methanol (100 g) and triethylamine (16 g) were added, and the resulting solution was stirred at 50° C. for five hours. After completion of stirring, the solution was left to cool to room temperature, ethyl acetate (650 g) and a 0.2 mol/L aqueous hydrochloric acid solution (400 mL) were then added, followed by stirring for 30 minutes, and the organic layer was extracted. The extracted organic layer was washed five times with distilled water (400 mL). The washed organic layer was reprecipitated with a mixed solution of heptane/ethyl acetate=9/1 (mass ratio), and then filtered. The obtained solid was vacuum-dried to obtain 38 g of the resin A-39.

The resin A-39 had a weight-average molecular weight (Mw: polystyrene equivalent) of 10,000 and a dispersity (Mw/Mn) of 1.35 as determined by GPC (carrier: tetrahydrofuran (THF)). The molar ratio of the repeating units measured by ¹³C-NMR (nuclear magnetic resonance) was MB-3/MB-20/MA-2=10/30/60.

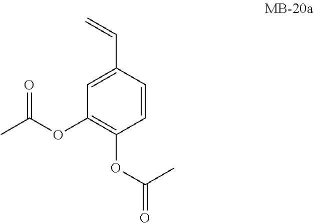

The resins A-2 to A-38 and the resins A-40 to A-50 used were synthesized in accordance with the method for synthesizing the resin A-1 (Synthesis Example 1).

### [Photoacid Generator (B)]

Compounds B-1 to B-40 were used as the photoacid generator (B).

In a Comparative Example, a photoacid generator Ba-1, which was not the photoacid generator (B), was used. For convenience, Ba-1 is also described in the column of the photoacid generator (B) in Table 3 below.

Numerical values described in the following structures represent values of acid dissociation constants (pKa) derived from each acidic moiety of a compound obtained by replacing cationic moieties with H⁺, the values being determined by calculation using the following software package 1.

Software package 1: Advanced Chemistry Development (ACD/Labs) Software V8.14 for Solaris (1994-2007 ACD/Labs)

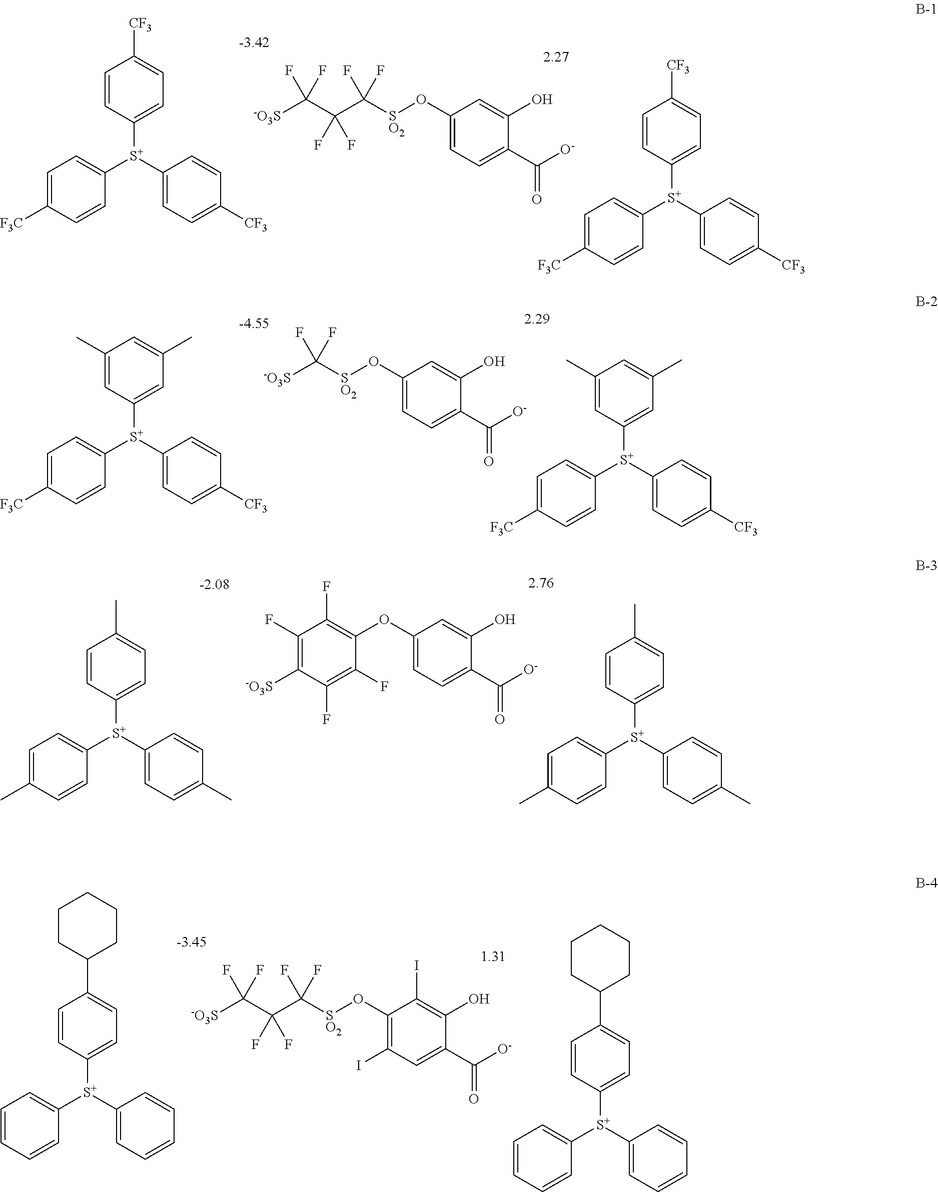

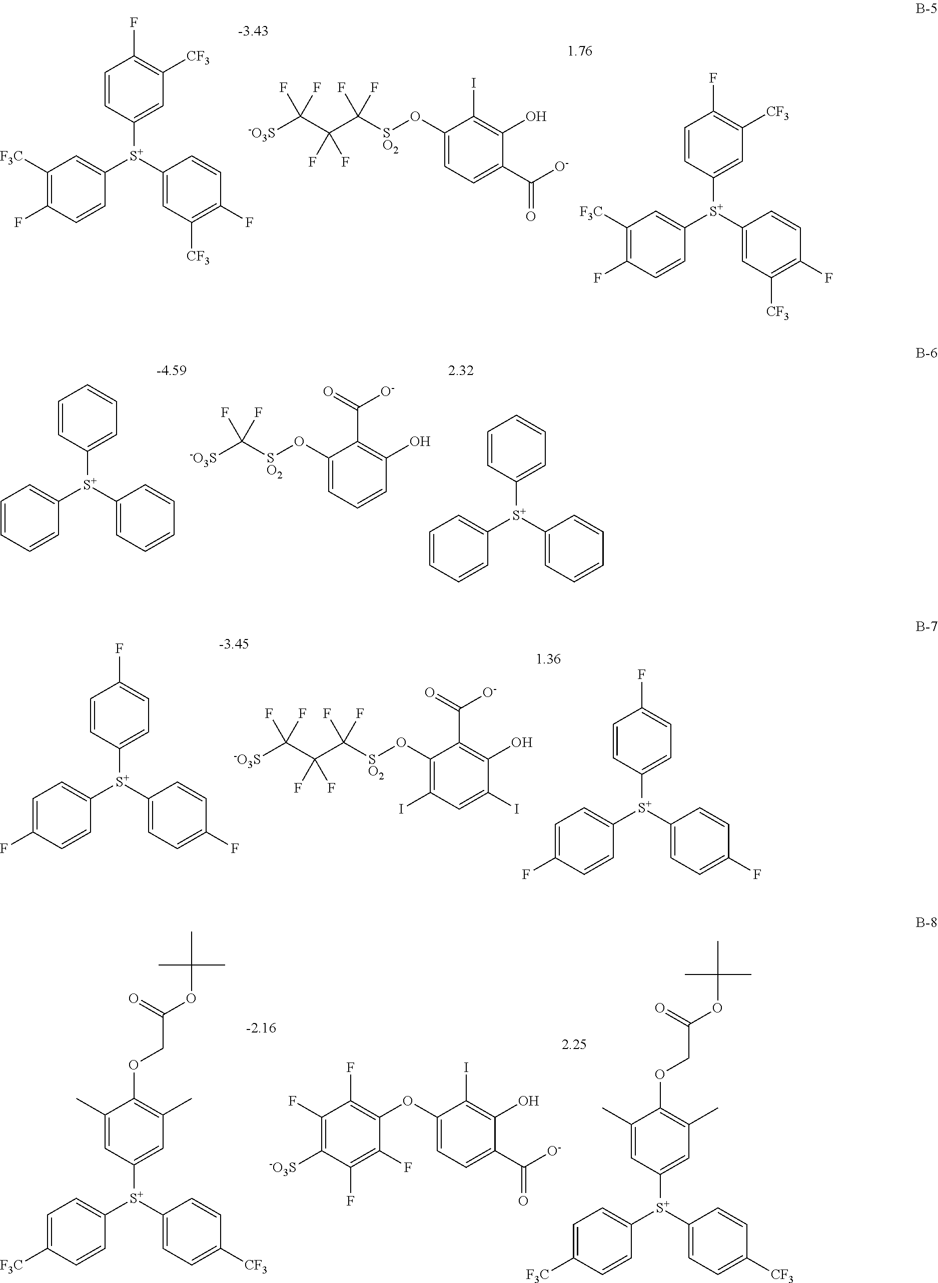

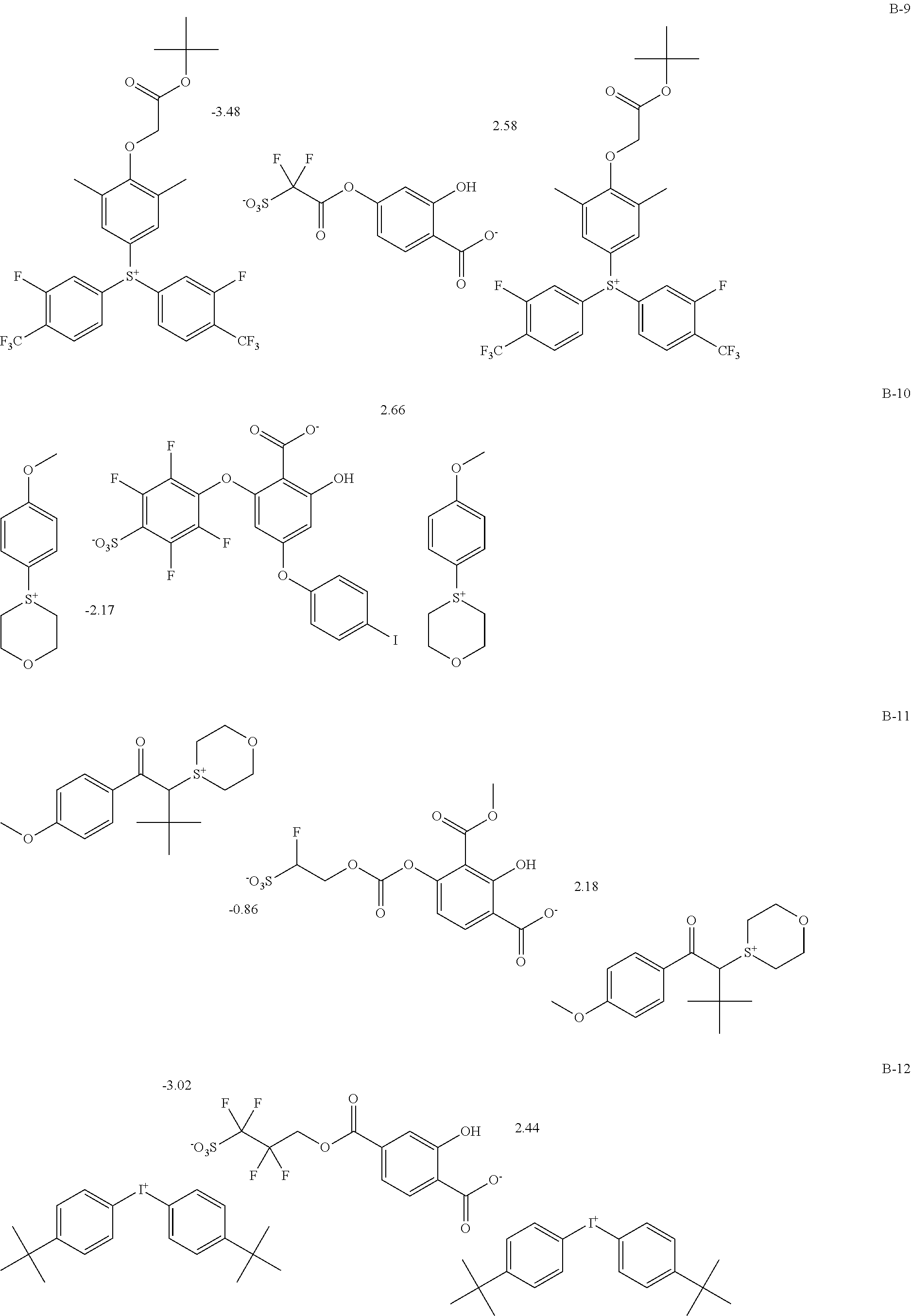

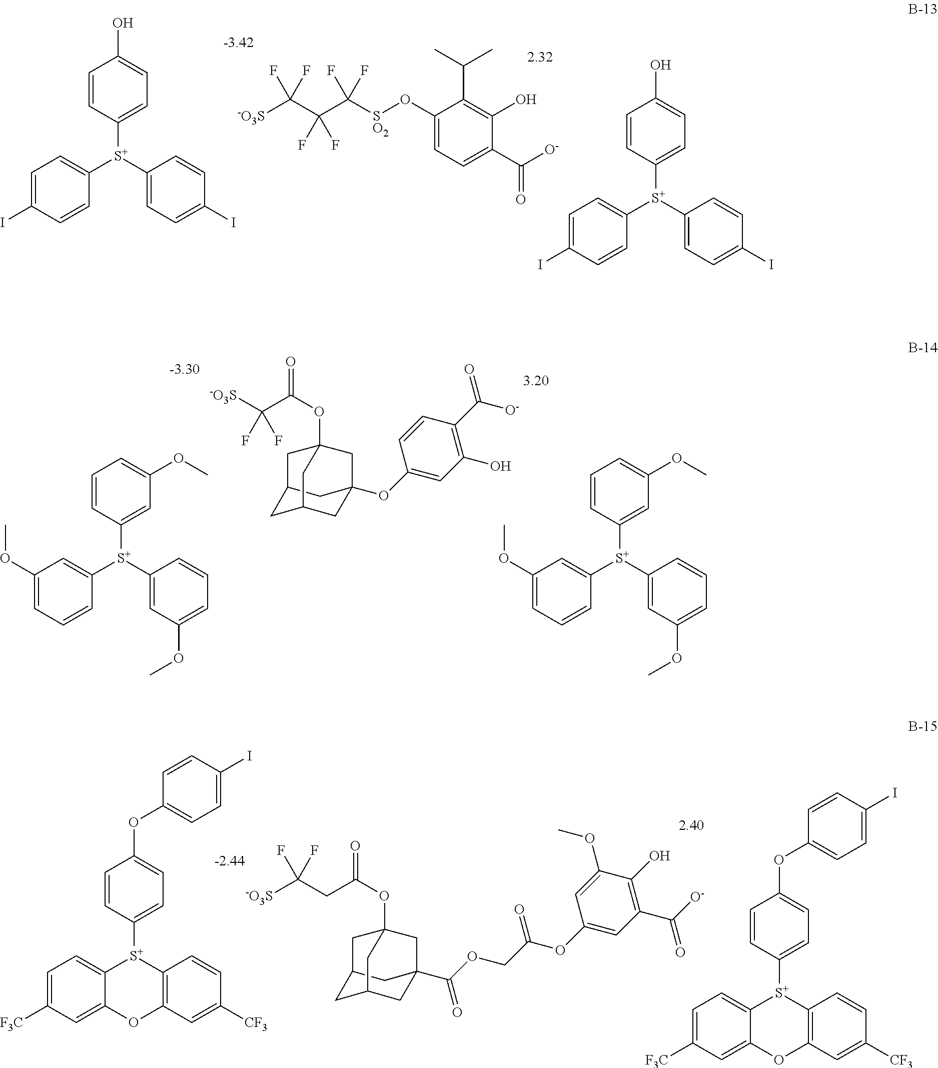

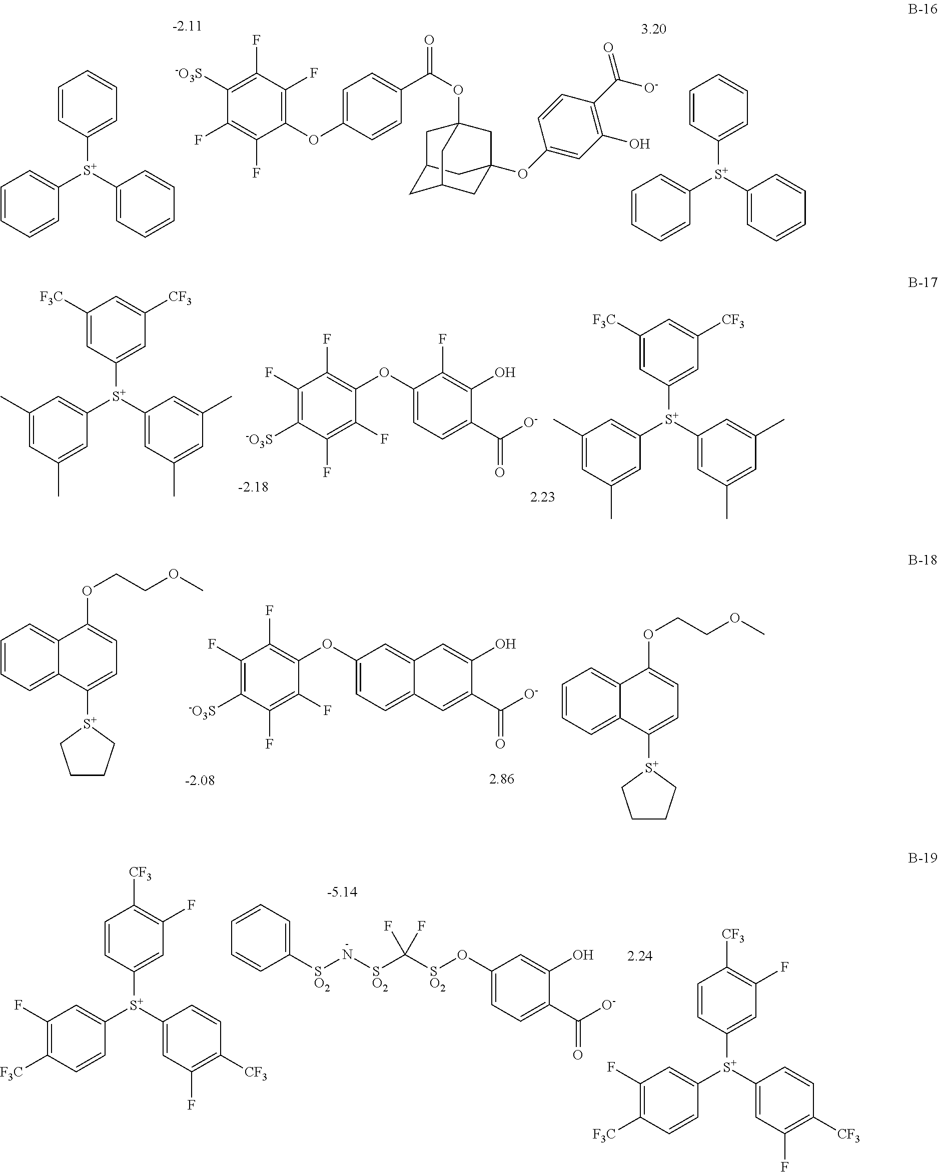

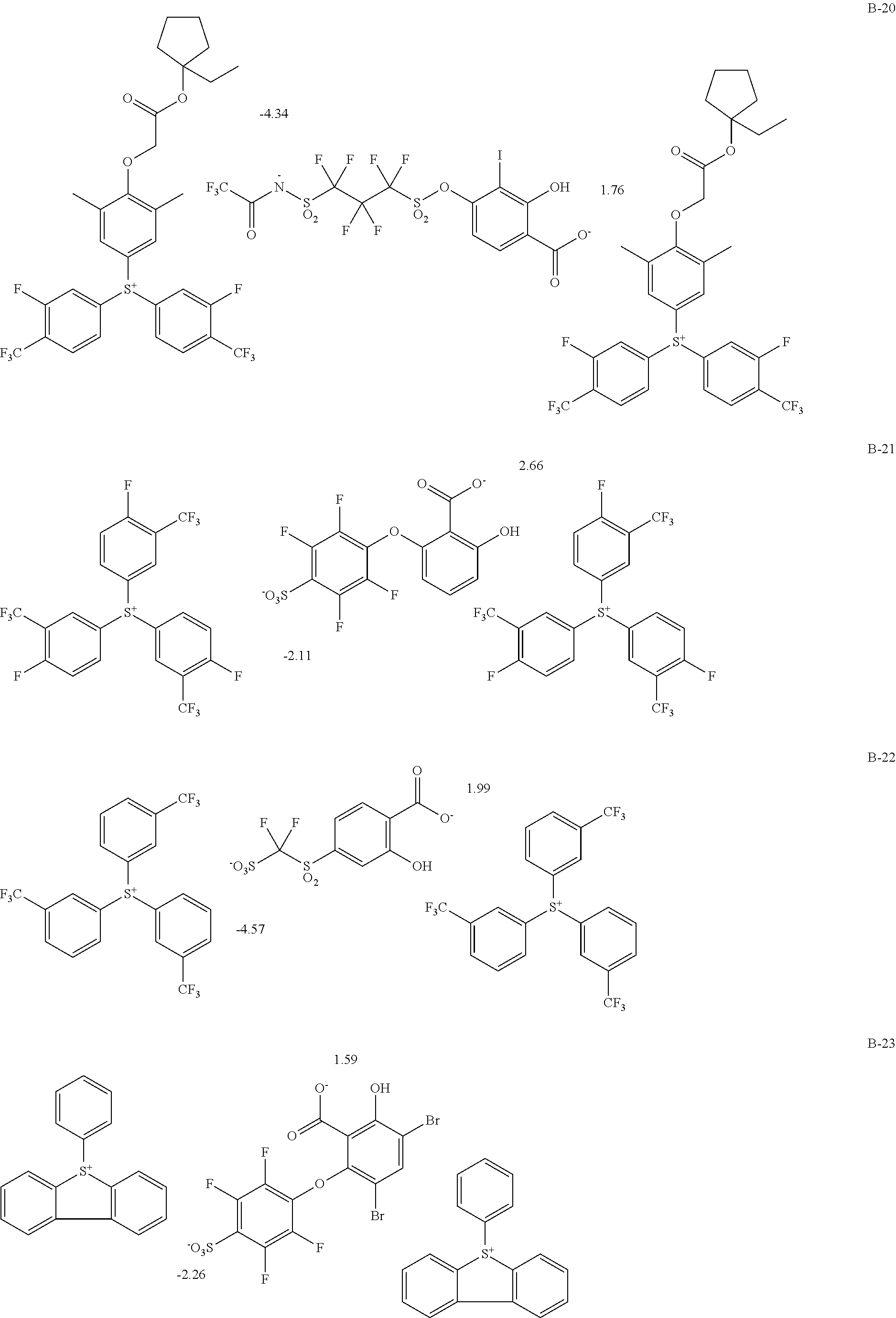

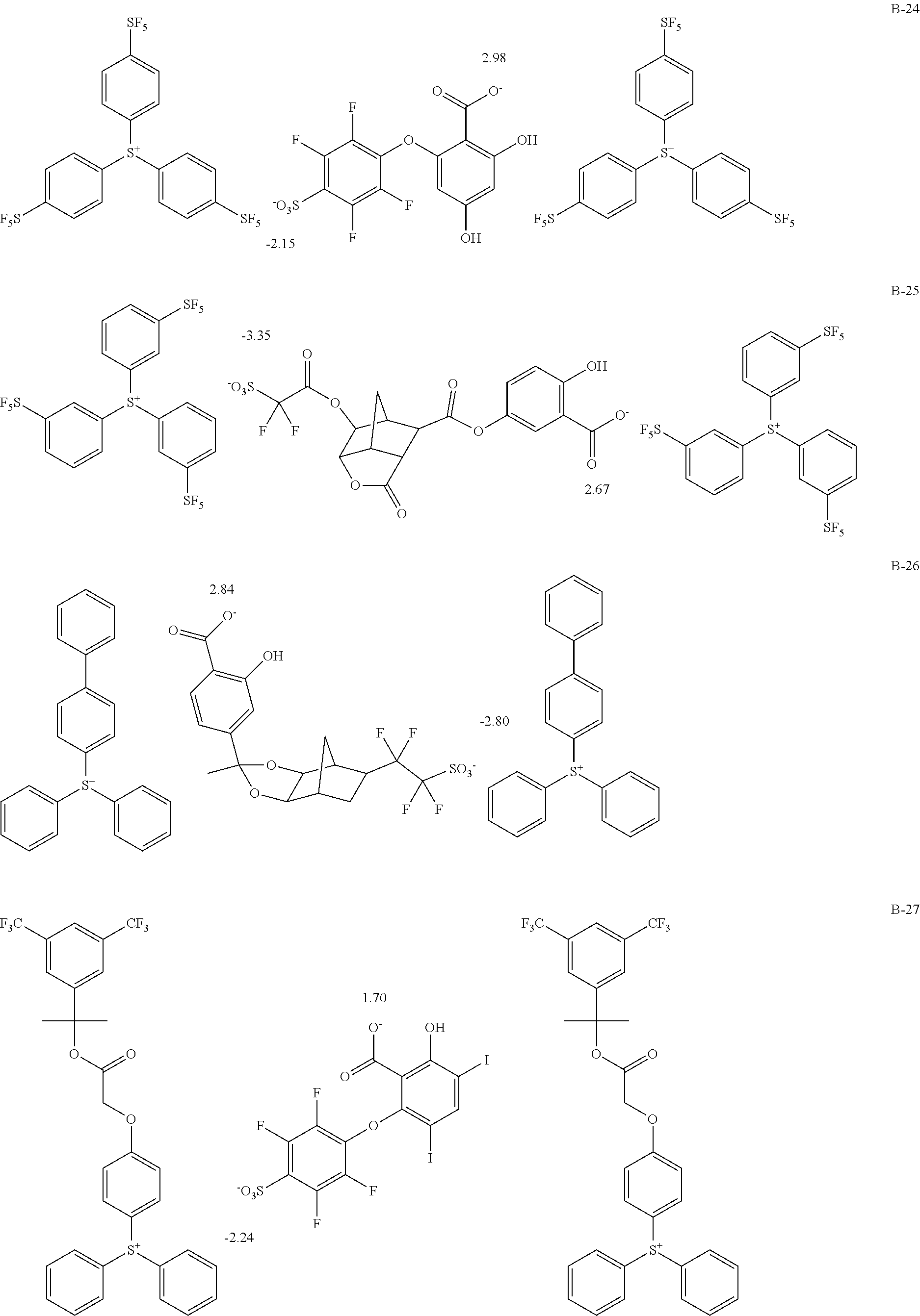

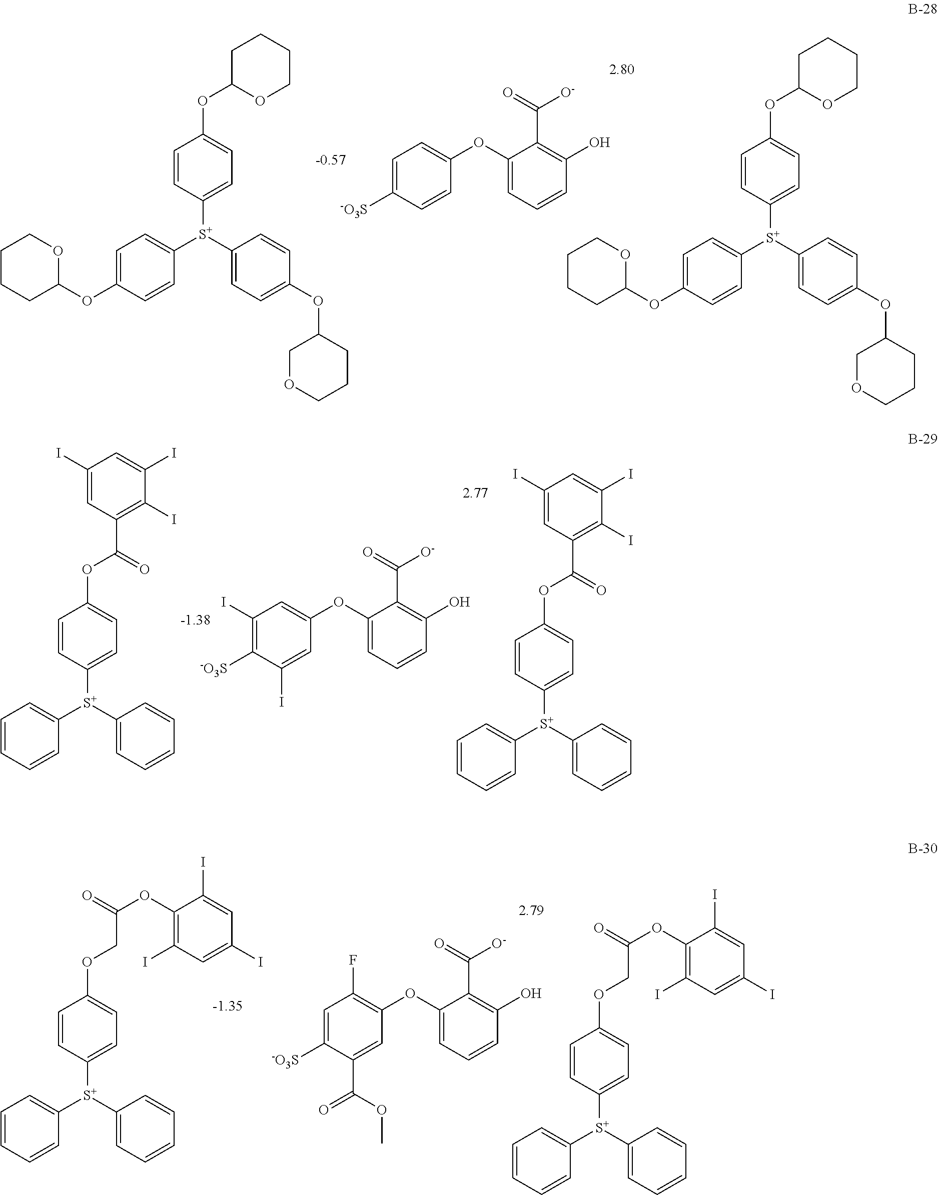

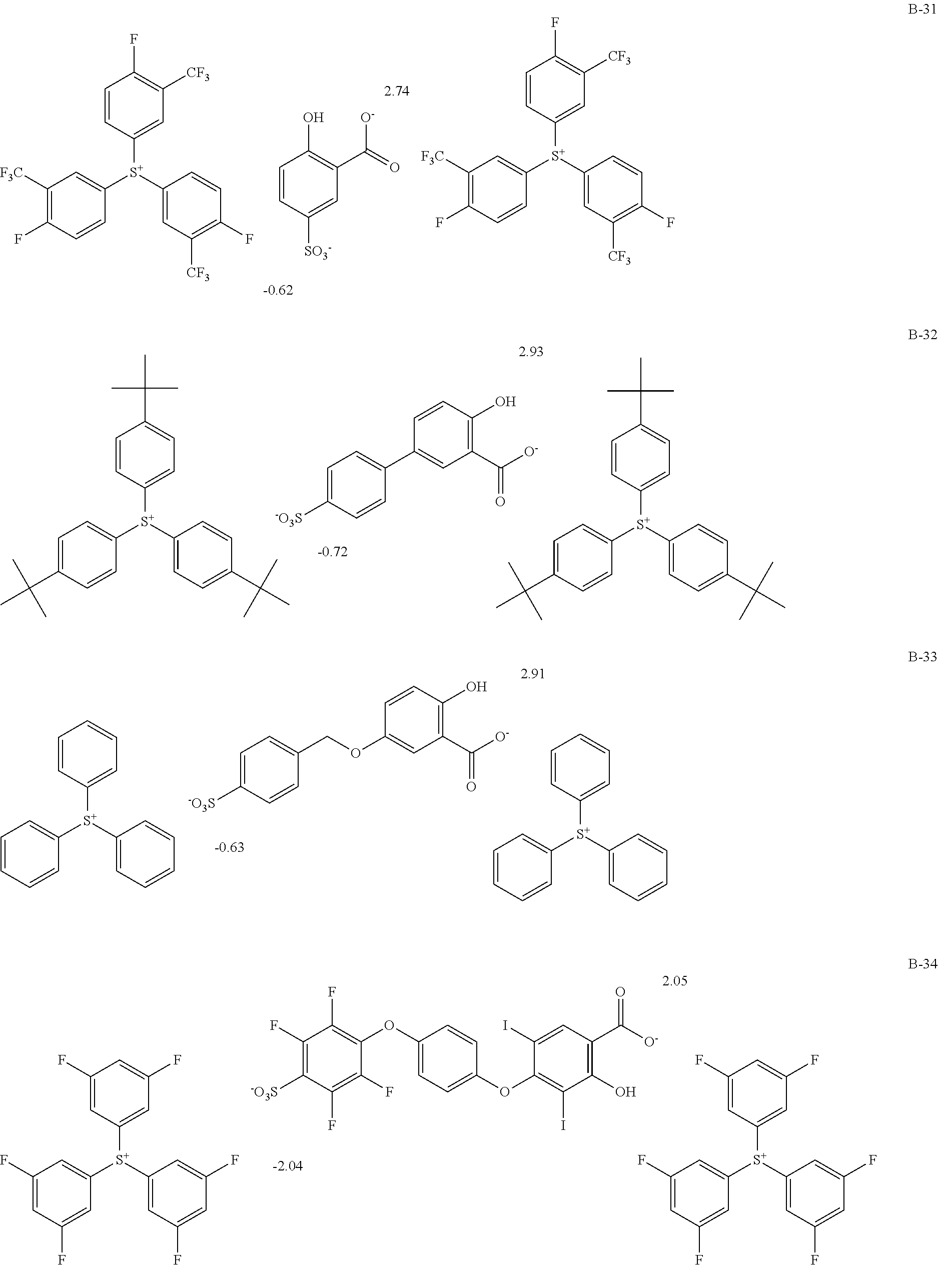

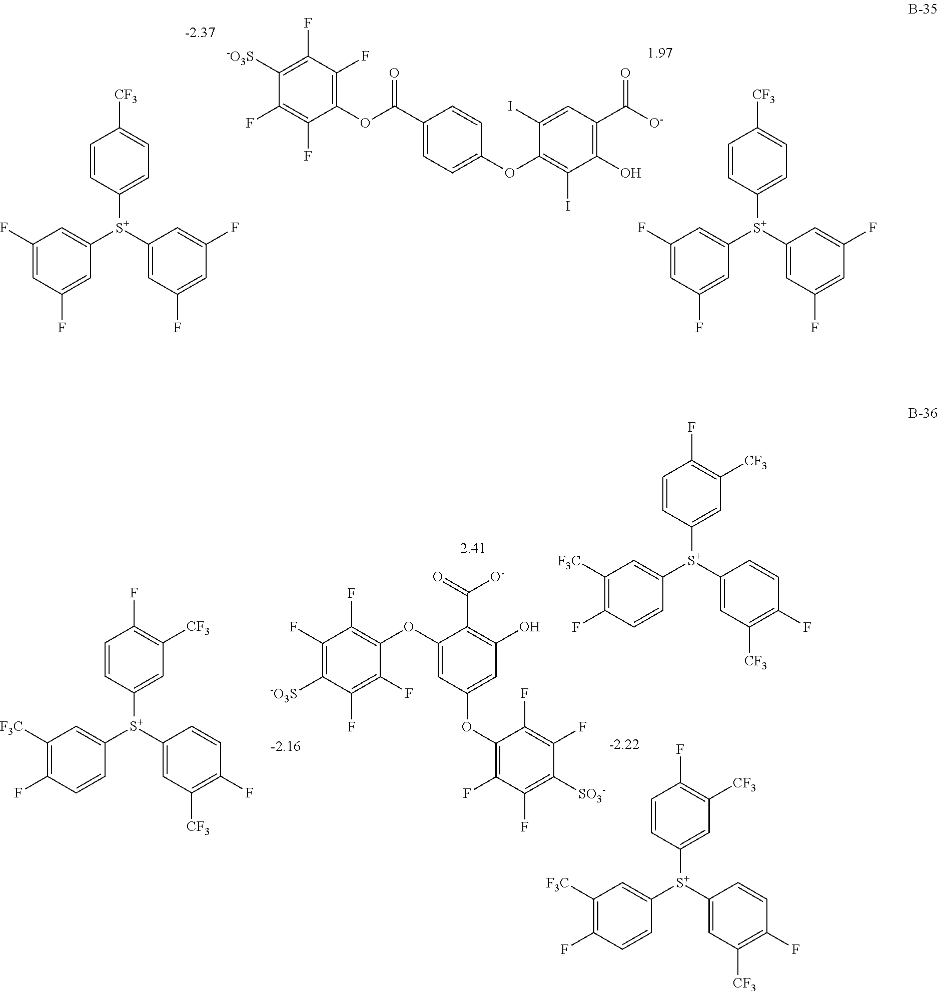

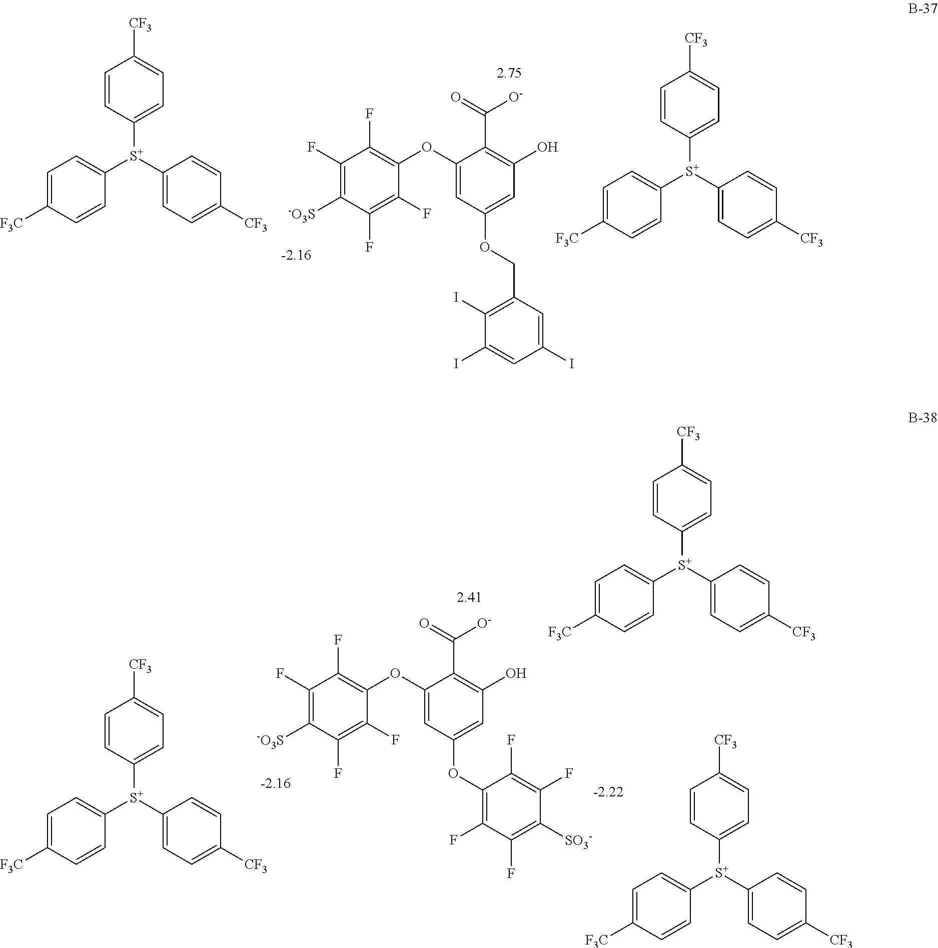

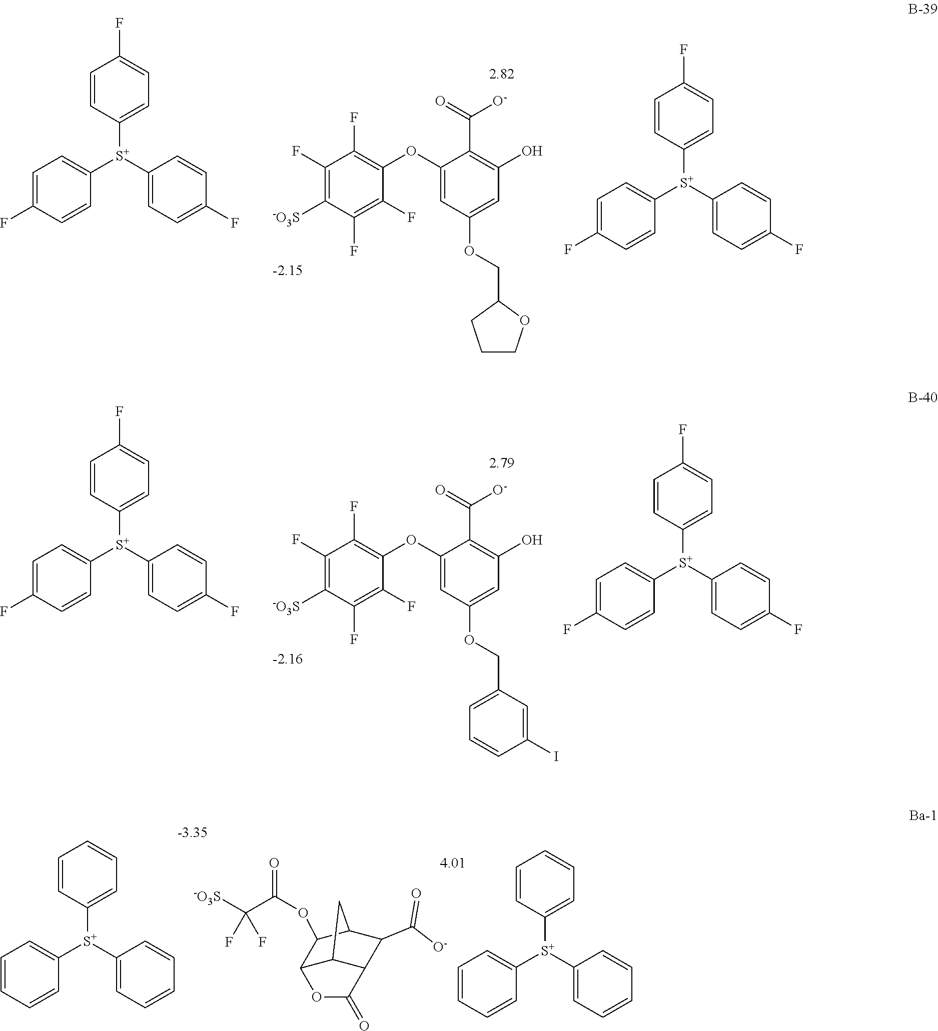

A synthesis example of the photoacid generator (B) will be described below.

### Synthesis Example 2: Synthesis of Compound B-1

### (Synthesis of Compound B-1-b)

B-1-a (225 g) was dissolved in acetone (475 g), and while the solution was stirred at 0° C. under a nitrogen stream, trifluoroacetic acid (275 g) and trifluoroacetic anhydride (460 g) were successively added dropwise thereto. After completion of the dropwise addition, stirring was performed at 25° C. for 20 hours. While a 10% aqueous sodium bicarbonate solution (8,585 g) and ethyl acetate (2,000 g) were stirred at 0° C., the above reaction solution was added dropwise thereto, the aqueous layer was then removed, and the organic layer was washed with a saturated aqueous sodium chloride solution and then concentrated. Ethyl acetate (158 g) and n-hexane (293 g) were added to the resulting residue, stirring was performed, and the obtained solid was collected by filtration. Ethyl acetate (200 g) was added to the solid, stirring was performed for one hour, and the obtained white powder was then collected by filtration and dried to obtain B-1-b (121 g).

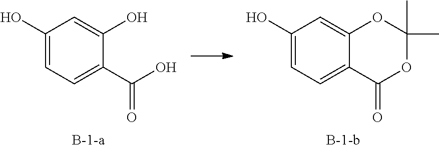

### (Synthesis of Compound B-1-d)

While cesium carbonate (106 g) and tetrahydrofuran (300 g) were stirred at −15° C. under a nitrogen stream, B-1-c (74 g) was added dropwise thereto, and a mixed liquid of B-1-b (30 g) and tetrahydrofuran (300 g) was then added dropwise thereto. After stirring for eight hours, the above reaction solution was added dropwise to a 1 mol/L aqueous hydrochloric acid solution (470 g), and n-hexane (200 mL) and ethyl acetate (200 mL) were added. After the aqueous layer was removed, the organic layer was washed with distilled water. After the organic layer was concentrated, column purification (hexane/ethyl acetate) was performed to obtain B-1-d (70 g).

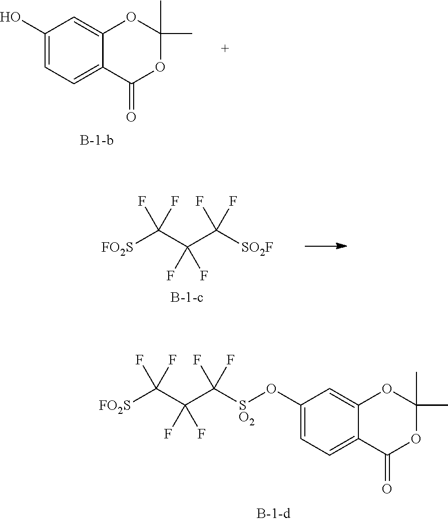

### (Synthesis of Compound B-1-e)

B-1-d (36 g), trifluoroacetic acid (180 g), and distilled water (18 g) were stirred at 55° C. for six hours, and distilled water (360 g) was then added at 20° C., followed by stirring for one hour. The obtained solid was collected by filtration, then washed with distilled water, and dried to obtain B-1-e (33 g).

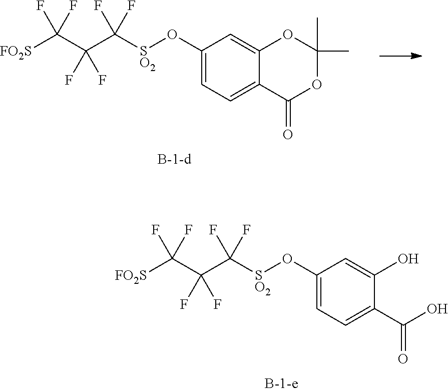

### (Synthesis of Compound B-1-f)

While B-1-e (37 g) and tetrahydrofuran (130 g) were stirred at 40° C., a liquid mixture of sodium bicarbonate (23 g) and distilled water (170 g) was added dropwise thereto, and stirring was performed for three hours. After the tetrahydrofuran was distilled off, the aqueous layer was washed twice with ethyl acetate (150 g). To the resulting aqueous solution, a 1 mol/L aqueous hydrochloric acid solution (142 g) was added dropwise at 0° C., and the aqueous layer was then washed twice with ethyl acetate (150 g). To the resulting aqueous solution, a 7% aqueous sodium bicarbonate solution (256 g) was added dropwise at room temperature, and stirring was performed to obtain an aqueous sodium bicarbonate solution of B-1-f (36 g).

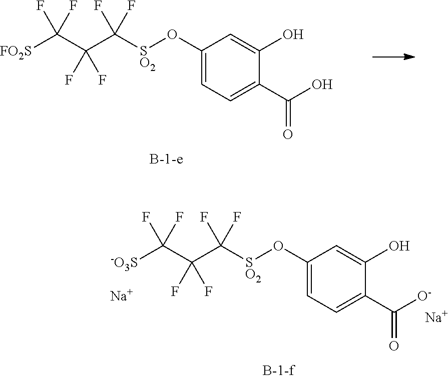

### (Synthesis of Compound B-1)

To the aqueous sodium bicarbonate solution of B-1-f (36 g) obtained as described above, methylene chloride (360 g) and B-1-g (66 g) were added, and the mixture was stirred at 25° C. for one hour. Ethyl acetate (100 g) was added to the resulting reaction liquid, and the mixture was left to stand. The aqueous layer was then removed, and the organic layer was washed with distilled water. The obtained organic layer was concentrated, diisopropyl ether (500 g) was then added thereto, and stirring was performed for one hour. The resulting white solid was collected by filtration and dried to obtain B-1 (62 g).

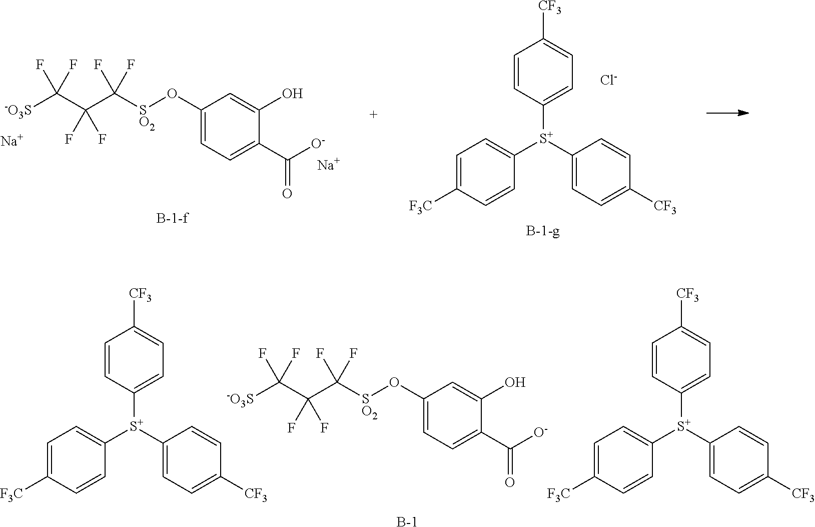

The obtained B-1 was identified by NMR (Nuclear Magnetic Resonance).

¹H NMR (400 MHz, (CD₃)₂CO) 6.45-6.57 (m, 2H) 7.79 (d, 1H), 8.15-8.45 (m, 24H)

Other compounds (B) used in Examples were also synthesized in the same manner as described above.

### [Photoacid Generator (C)]

The structures of photoacid generators (C) (compounds C-1 to C-47) shown in Table 3 are shown below. The photoacid generators (C) are compounds different from the photoacid generators (B).

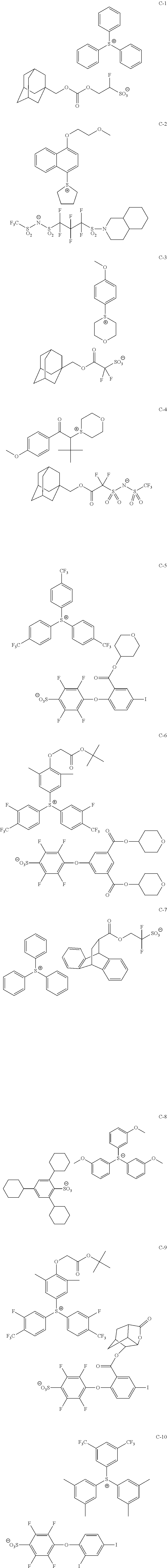

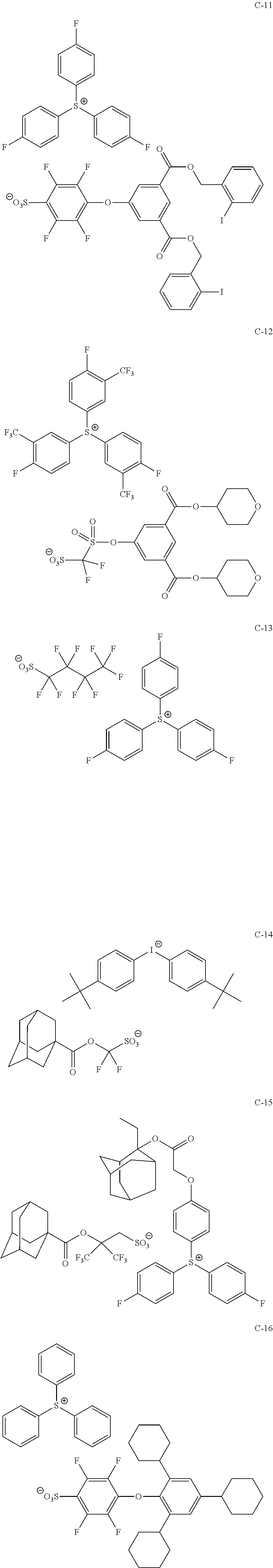

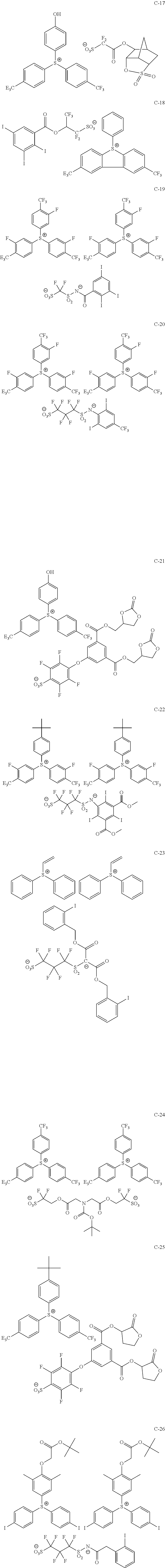

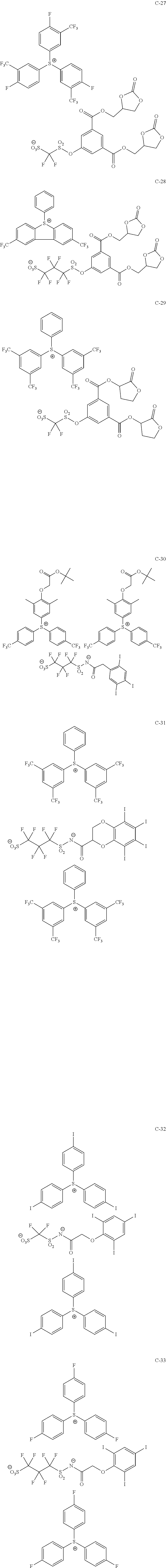

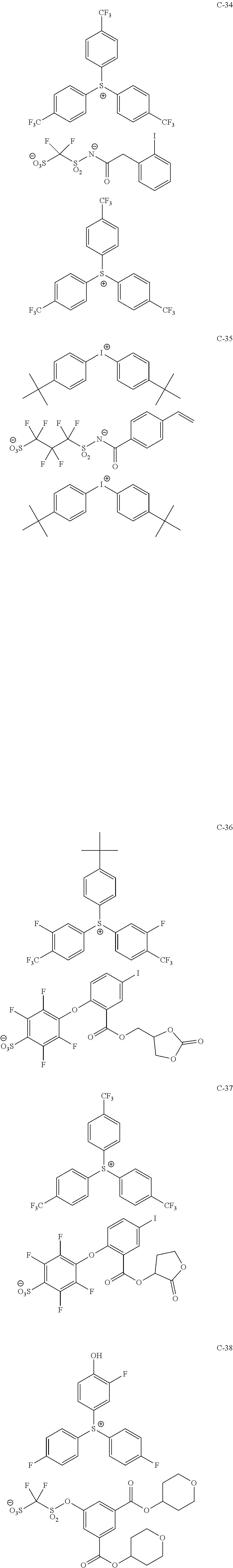

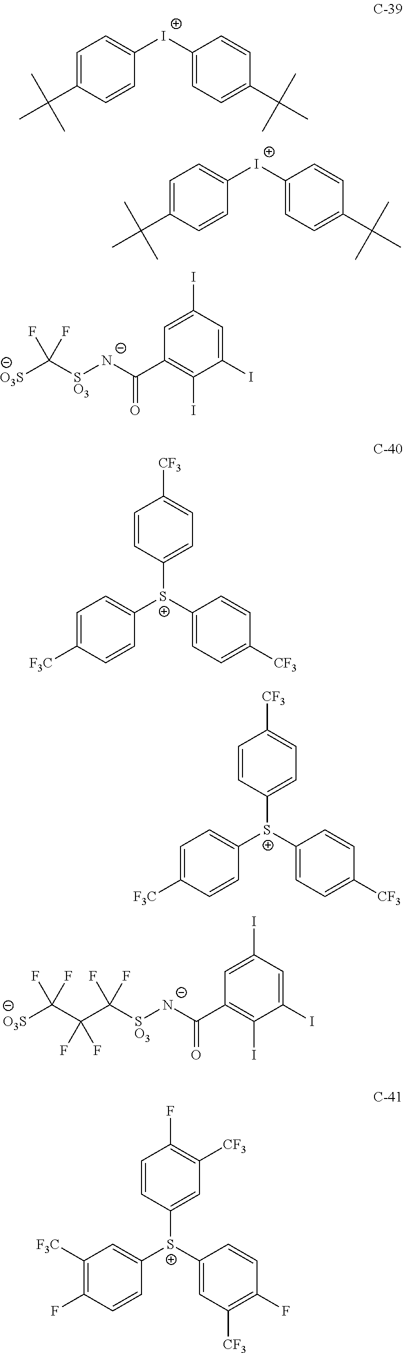

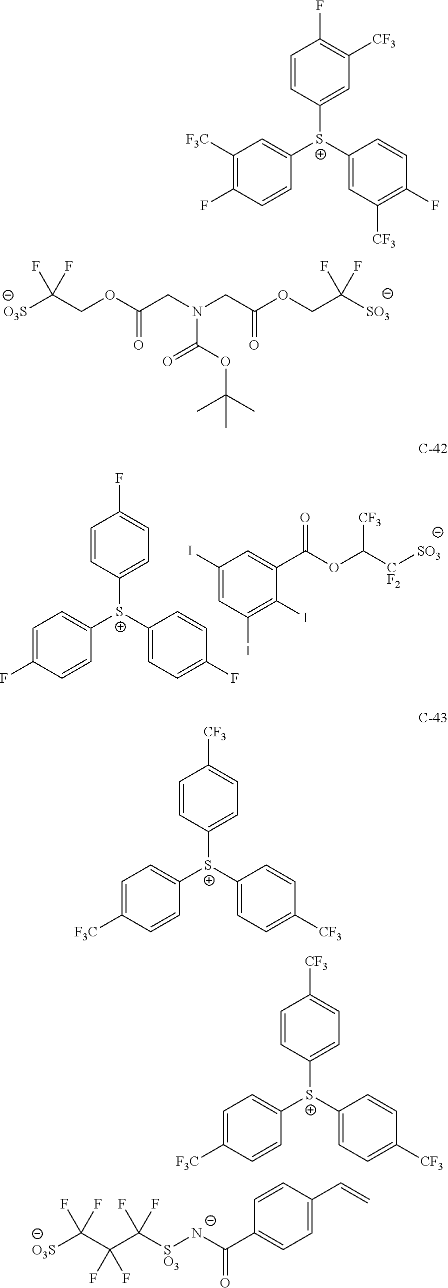

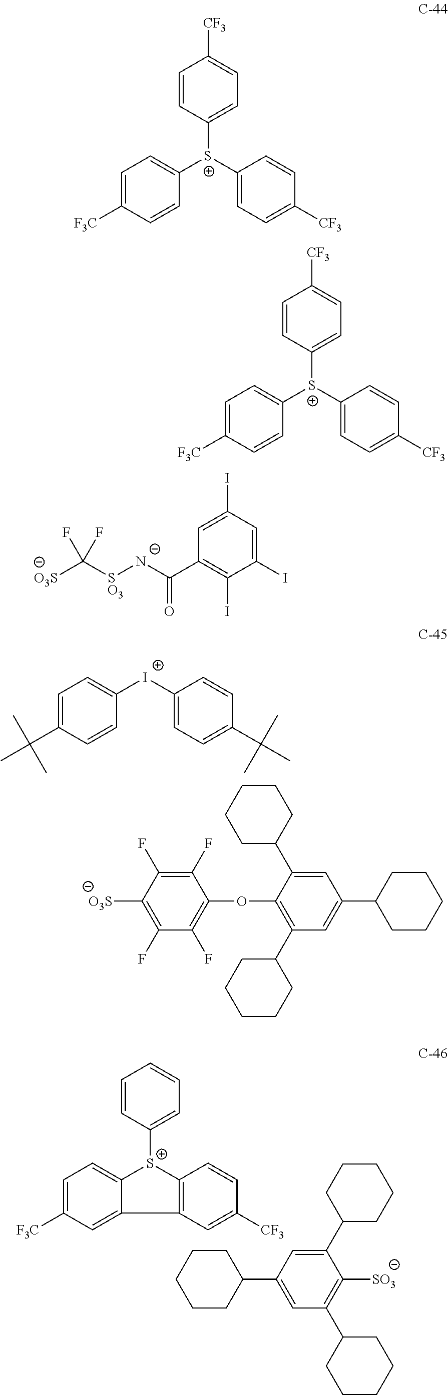

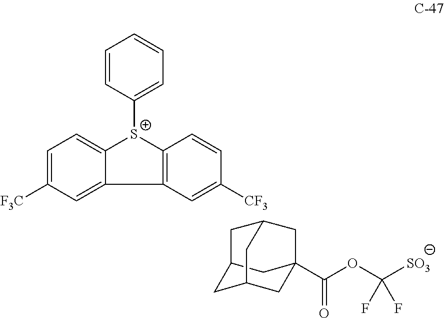

### [Photodegradable Quencher (D)]

The structures of photodegradable quenchers (D) (compounds D-1 to D-28) shown in Table 3 are shown below.

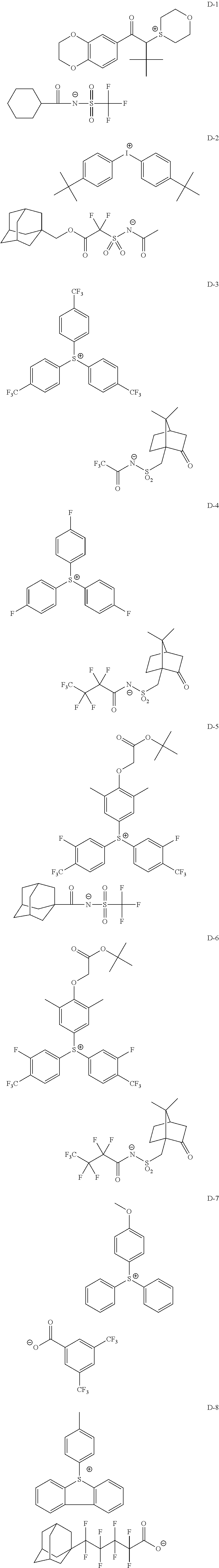

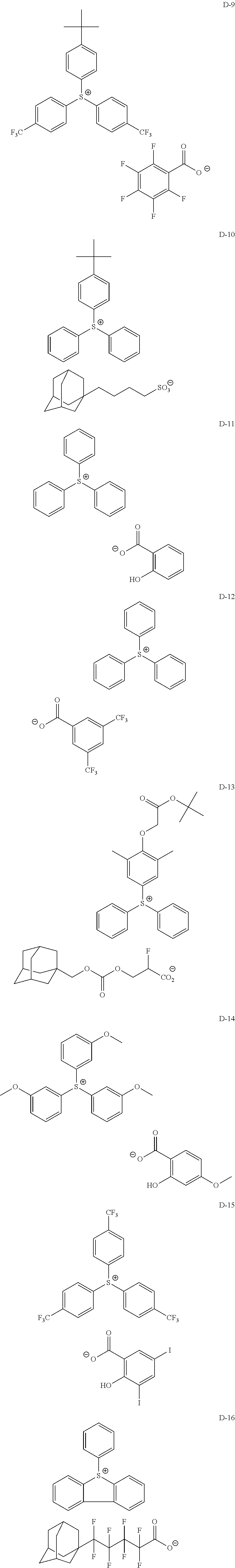

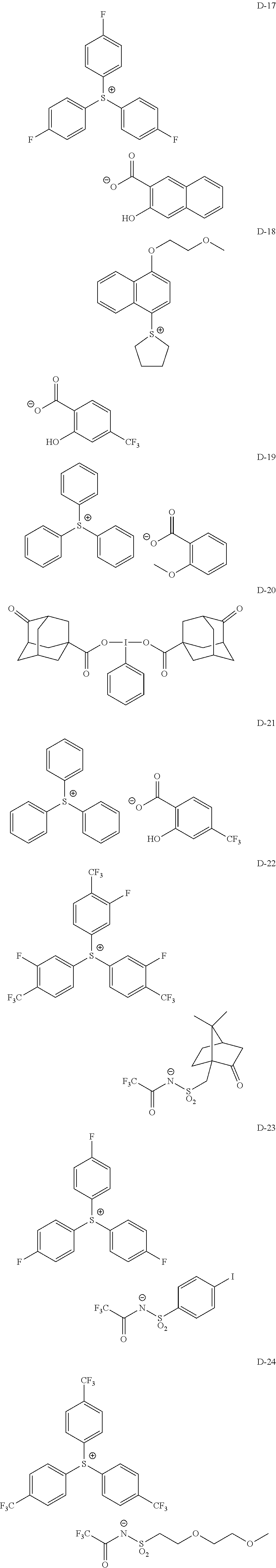

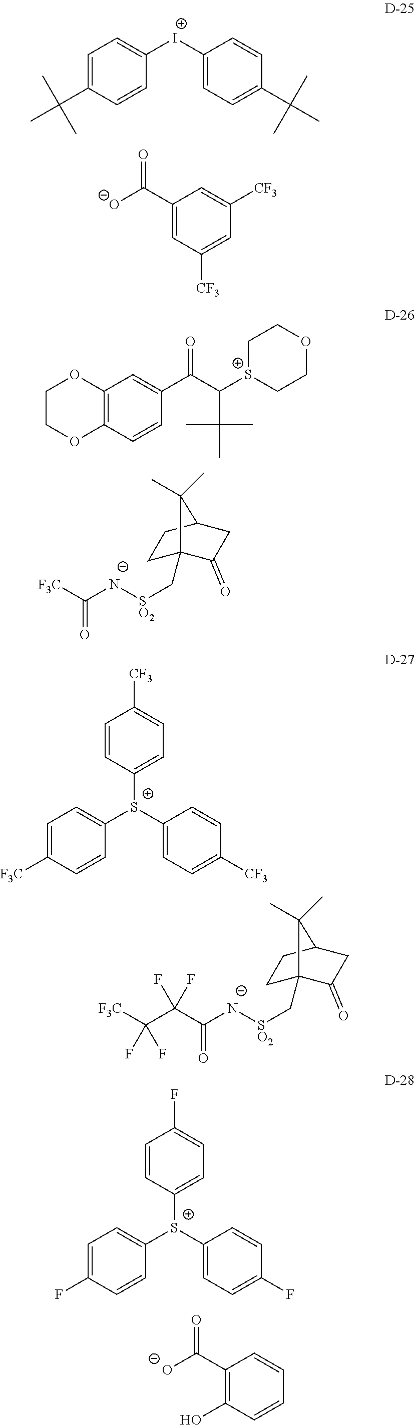

### [Non-Photodegradable Quencher (G)]

The structures of non-photodegradable quenchers (G) (compounds G-1 to G-5) shown in Table 3 are shown below.

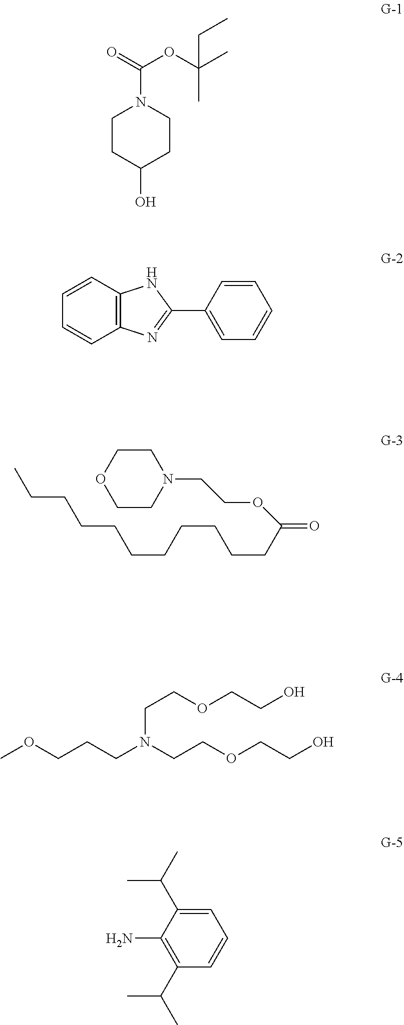 [SMILES: CCC(C)(C)OC(=O)N1CCC(O)CC1 ; c1ccc(-c2nc3ccccc3[nH]2)cc1 ; CCCCCCCCCCCC(=O)OCCN1CCOCC1 ; COCCCN(CCOCCO)CCOCCO ; CC(C)c1cccc(C(C)C)c1N]

### [Hydrophobic Resin (I)]

Hydrophobic resins I (resins I-1 to I-8) shown in Table 3 are described below.

The resins I-1 to I-8 used were synthesized in accordance with the above-described method for synthesizing the resin A-1 (Synthesis Example 1). Table 2 shows the compositional ratios of repeating units shown below (mass % ratio; corresponding to the order from the left), the weight-average molecular weight (Mw), and the dispersity (Mw/Mn).

The weight-average molecular weight (Mw) and the dispersity (Mw/Mn) of each of the resins I-1 to I-8 were (polystyrene-equivalent values) measured by GPC (carrier: tetrahydrofuran (THF)). The compositional ratios (mass % ratios) of the resins were measured by ¹³C-NMR (nuclear magnetic resonance).

TABLE 4

| | Mass ratio of repeating units (mass %) | | | | Mw | Mw/Mn |
|---|---|---|---|---|---|---|
| Resin I-1 | 50 | 45 | 5 | — | 6500 | 1.52 |
| Resin I-2 | 50 | 50 | — | — | 25000 | 1.65 |
| Resin I-3 | 30 | 65 | 5 | — | 22000 | 1.55 |
| Resin I-4 | 40 | 40 | 20 | — | 12000 | 1.68 |
| Resin I-5 | 40 | 50 | 5 | 5 | 5500 | 1.49 |
| Resin I-6 | 90 | 8 | 2 | — | 12000 | 1.63 |
| Resin I-7 | 20 | 30 | 40 | 10 | 13000 | 1.55 |
| Resin I-8 | 50 | 10 | 40 | — | 9000 | 1.51 |

The structural formulae of the resins I-1 to I-8 shown in Table 2 are shown below.

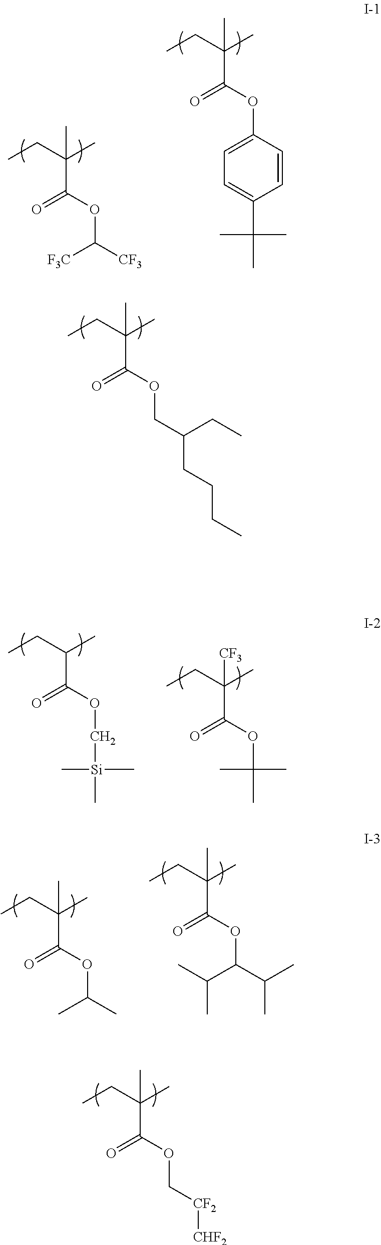

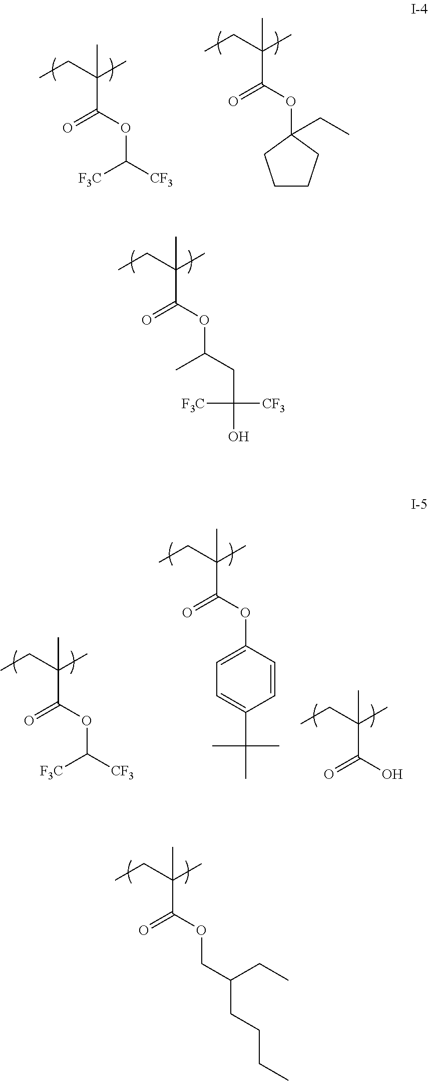

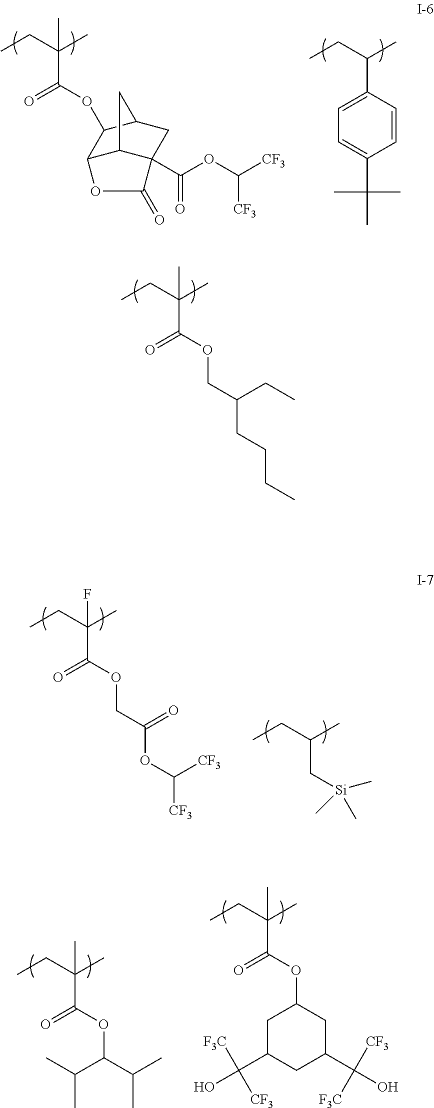

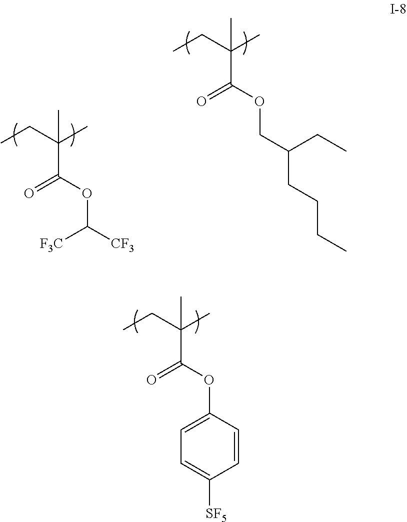

### [Surfactant]

Surfactants shown in Table 3 are as follows.
H-1: MVEGAFACE F176 (manufactured by DIC Corporation, fluorine-based surfactant)
H-2: MVEGAFACE R08 (manufactured by DIC Corporation, fluorine- and silicon-based surfactant)
H-3: PF656 (manufactured by OMNOVA Solutions, Inc., fluorine-based surfactant)

### [Solvent]

Solvents shown in Table 3 are as follows.
F-1: Propylene glycol monomethyl ether acetate (PGMEA)
F-2: Propylene glycol monomethyl ether (PGME)
F-3: Propylene glycol monoethyl ether (PGEE)
F-4: Cyclohexanone
F-5: Cyclopentanone
F-6: 2-Heptanone
F-7: Ethyl lactate
F-8: γ-Butyrolactone
F-9: Propylene carbonate

### [Preparation of Resist Composition]

The components shown in Table 3 were mixed such that the concentration of solid contents was 2.0 mass %. Next, the obtained liquid mixtures were each filtered through a polyethylene filter having a pore size of 50 nm, a nylon filter having a pore size of 10 nm, and a polyethylene filter having a pore size of 5 nm in this order to prepare resist compositions (Re-1 to Re-90 and CRe-1 and CRe-2).

Note that the solid contents mean all the components other than the solvent. The obtained resist compositions were used in Examples and Comparative Examples.

In the tables, the “content” columns indicate the content (mass %) of each component relative to the total solid contents in the resist composition.

TABLE 3

| | | | Photoacid | | Photoacid | | Photodegradable | | Non-photodegradable | | | | | | Solvent | |
|---|---|---|---|---|---|---|---|---|---|---|---|---|---|---|---|---|
| | Resin (A) | | generator (B) | | generator (C) | | quencher (D) | | quencher (G) | | Hydrophobic resin (I) | | Surfactant | | | Mixing |
| | | Content | | Content | | Content | | Content | | Content | | Content | | Content | | ratio |
| | Type | (mass %) | Type | (mass %) | Type | (mass %) | Type | (mass %) | Type | (mass %) | Type | (mass %) | Type | (mass %) | Type | (mass ratio) |
| Re-1 | A-1 | 62.0 | B-1 | 32.0 | C-8 | 3.0 | D-21 | 3.0 | — | — | — | — | — | — | F-1/F-2 | 80/20 |
| Re-2 | A-2 | 58.0 | B-2 | 38.0 | C-1 | 2.0 | D-1 | 2.0 | — | — | — | — | — | — | F-1/F-5 | 85/15 |
| Re-3 | A-3 | 48.0 | B-3 | 44.0 | C-2 | 4.0 | D-2 | 4.0 | — | — | — | — | — | — | F-1/F-2 | 80/20 |
| Re-4 | A-4 | 50.0 | B-4 | 40.0 | C-3 | 5.0 | D-3 | 5.0 | — | — | — | — | — | — | F-1/F-2/F-8 | 40/20/40 |
| Re-5 | A-5 | 58.0 | B-5 | 38.0 | C-4 | 2.0 | D-4 | 2.0 | — | — | — | — | — | — | F-1/F-2 | 80/20 |
| Re-6 | A-6 | 65.0 | B-6 | 23.0 | C-5 | 6.0 | D-5 | 6.0 | — | — | — | — | — | — | F-1/F-9 | 85/15 |
| Re-7 | A-7 | 42.0 | B-7 | 33.9 | C-6 | 12.0 | D-6 | 12.0 | — | — | — | — | H-1 | 0.1 | F-1/F-7 | 90/10 |
| Re-8 | A-8 | 31.0 | B-8 | 55.0 | C-7 | 7.0 | D-7 | 7.0 | — | — | — | — | — | — | F-1/F-2 | 80/20 |
| Re-9 | A-9 | 50.0 | B-9 | 35.0 | C-8 | 8.0 | D-8 | 7.0 | — | — | — | — | — | — | F-1/F-2 | 80/20 |
| Re-10 | A-10 | 50.0 | B-10 | 40.0 | C-9 | 5.0 | D-9 | 5.0 | — | — | — | — | — | — | F-1/F-2/F-6 | 70/20/10 |
| Re-11 | A-11 | 55.0 | B-11 | 30.0 | C-10 | 8.0 | D-10 | 7.0 | — | — | — | — | — | — | F-1/F-2 | 80/20 |
| Re-12 | A-12 | 90.0 | B-12 | 6.0 | C-11 | 2.0 | D-11 | 2.0 | — | — | — | — | — | — | F-1/F-4 | 80/20 |
| Re-13 | A-13 | 52.0 | B-13 | 30.0 | C-12 | 8.0 | D-12 | 8.0 | — | — | I-1 | 2.0 | — | — | F-1/F-2 | 80/20 |
| Re-14 | A-14 | 63.0 | B-14 | 20.0 | C-13 | 7.0 | D-13 | 7.0 | — | — | I-2 | 2.9 | H-2 | 0.1 | F-4 | 100 |
| Re-15 | A-15 | 65.0 | B-15 | 24.0 | C-14 | 4.0 | D-14 | 4.0 | G-1 | 3.0 | — | — | — | — | F-1/F-2/F-8 | 85/12/3 |
| Re-16 | A-16 | 80.0 | B-16 | 11.0 | C-15 | 3.0 | D-15 | 1.0 | G-2 | 5.0 | — | — | — | — | F-1/F-5 | 80/20 |
| Re-17 | A-17 | 63.5 | B-17 | 24.0 | C-16 | 6.5 | D-16 | 2.0 | — | — | I-3 | 4.0 | — | — | F-1/F-2 | 75/25 |
| Re-18 | A-18 | 87.0 | B-18 | 8.0 | C-17 | 2.0 | D-17 | 1.0 | G-3 | 2.0 | — | — | — | — | F-1/F-2/F-8 | 34/33/33 |
| Re-19 | A-19 | 65.5 | B-19 | 20.0 | C-18 | 6.5 | D-18 | 6.0 | — | — | I-4 | 2.0 | — | — | F-1/F-2 | 80/20 |
| Re-20 | A-20 | 68.0 | B-20 | 23.0 | C-19 | 2.0 | D-19 | 2.0 | G-4 | 5.0 | — | — | — | — | F-1/F-9 | 80/20 |
| Re-21 | A-21 | 55.0 | B-21 | 37.0 | C-20 | 4.0 | D-20 | 2.0 | G-5 | 2.0 | — | — | — | — | F-1/F-2/F-8 | 85/12/3 |
| Re-22 | A-22 | 53.0 | B-22 | 30.0 | C-21 | 4.0 | D-21 | 4.0 | — | — | I-5 | 8.9 | H-3 | 0.1 | F-1/F-2 | 85/15 |
| Re-23 | A-23 | 80.0 | B-23 | 12.0 | C-22 | 5.0 | — | — | — | — | I-6 | 3.0 | — | — | F-1/F-2 | 80/20 |
| Re-24 | A-24 | 82.0 | B-24 | 8.0 | C-23 | 2.0 | — | — | G-1 | 3.0 | I-7 | 5.0 | — | — | F-1/F-2/F-6 | 80/15/5 |
| Re-25 | A-25 | 70.0 | B-25 | 18.0 | C-24 | 8.0 | — | — | — | — | I-8 | 4.0 | — | — | F-1/F-2 | 90/10 |
| Re-26 | A-26 | 65.0 | B-26 | 25.0 | C-25 | 10.0 | — | — | — | — | — | — | — | — | F-1/F-4 | 80/20 |
| Re-27 | A-27 | 55.0 | B-27 | 27.0 | C-26 | 15.0 | D-22 | 3.0 | — | — | — | — | — | — | F-1/F-2 | 80/20 |
| Re-28 | A-28 | 45.0 | B-28 | 35.0 | C-27 | 15.9 | D-23 | 4.0 | — | — | — | — | H-3 | 0.1 | F-4 | 100 |
| Re-29 | A-29 | 40.0 | B-29 | 45.0 | C-28 | 5.0 | D-24 | 1.1 | — | — | I-5 | 8.9 | — | — | F-1/F-3 | 85/15 |
| Re-30 | A-30 | 75.0 | B-30 | 12.0 | C-29 | 5.0 | D-11 | 5.0 | — | — | I-6 | 3.0 | — | — | F-1/F-5 | 80/20 |
| Re-31 | A-31 | 60.1 | B-31 | 15.0 | C-30 | 9.9 | D-12 | 9.9 | — | — | I-7 | 5.0 | H-1 | 0.1 | F-1/F-2 | 75/25 |
| Re-32 | A-32 | 61.0 | B-32 | 15.0 | C-1 | 10.0 | D-13 | 10.0 | — | — | I-8 | 4.0 | — | — | F-1/F-2/F-8 | 40/20/40 |
| Re-33 | A-33 | 70.0 | B-33 | 25.0 | C-2 | 5.0 | — | — | — | — | — | — | — | — | F-1/F-2 | 80/20 |
| Re-34 | A-34 | 75.0 | B-34 | 22.0 | — | — | — | — | G-1 | 3.0 | — | — | — | — | F-1/F-9 | 80/20 |
| Re-35 | A-35 | 60.0 | B-35 | 40.0 | — | — | — | — | — | — | — | — | — | — | F-1/F-7 | 80/20 |
| Re-36 | A-1 | 65.0 | B-36 | 31.0 | — | — | D-7 | 2.0 | — | — | I-1 | 2.0 | — | — | F-1/F-2 | 85/15 |
| Re-37 | A-2 | 67.0 | B-4 | 30.0 | — | — | D-1 | 3.0 | — | — | — | — | — | — | F-1/F-2 | 90/10 |
| Re-38 | A-3 | 50.0 | B-3 | 40.0 | C-22 | 5.0 | D-2 | 5.0 | — | — | — | — | — | — | F-1/F-2/F-6 | 60/20/20 |
| Re-39 | A-4 | 50.0 | B-2 | 40.0 | C-23 | 5.0 | D-3 | 5.0 | — | — | — | — | — | — | F-1/F-2 | 90/10 |
| Re-40 | A-5 | 48.0 | B-1 | 44.0 | C-24 | 4.0 | D-4 | 4.0 | — | — | — | — | — | — | F-1/F-2 | 80/20 |
| Re-41 | A-6 | 60.0 | B-20 | 27.0 | C-25 | 7.0 | D-5 | 3.0 | — | — | I-6 | 3.0 | — | — | F-1/F-2 | 80/20 |
| Re-42 | A-6 | 60.0 | B-3 | 14.0 | C-25 | 5.0 | D-5 | 5.0 | — | — | I-6 | 3.0 | — | — | F-1/F-2 | 80/20 |
| | | | B-4 | 13.0 | | | | | — | — | | | — | — | | |
| Re-43 | A-6 | 60.0 | B-21 | 14.0 | C-25 | 5.0 | D-5 | 5.0 | — | — | I-6 | 3.0 | — | — | F-1/F-2 | 80/20 |
| | | | B-36 | 13.0 | | | | | | | | | | | | |
| Re-44 | A-6 | 60.0 | B-3 | 14.0 | C-25 | 5.0 | D-5 | 5.0 | — | — | I-6 | 3.0 | — | — | F-1/F-2 | 80/20 |
| | | | Ba-1 | 13.0 | | | | | | | | | | | | |
| Re-45 | A-6 | 50.0 | B-20 | 27.0 | C-25 | 5.0 | D-5 | 10.0 | — | — | I-6 | 3.0 | — | — | F-1/F-2 | 80/20 |
| | | | | | C-21 | 5.0 | | | | | | | | | | |
| Re-46 | A-6 | 50.0 | B-20 | 27.0 | C-25 | 5.0 | D-5 | 10.0 | — | — | I-6 | 3.0 | — | — | F-1/F-2 | 80/20 |
| | | | | | C-23 | 5.0 | | | | | | | | | | |
| Re-47 | A-6 | 50.0 | B-20 | 27.0 | C-25 | 10.0 | D-3 | 5.0 | — | — | I-6 | 3.0 | — | — | F-1/F-2 | 80/20 |
| | | | | | | | D-11 | 5.0 | — | — | — | — | — | — | | |
| CRe-1 | A-1 | 65.0 | Ba-1 | 29.0 | C-8 | 3.0 | D-21 | 3.0 | — | — | — | — | — | — | F-1/F-2 | 80/20 |
| CRe-2 | A-1 | 65.0 | — | — | C-8 | 25.0 | D-15 | 10.0 | — | — | — | — | — | — | F-1/F-2 | 80/20 |
| Re-48 | A-36 | 60.0 | B-1 | 29.0 | C-31 | 9.0 | D-1 | 2.0 | — | — | — | — | — | — | F-1/F-7 | 90/10 |
| Re-49 | A-37 | 57.0 | B-2 | 31.0 | C-32 | 9.0 | D-2 | 3.0 | — | — | — | — | — | — | F-1/F-2 | 80/20 |
| Re-50 | A-38 | 57.0 | B-3 | 25.0 | C-33 | 10.0 | D-3 | 2.8 | G-5 | 1.0 | I-3 | 4.0 | H-1 | 0.2 | F-1/F-2 | 80/20 |
| Re-51 | A-39 | 50.0 | B-4 | 40.0 | C-34 | 8.0 | D-4 | 2.0 | — | — | — | — | — | — | F-1/F-2/F-6 | 70/20/10 |
| Re-52 | A-13 | 52.0 | B-5 | 35.0 | C-35 | 5.0 | D-5 | 6.0 | — | — | I-4 | 2.0 | — | — | F-1/F-2 | 80/20 |
| Re-53 | A-14 | 63.0 | B-6 | 20.0 | C-36 | 8.0 | D-6 | 6.0 | G-1 | 3.0 | — | — | — | — | F-1/F-4 | 80/20 |
| Re-54 | A-15 | 64.0 | B-7 | 32.0 | C-37 | 2.0 | D-7 | 2.0 | — | — | — | — | — | — | F-1/F-2 | 80/20 |
| Re-55 | A-16 | 60.0 | B-8 | 30.0 | C-38 | 8.0 | D-1 | 2.0 | — | — | — | — | — | — | F-4 | 100 |
| Re-56 | A-40 | 57.0 | B-29 | 28.0 | C-28 | 5.0 | D-24 | 1.1 | — | — | I-5 | 8.9 | — | — | F-1/F-3 | 85/15 |
| Re-57 | A-41 | 51.0 | B-19 | 25.0 | C-35 | 5.0 | D-25 | 5.0 | G-1 | 3.0 | — | — | — | — | F-1/F-2/F-8 | 85/12/3 |
| | | | | | C-39 | 11.0 | | | | | | | | | | |
| Re-58 | A-42 | 47.0 | B-8 | 15.0 | C-21 | 15.0 | D-24 | 8.0 | — | — | — | — | — | — | F-1/F-2/F-8 | 85/12/3 |
| | | | | | C-40 | 15.0 | | | | | | | | | | |
| Re-59 | A-43 | 54.0 | B-20 | 21.0 | C-12 | 12.0 | D-22 | 2.0 | — | — | — | — | — | — | F-1/F-2/F-8 | 85/12/3 |
| | | | | | C-41 | 11.0 | | | | | | | | | | |
| Re-60 | A-44 | 67.0 | B-4 | 8.0 | C-21 | 8.0 | D-27 | 4.0 | — | — | — | — | — | — | F-1/F-2/F-8 | 85/12/3 |
| | | | | | C-40 | 13.0 | | | | | | | | | | |
| Re-61 | A-45 | 62.0 | B-1 | 15.0 | C-33 | 5.0 | D-28 | 11.0 | — | — | — | — | — | — | F-1/F-2/F-6 | 70/20/10 |
| | | | | | C-42 | 7.0 | | | | | | | | | | |
| Re-62 | A-46 | 58.0 | B-2 | 17.0 | C-14 | 10.0 | D-2 | 3.0 | — | — | — | — | — | — | F-1/F-2/F-8 | 85/12/3 |
| | | | | | C-39 | 12.0 | | | | | | | | | | |
| Re-63 | A-47 | 69.0 | B-3 | 14.0 | C-37 | 7.0 | D-3 | 2.0 | — | — | — | — | — | — | F-1/F-2/F-8 | 85/12/3 |
| | | | | | C-43 | 8.0 | | | | | | | | | | |
| Re-64 | A-48 | 60.0 | B-29 | 21.0 | C-35 | 7.0 | D-25 | 5.0 | — | — | — | — | — | — | F-1/F-2/F-8 | 85/12/3 |
| | | | | | C-39 | 7.0 | | | | | | | | | | |
| Re-65 | A-49 | 65.0 | B-3 | 16.0 | C-39 | 7.0 | D-25 | 5.0 | — | — | — | — | — | — | F-1/F-2/F-6 | 70/20/10 |
| | | | | | C-45 | 7.0 | | | | | | | | | | |
| Re-66 | A-50 | 57.0 | B-20 | 15.0 | C-35 | 5.0 | D-25 | 5.0 | — | — | — | — | — | — | F-1/F-2/F-8 | 85/12/3 |
| | | | | | C-45 | 18.0 | | | | | | | | | | |
| Re-67 | A-41 | 63.0 | B-15 | 22.0 | C-35 | 5.0 | D-2 | 5.0 | — | — | — | — | — | — | F-1/F-2/F-6 | 70/20/10 |
| | | | | | C-39 | 5.0 | | | | | | | | | | |
| Re-68 | A-42 | 62.0 | B-20 | 10.0 | C-21 | 11.0 | D-27 | 5.0 | — | — | — | — | — | — | F-1/F-2/F-8 | 85/12/3 |
| | | | | | C-40 | 12.0 | | | | | | | | | | |
| Re-69 | A-39 | 46.0 | B-4 | 18.0 | C-21 | 13.0 | D-27 | 10.0 | — | — | — | — | — | — | F-1/F-2/F-8 | 85/12/3 |
| | | | | | C-43 | 13.0 | | | | | | | | | | |
| Re-70 | A-40 | 61.0 | B-20 | 11.0 | C-6 | 5.0 | D-28 | 11.0 | — | — | — | — | — | — | F-1/F-2/F-8 | 85/12/3 |
| | | | | | C-19 | 12.0 | | | | | | | | | | |
| Re-71 | A-6 | 55.0 | B-1 | 12.0 | C-33 | 7.0 | D-4 | 3.8 | G-5 | 2.0 | I-6 | 4.0 | H-1 | 0.2 | F-1/F-2/F-8 | 85/12/3 |
| | | | | | C-42 | 16.0 | | | | | | | | | | |
| Re-72 | A-36 | 60.0 | B-20 | 20.0 | C-20 | 4.0 | D-27 | 8.0 | — | — | — | — | — | — | F-1/F-2/F-8 | 85/12/3 |
| | | | | | C-38 | 8.0 | | | | | | | | | | |
| Re-73 | A-5 | 56.0 | B-11 | 19.0 | C-21 | 6.0 | D-27 | 4.0 | — | — | — | — | — | — | F-1/F-2/F-8 | 85/12/3 |
| | | | | | C-40 | 15.0 | | | | | | | | | | |
| Re-74 | A-14 | 55.0 | B-6 | 25.0 | C-37 | 2.0 | D-27 | 8.0 | — | — | — | — | — | — | F-1/F-2 | 80/20 |
| | | | | | C-43 | 10.0 | | | | | | | | | | |
| Re-75 | A-39 | 62.0 | B-7 | 12.0 | C-21 | 8.0 | D-27 | 3.0 | — | — | — | — | — | — | F-1/F-2/F-8 | 85/12/3 |
| | | | | | C-44 | 15.0 | | | | | | | | | | |
| Re-76 | A-28 | 54.0 | B-8 | 30.0 | C-35 | 5.0 | D-2 | 6.0 | — | — | — | — | — | — | F-1/F-2/F-8 | 85/12/3 |
| | | | | | C-39 | 5.0 | | | | | | | | | | |
| Re-77 | A-6 | 54.0 | B-11 | 15.0 | C-12 | 20.0 | D-22 | 6.0 | — | — | — | — | — | — | F-1/F-2 | 80/20 |
| | | | | | C-41 | 5.0 | | | | | | | | | | |
| Re-78 | A-43 | 61.0 | B-20 | 15.0 | C-11 | 10.0 | D-28 | 2.0 | — | — | — | — | — | — | F-1/F-2/F-6 | 70/20/10 |
| | | | | | C-42 | 12.0 | | | | | | | | | | |
| Re-79 | A-6 | 67.0 | B-8 | 11.0 | C-33 | 6.0 | D-22 | 5.0 | — | — | — | — | — | — | F-1/F-2/F-8 | 85/12/3 |
| | | | | | C-46 | 11.0 | | | | | | | | | | |
| Re-80 | A-28 | 69.0 | B-7 | 8.0 | C-31 | 9.0 | D-3 | 6.0 | — | — | — | — | — | — | F-1/F-2/F-8 | 85/12/3 |
| | | | | | C-47 | 8.0 | | | | | | | | | | |
| Re-81 | A-47 | 71.0 | B-1 | 15.0 | C-22 | 5.0 | D-26 | 4.0 | — | — | — | — | — | — | F-1/F-7 | 90/10 |
| | | | | | C-25 | 5.0 | | | | | | | | | | |
| Re-82 | A-46 | 60.0 | B-1 | 7.0 | C-12 | 10.0 | D-26 | 2.0 | — | — | — | — | — | — | F-1/F-2 | 80/20 |
| | | | | | C-19 | 21.0 | | | | | | | | | | |
| Re-83 | A-49 | 58.0 | B-11 | 13.0 | C-24 | 25.0 | D-26 | 4.0 | — | — | — | — | — | — | F-1/F-2/F-8 | 85/12/3 |
| Re-84 | A-49 | 56.0 | B-2 | 17.0 | C-24 | 22.0 | D-28 | 5.0 | — | — | — | — | — | — | F-1/F-2/F-8 | 85/12/3 |
| Re-85 | A-50 | 53.0 | B-20 | 19.0 | C-6 | 5.0 | D-28 | 11.0 | — | — | — | — | — | — | F-1/F-2/F-8 | 85/12/3 |
| | | | | | C-19 | 12.0 | | | | | | | | | | |
| Re-86 | A-41 | 59.0 | B-4 | 10.0 | C-14 | 10.0 | D-25 | 9.0 | — | — | — | — | — | — | F-1/F-2/F-8 | 85/12/3 |
| | | | | | C-39 | 12.0 | | | | | | | | | | |
| Re-87 | A-42 | 56.0 | B-37 | 31.0 | C-37 | 9.0 | D-27 | 4.0 | — | — | — | — | — | — | F-1/F-2/F-8 | 85/12/3 |
| Re-88 | A-41 | 60.0 | B-38 | 28.0 | C-36 | 9.0 | D-26 | 3.0 | — | — | — | — | — | — | F-1/F-2/F-8 | 85/12/3 |
| Re-89 | A-40 | 65.0 | B-39 | 26.0 | C-35 | 7.0 | D-25 | 2.0 | — | — | — | — | — | — | F-1/F-2/F-8 | 85/12/3 |
| Re-90 | A-39 | 51.0 | B-40 | 35.0 | C-34 | 10.0 | D-24 | 4.0 | — | — | — | — | — | — | F-1/F-2/F-8 | 85/12/3 |

### [Pattern Formation]

### [EUV Exposure, Organic Solvent Development]

An underlayer film-forming composition AL412 (manufactured by Brewer Science, Inc.) was applied to a silicon wafer having a diameter of 12 inches and baked at 205° C. for 60 seconds to form an underlying film having a film thickness of 20 nm. A resist composition shown in Table 4 was applied to the underlying film and baked at 100° C. for 60 seconds to form a resist film having a film thickness of 30 nm.

The resulting silicon wafer having the resist film was subjected to pattern irradiation using an EUV exposure apparatus (Micro Exposure Tool, manufactured by Exitech Ltd., NA 0.3, Quadrupole, outer sigma 0.68, inner sigma 0.36) such that the average line width of the pattern was 14 nm. As a reticle, a mask having a line size of 14 nm and a line:space ratio of 1:1 was used.

The resist film after exposure was baked at 90° C. for 60 seconds, then developed with n-butyl acetate for 30 seconds, and spin-dried to obtain a negative-type pattern.

### [Evaluation]

### <Defect Evaluation (Defect Suppression Performance)>

The pattern obtained by the above-described method was evaluated in accordance with the following evaluation criteria by counting the number of defects per silicon wafer using UVision5 (manufactured by AMAT) and SEMVisionG4 (manufactured by AMAT). The smaller the number of defects, the better the defect suppression performance, and a rating of “E” or higher can be regarded as a pass.
“A”: The number of defects is 50 or less.
“B”: The number of defects is more than 50 and 100 or less.
“C”: The number of defects is more than 100 and 200 or less.
“D”: The number of defects is more than 200 and 300 or less.
“E”: The number of defects is more than 300 and 500 or less.
“F”: The number of defects is more than 500.

### <Roughness Performance>

The roughness performance was evaluated by line width roughness (LWR) performance.

The pattern obtained by the above-described method was observed from above the pattern using a critical dimension scanning electron microscope (SEM (S-9380II, Hitachi, Ltd.)). The line width of the pattern was observed at 250 points, and the standard deviation (a) thereof was determined. The measurement variation of the line width was evaluated on the basis of 36, and the value of 3σ was defined as LWR (nm). The smaller the value of LWR, the better the LWR performance.

The LWR performance (nm) is preferably 3.0 nm or less, more preferably 2.8 nm or less, still more preferably 2.6 nm or less, still further more preferably 2.4 nm or less, particularly preferably 2.2 nm or less, most preferably 2.0 nm or less.

The evaluation results are shown in the following tables.

TABLE 4

| | | LWR | |
|---|---|---|---|
| | Resist | performance | Defect suppression |
| | composition | (nm) | performance |
| Example 1-1 | Re-1 | 2.01 | B |
| Example 1-2 | Re-2 | 2.02 | B |
| Example 1-3 | Re-3 | 2.22 | C |
| Example 1-4 | Re-4 | 2.24 | C |
| Example 1-5 | Re-5 | 1.82 | A |
| Example 1-6 | Re-6 | 2.30 | C |
| Example 1-7 | Re-7 | 1.85 | A |
| Example 1-8 | Re-8 | 1.90 | A |
| Example 1-9 | Re-9 | 1.92 | A |
| Example 1-10 | Re-10 | 2.32 | C |
| Example 1-11 | Re-11 | 2.33 | C |
| Example 1-12 | Re-12 | 2.04 | B |
| Example 1-13 | Re-13 | 2.12 | B |
| Example 1-14 | Re-14 | 2.25 | C |
| Example 1-15 | Re-15 | 1.82 | A |
| Example 1-16 | Re-16 | 2.01 | B |
| Example 1-17 | Re-17 | 2.01 | B |
| Example 1-18 | Re-18 | 2.28 | C |
| Example 1-19 | Re-19 | 1.95 | A |
| Example 1-20 | Re-20 | 2.14 | B |
| Example 1-21 | Re-21 | 1.82 | A |
| Example 1-22 | Re-22 | 2.16 | B |
| Example 1-23 | Re-23 | 2.10 | B |
| Example 1-24 | Re-24 | 1.86 | A |
| Example 1-25 | Re-25 | 1.83 | A |
| Example 1-26 | Re-26 | 2.38 | C |
| Example 1-27 | Re-27 | 2.12 | B |
| Example 1-28 | Re-28 | 2.41 | D |
| Example 1-29 | Re-29 | 1.84 | A |
| Example 1-30 | Re-30 | 2.09 | B |
| Example 1-31 | Re-31 | 2.25 | C |
| Example 1-32 | Re-32 | 2.61 | E |
| Example 1-33 | Re-33 | 2.45 | D |
| Example 1-34 | Re-34 | 2.16 | B |
| Example 1-35 | Re-35 | 1.87 | A |
| Example 1-36 | Re-36 | 2.04 | B |
| Example 1-37 | Re-37 | 2.36 | C |
| Example 1-38 | Re-38 | 2.25 | C |
| Example 1-39 | Re-39 | 2.11 | B |
| Example 1-40 | Re-40 | 1.93 | A |
| Example 1-41 | Re-41 | 2.18 | B |
| Example 1-42 | Re-42 | 2.29 | C |
| Example 1-43 | Re-43 | 2.12 | B |
| Example 1-44 | Re-44 | 2.30 | C |
| Example 1-45 | Re-45 | 2.13 | B |
| Example 1-46 | Re-46 | 2.14 | B |
| Example 1-47 | Re-47 | 2.12 | B |
| Comparative Example | CRe-1 | 3.01 | F |
| 1-1 | | | |
| Comparative Example | CRe-2 | 3.45 | F |
| 1-2 | | | |
| Example 1-48 | Re-48 | 1.93 | A |
| Example 1-49 | Re-49 | 1.86 | A |
| Example 1-50 | Re-50 | 2.12 | B |
| Example 1-51 | Re-51 | 2.14 | B |
| Example 1-52 | Re-52 | 2.01 | B |
| Example 1-53 | Re-53 | 2.33 | C |
| Example 1-54 | Re-54 | 1.92 | A |
| Example 1-55 | Re-55 | 1.82 | A |
| Example 1-56 | Re-56 | 1.89 | A |
| Example 1-57 | Re-57 | 1.95 | A |
| Example 1-58 | Re-58 | 1.92 | A |
| Example 1-59 | Re-59 | 1.94 | A |
| Example 1-60 | Re-60 | 2.15 | B |
| Example 1-61 | Re-61 | 1.85 | A |
| Example 1-62 | Re-62 | 2.02 | B |
| Example 1-63 | Re-63 | 2.15 | B |
| Example 1-64 | Re-64 | 1.82 | A |
| Example 1-65 | Re-65 | 2.10 | B |
| Example 1-66 | Re-66 | 2.15 | B |
| Example 1-67 | Re-67 | 1.85 | A |
| Example 1-68 | Re-68 | 1.95 | A |
| Example 1-69 | Re-69 | 2.18 | B |
| Example 1-70 | Re-70 | 1.99 | A |
| Example 1-71 | Re-71 | 2.10 | B |
| Example 1-72 | Re-72 | 1.92 | A |
| Example 1-73 | Re-73 | 2.38 | C |
| Example 1-74 | Re-74 | 2.25 | C |
| Example 1-75 | Re-75 | 1.96 | A |
| Example 1-76 | Re-76 | 1.96 | A |
| Example 1-77 | Re-77 | 2.36 | C |
| Example 1-78 | Re-78 | 1.88 | A |
| Example 1-79 | Re-79 | 2.11 | B |
| Example 1-80 | Re-80 | 1.85 | A |
| Example 1-81 | Re-81 | 1.88 | A |
| Example 1-82 | Re-82 | 2.19 | B |
| Example 1-83 | Re-83 | 2.26 | C |
| Example 1-84 | Re-84 | 1.98 | A |
| Example 1-85 | Re-85 | 2.10 | B |
| Example 1-86 | Re-86 | 2.01 | B |
| Example 1-87 | Re-87 | 1.85 | A |
| Example 1-88 | Re-88 | 1.89 | A |
| Example 1-89 | Re-89 | 1.87 | A |
| Example 1-90 | Re-90 | 1.88 | A |

As shown in Table 4 above, it was demonstrated that the resist compositions of the present invention were excellent in the LWR performance and the defect suppression performance when a pattern was formed by organic solvent development. On the other hand, these performances were insufficient in the resist compositions of Comparative Examples.

### [EUV Exposure, Alkaline Aqueous Solution Development]

An underlayer film-forming composition AL412 (manufactured by Brewer Science, Inc.) was applied to a silicon wafer having a diameter of 12 inches and baked at 205° C. for 60 seconds to form an underlying film having a film thickness of 20 nm. A resist composition shown in Table 5 was applied to the underlying film and baked at 100° C. for 60 seconds to form a resist film having a film thickness of 30 nm.

The resulting silicon wafer having the resist film was subjected to pattern irradiation using an EUV exposure apparatus (Micro Exposure Tool, manufactured by Exitech Ltd., NA 0.3, Quadrupole, outer sigma 0.68, inner sigma 0.36) such that the average line width of the pattern was 14 nm. As a reticle, a mask having a line size of 14 nm and a line:space ratio of 1:1 was used.

The resist film after exposure was baked at 90° C. for 60 seconds, then developed with an aqueous tetramethylammonium hydroxide solution (2.38 mass %) for 30 seconds, and then rinsed with pure water for 30 seconds. Subsequently, this was spin-dried to obtain a positive-type pattern.

The obtained positive-type pattern was used to evaluate the LWR performance and defect suppression performance in the same manner as described above.

The evaluation results are shown in the following tables.

TABLE 5

| | | LWR | |
|---|---|---|---|
| | Resist | performance | Defect suppression |
| | composition | (nm) | performance |
| Example 2-1 | Re-1 | 2.03 | B |
| Example 2-2 | Re-2 | 2.02 | B |
| Example 2-3 | Re-3 | 2.22 | C |
| Example 2-4 | Re-4 | 2.24 | C |
| Example 2-5 | Re-5 | 1.84 | A |
| Example 2-6 | Re-6 | 1.81 | C |
| Example 2-7 | Re-7 | 1.85 | A |
| Example 2-8 | Re-8 | 1.90 | A |
| Example 2-9 | Re-9 | 1.82 | A |
| Example 2-10 | Re-10 | 2.32 | C |
| Example 2-11 | Re-11 | 2.21 | C |
| Example 2-12 | Re-12 | 2.04 | B |
| Example 2-13 | Re-13 | 2.12 | B |
| Example 2-14 | Re-14 | 2.27 | C |
| Example 2-15 | Re-15 | 1.82 | A |
| Example 2-16 | Re-16 | 2.01 | B |
| Example 2-17 | Re-17 | 2.05 | B |
| Example 2-18 | Re-18 | 2.25 | C |
| Example 2-19 | Re-19 | 1.95 | A |
| Example 2-20 | Re-20 | 2.16 | B |
| Example 2-21 | Re-21 | 1.82 | A |
| Example 2-22 | Re-22 | 2.18 | B |
| Example 2-23 | Re-23 | 2.11 | B |
| Example 2-24 | Re-24 | 1.87 | A |
| Example 2-25 | Re-25 | 1.85 | A |
| Example 2-26 | Re-26 | 2.37 | C |
| Example 2-27 | Re-27 | 2.14 | B |
| Example 2-28 | Re-28 | 2.42 | D |
| Example 2-29 | Re-29 | 1.89 | A |
| Example 2-30 | Re-30 | 2.09 | B |
| Example 2-31 | Re-31 | 2.26 | C |
| Example 2-32 | Re-32 | 2.62 | E |
| Example 2-33 | Re-33 | 2.45 | D |
| Example 2-34 | Re-34 | 2.15 | B |
| Example 2-35 | Re-35 | 1.87 | A |
| Example 2-36 | Re-36 | 2.03 | B |
| Example 2-37 | Re-37 | 2.36 | C |
| Example 2-38 | Re-38 | 2.30 | C |
| Example 2-39 | Re-39 | 2.12 | B |
| Example 2-40 | Re-40 | 1.93 | A |
| Example 2-41 | Re-41 | 2.19 | B |
| Example 2-42 | Re-42 | 2.29 | C |
| Example 2-43 | Re-43 | 2.14 | B |
| Example 2-44 | Re-44 | 2.31 | C |
| Example 2-45 | Re-45 | 2.15 | B |
| Example 2-46 | Re-46 | 2.15 | B |
| Example 2-47 | Re-47 | 2.14 | B |
| Comparative Example | CRe-1 | 3.02 | F |
| 2-1 | | | |
| Comparative Example | CRe-2 | 3.44 | F |
| 2-2 | | | |
| Example 2-48 | Re-48 | 1.93 | A |
| Example 2-49 | Re-49 | 1.87 | A |
| Example 2-50 | Re-50 | 2.12 | B |
| Example 2-51 | Re-51 | 2.16 | B |
| Example 2-52 | Re-52 | 2.03 | B |
| Example 2-53 | Re-53 | 2.21 | C |
| Example 2-54 | Re-54 | 1.82 | A |
| Example 2-55 | Re-55 | 1.82 | A |
| Example 2-56 | Re-56 | 1.84 | A |
| Example 2-57 | Re-57 | 1.94 | A |
| Example 2-58 | Re-58 | 1.90 | A |
| Example 2-59 | Re-59 | 1.94 | A |
| Example 2-60 | Re-60 | 2.04 | B |
| Example 2-61 | Re-61 | 1.95 | A |
| Example 2-62 | Re-62 | 2.15 | B |
| Example 2-63 | Re-63 | 2.18 | B |
| Example 2-64 | Re-64 | 1.88 | A |
| Example 2-65 | Re-65 | 2.05 | B |
| Example 2-66 | Re-66 | 2.04 | B |
| Example 2-67 | Re-67 | 1.92 | A |
| Example 2-68 | Re-68 | 1.98 | A |
| Example 2-69 | Re-69 | 2.19 | B |
| Example 2-70 | Re-70 | 1.92 | A |
| Example 2-71 | Re-71 | 2.08 | B |
| Example 2-72 | Re-72 | 1.90 | A |
| Example 2-73 | Re-73 | 2.35 | C |
| Example 2-74 | Re-74 | 2.34 | C |
| Example 2-75 | Re-75 | 1.85 | A |
| Example 2-76 | Re-76 | 1.89 | A |
| Example 2-77 | Re-77 | 2.38 | C |
| Example 2-78 | Re-78 | 1.90 | A |
| Example 2-79 | Re-79 | 2.18 | B |
| Example 2-80 | Re-80 | 1.92 | A |
| Example 2-81 | Re-81 | 1.92 | A |
| Example 2-82 | Re-82 | 2.10 | B |
| Example 2-83 | Re-83 | 2.28 | C |
| Example 2-84 | Re-84 | 1.99 | A |
| Example 2-85 | Re-85 | 2.16 | B |
| Example 2-86 | Re-86 | 2.01 | B |
| Example 2-87 | Re-87 | 1.92 | A |
| Example 2-88 | Re-88 | 1.91 | A |
| Example 2-89 | Re-89 | 1.87 | A |
| Example 2-90 | Re-90 | 1.90 | A |

As shown in Table 5 above, it was demonstrated that the resist compositions of the present invention were excellent in the LWR performance and the defect suppression performance also when a pattern was formed by alkali development. On the other hand, these performances were insufficient in the resist compositions of Comparative Examples.

### [Pattern Formation by EB Exposure and Evaluation: Examples 3-1 to 3-90]

Also in the case where a resist film was formed using each resist composition of Examples and exposed to an electron beam to form a pattern, results with the same tendency as in the case where a pattern was formed by EUV exposure were obtained.

According to the present invention, it is possible to provide an actinic ray-sensitive or radiation-sensitive resin composition that can suppress the occurrence of defects and exhibits excellent roughness performance in the formation of an ultrafine pattern (for example, a line-and-space pattern having a line width of 25 nm or less or a hole pattern having a hole diameter of 25 nm or less), an actinic ray-sensitive or radiation-sensitive resin film formed using the actinic ray-sensitive or radiation-sensitive resin composition, and a pattern forming method and a method for producing an electronic device that use the actinic ray-sensitive or radiation-sensitive resin composition.

While the present invention has been described in detail and with reference to specific embodiments, it will be apparent to those skilled in the art that various changes and modifications can be made without departing from the spirit and scope of the present invention.

What is claimed is:

1. An actinic ray-sensitive or radiation-sensitive resin composition comprising:
an acid-decomposable resin (A); and
the following compound (B) which generates an acid upon irradiation with actinic rays or radiation,
Compound (B):
a compound which has the following structural moiety X and the following structural moiety Y and which generates an acid including the following first acidic moiety derived from the structural moiety X and the following second acidic moiety derived from the structural moiety Y upon irradiation with actinic rays or radiation,
Structural moiety X: a structural moiety which is constituted by an anionic moiety A₁⁻ and a cationic moiety M₁⁺ and which forms the first acidic moiety represented by HA₁ upon irradiation with actinic rays or radiation,
Structural moiety Y: a structural moiety which is constituted by an anionic moiety A₂⁻ and a cationic moiety M₂⁺ and which forms the second acidic moiety represented by HA₂ upon irradiation with actinic rays or radiation,
provided that the compound (B) satisfies conditions I and II below:
Condition I: a compound PI obtained by replacing the cationic moiety M₁⁺ in the structural moiety X and the cationic moiety M₂⁺ in the structural moiety Y in the compound (B) with H⁺ has an acid dissociation constant a1 (pKa) derived from an acidic moiety represented by HA₁ obtained by replacing the cationic moiety M₁⁺ in the structural moiety X with H⁺ and an acid dissociation constant a2 (pKa) derived from an acidic moiety represented by HA₂ obtained by replacing the cationic moiety M₂⁺ in the structural moiety Y with H⁺, and the acid dissociation constant a2 is larger than the acid dissociation constant a1,
Condition II: in the compound (B), the anionic moiety A₂⁻ in the structural moiety Y is an anionic moiety including a partial structure represented by a formula (1) below, 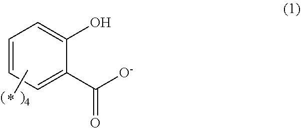
wherein in the formula (1), * represents a bonding site to another atom.

2. The actinic ray-sensitive or radiation-sensitive resin composition according to claim 1, wherein the acid dissociation constant a1 (pKa) is −1.00 or less.

3. The actinic ray-sensitive or radiation-sensitive resin composition according to claim 1, wherein in the compound (B), the anionic moiety A₁⁻ in the structural moiety X is an anionic moiety including a partial structure represented by any one of formulae (2) to (5) below: 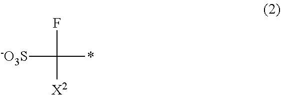
in the formula (2), X² represents a hydrogen atom, a halogen atom, or an organic group, and * represents a bonding site to another atom, 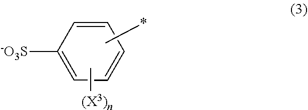
in the formula (3), X³ represents an electron-withdrawing group, n represents an integer of 1 to 4, and, when n is an integer of 2 or more, a plurality of X³'s may be the same or different, and * represents a bonding site to another atom, 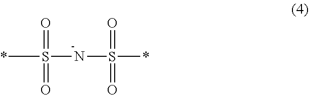 [SMILES: *S(=O)(=O)NS(*)(=O)=O]
in the formula (4), * represents a bonding site to another atom, and 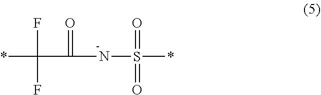 [SMILES: *C(F)(F)C(=O)NS(*)(=O)=O]
in the formula (5), * represents a bonding site to another atom.

4. The actinic ray-sensitive or radiation-sensitive resin composition according to claim 1, wherein the compound (B) has a halogen atom in a cationic moiety.

5. The actinic ray-sensitive or radiation-sensitive resin composition according to claim 1, wherein the acid-decomposable resin (A) includes a repeating unit having a phenolic hydroxy group.

6. An actinic ray-sensitive or radiation-sensitive film formed using the actinic ray-sensitive or radiation-sensitive resin composition according to claim 1.

7. A pattern forming method comprising:
forming an actinic ray-sensitive or radiation-sensitive film on a substrate using the actinic ray-sensitive or radiation-sensitive resin composition according to claim 1;
exposing the actinic ray-sensitive or radiation-sensitive film; and
developing the exposed actinic ray-sensitive or radiation-sensitive film using a developer to form a pattern.

8. A method for producing an electronic device, the method comprising the pattern forming method according to claim 7.
